(19) United States
(12) Patent Application Publication
(10) Pub. No.: US 2024/0000837 A1
(43) Pub. Date: Jan. 4, 2024

(54) INHIBITION OF KIR2DL2 FOR THE ENHANCEMENT OF ADOPTIVE IMMUNOTHERAPIES

(71) Applicant: H. LEE MOFFITT CANCER CENTER AND RESEARCH INSTITUTE INC., Tampa, FL (US)

(72) Inventors: Daniel Abate-Daga, Tampa, FL (US); Miguel Gomez Fontela, Tampa, FL (US)

(21) Appl. No.: 18/253,793

(22) PCT Filed: Dec. 20, 2021
(86) PCT No.: PCT/US2021/073023

§ 371 Date: May 22, 2023

Related U.S. Application Data
(60) Provisional application No. 63/129,856, filed on Dec. 23, 2020.

(51) Int. Cl. A61K 35/17; C12N 15/113; C12N 5/0783; A61K 39/00; C07K 16/28; C07K 14/47; A61P 35/00

(52) U.S. Cl. CPC A61K 35/17; C12N 15/1138; C12N 5/0636; A61K 39/4611; A61K 39/4613; A61K 39/4631; C07K 16/2818; C07K 16/2827; C07K 14/4703; A61P 35/00

(57) ABSTRACT

Disclosed herein is a method for enhancing adoptively transferred autologous or allogeneic immune effector cells (T cells) in patients who are HLA-C1+. In some embodiments, the method involves ablating KIR2DL2 expression in the T cells prior to adoptive transfer. In some embodiments, the T cells are further engineered to express a CAR. Therefore, disclosed herein are enhanced CAR-T cells that are engineered to have ablated KIR2DL2 expression or activity.

### CROSS-REFERENCE TO RELATED APPLICATIONS

This application claims benefit of U.S. Provisional Application No. 63/129,856, filed Dec. 23, 2020, which is hereby incorporated herein by reference in its entirety.

### SEQUENCE LISTING

This application contains a sequence listing filed in electronic form as an ASCII.txt file entitled “320803_2590_Sequence_Listing_ST25” created on Dec. 15, 2021 and having 103,007 bytes. The content of the sequence listing is incorporated herein in its entirety.

### BACKGROUND

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

### SUMMARY

KIR2DL2, for example, contains 2 extracellular Ig domains, and a long intracellular tail. KIR2DL2 binds HLA molecules of the C1 group (Cw1, Cw3, Cw7 and Cw8) resulting in inhibition of Natural Killer (NK) and T cells, through a mechanism that involves the phosphatase SHP1.

Disclosed herein is a method for enhancing adoptively transferred autologous or allogeneic immune effector cells (T cells) in patients who are HLA-C1+. Therefore, in some embodiments, a sample from the subject is assayed to determine their HLA profile and treated with the disclosed enhanced T cells if determined to be HLA-C1+. In some embodiments, the method involves ablating KIR2DL2 expression in the T cells prior to adoptive transfer. In some embodiments, the T cells are further engineered to express a CAR. Therefore, disclosed herein are enhanced CAR-T cells that are engineered to have ablated KIR2DL2 expression or activity.

Also disclosed are isolated nucleic acid sequences encoding the disclosed polypeptides, vectors comprising these isolated nucleic acids, and cells containing these vectors. The lymphocytes disclosed herein can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the lymphocytes are TILs.

Also disclosed is a method for treating a subject with adoptive cell therapy, in a subject, e.g. providing an anti-cancer immunity, that involves administering to the subject an effective amount of an enhanced T cell disclosed herein. Therefore, disclosed are methods of treating cancer in a subject that involves collecting lymphocytes from the subject, treating the lymphocytes ex vivo to inhibit KIR2DL2 expression, and transferring the modified lymphocytes back to the subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates how binding of HLA-C molecules (expressed by target cells) through KIR2DL2 (expressed by T cells) can cause inhibition of CAR-mediated tumor lysis, or TCR-mediated lysis (especially for HLA-C-restricted TCRs).

FIG. 2 shows Nanostring analysis of gene expression in peripheral blood T cells, collected from melanoma and synovial cell sarcoma patients. Pre-infusion samples (Pre) are unmodified T cells; Infusion samples (Inf) are genetically modified T cells as they were administered to patients; Post-infusion (Post) cell are peripheral blood T lymphocytes collected 1 month post adoptive transfer (Abate-Daga et al., Blood 2013).

FIG. 3 shows human peripheral blood T cells were genetically modified to express a second-generation CAR (PSCA2), a third-generation CAR (PSCA3), or GFP as a negative control. Following 14 days of expansion ex vivo, the T cells were administered to immunodeficient mice bearing subcutaneous xenografts of HPAC pancreatic cancer cells. Thirty days after infusion, splenocytes were collected. The gene expression of these post-infusion T cells (Post) was compared to that of the infusion product (Pre). The box plot shows the relative expression values for each condition (in triplicates).

FIG. 4 shows Panc02.03 pancreatic cancer cells incubated with a fusion protein encompassing the extracellular domain of KIR2DL2 linked to the Fc portion of an antibody (KIR2DL2-Fc). Then, cells were incubated with a secondary staining with a PE-conjugated anti-Fc reagent (Anti-Fc), showing binding in over 60% of Panc02.03 cells. Negative controls of unstained Panc02.03 cells, or cells with only secondary staining, were used to establish the gating strategy.

FIGS. 5A and 5B show schematic representation of the KIR2DL2 gene and its protein sequence. FIG. 5A shows KIR2DL2 coding sequence (hg38-NM_014219.2), including exons and both the 5′ and 3′ UTR, is depicted. Guide RNAs (gRNAs) and CRISPR RNAs (crRNAs) are represented by arrows, respectively. FIG. 5B shows KIR2DL2 protein sequence consensus (NP_055034) with the different protein regions. Maps and features were created and represented with SnapGene software (Insightful Science).

FIG. 6A-6H. Alignment of all designed gRNAs and crRNAs within the KIR2DL2 coding sequence. In green, gRNAs (Cas9) are indicated and aligned; in orange, crRNAs (Cas12) are indicated and aligned.

FIGS. 7A and 7B show KIR2DL2 knockout experimental design. FIG. 7A, left table shows the gRNAs or crRNAs, targeting different KIR2DL2 exons, used to disrupt KIR2DL2 expression in 2×10⁵ human T cells known to express the gen. The housekeeping gene HPRT1 was used as a positive editing control. Table on the right shows the expected size, in base pairs (pb), of the unedited or edited PCR products after T7E1 cleavage. FIG. 7B shows the editing efficiency. Forty-eight to seventy-two hours after transfection, DNA was extracted, and every targeted exon amplified to assess Cas9/Cas12 cut by a T7E1 cleavage assay.

FIGS. 8A and 8B show T7 Endonuclease I cleavage assay confirmed KIR2DL2 exon 8 cleavage by the Cas12 nuclease. 2×10⁵ human T cells and Jurkat T cells, known to express KIR2DL2, were nucleofected with a crRNA targeting the HPRT1 gene or the KIR2DL2 exon 8. After 48-72 hours, DNA was extracted and cutting efficiency was assessed with a T7E1 cleavage assay. FIG. 8A shows the expected size of KIR2DL2 exon 8 amplicon is 899 bp, whereas the edited cells shows two bands of 729 and 170 bp (white asterisks and arrows). FIG. 8B shows cleavage efficiency was calculated as the percentage of DNA cleaved by using the following formula: (Fragment1+Fragment2/Total intensity)*100. Total intensity was calculated by the sum of intensities of the fragment 1, fragment 2 and fragment parent. Results shows an average efficiency of 45% in Jurkat T cells and 18% in human primary T cells. HPRT1 cleavage was measured as a nucleofection control. Bar represent the mean±SD of two independent experiments.

FIGS. 9A and 9B show KIR2DL2 bicistronic expression model. The MSGV1 retroviral vector containing the PSCA-CAR 28t28z followed by the KIR2DL2 CDS separated by a P2A peptide was designed and used for viral production. OKT3-stimulated PBMCs were transduced with viral particles containing both the PSCA-CAR 28t28z or the CAR+KIR2DL2, and the surface expression of both proteins were analyzed by flow cytometry 7 days after transduction. FIG. 9A shows the schematic representation of the retroviral vector. FIG. 9B shows a representative PSCA-CAR and KIR2DL2 expression 7 days after transduction. The P2A peptide allows the expression of both molecules at the same time in different transcripts with high efficiency. Untransduced cells (UTD) were used as a negative control.

FIGS. 10A and 10B show HLA-1 deficiency impairs KIR2DL2 binding to Panc0203 tumor cells. KIR2DL2 expressed in the T cell membrane interacts with HLA-C1 expressed in the tumor cell membrane. A PSCA-expressing cell line (Panc0203) was engineered through CRISPR-Cas9 with a gRNA targeting the β2-microglobulin gene to abrogate HLA-1 expression (Panc0203 ρ2-m⁻). Edited single cell clones were isolated and purified by FACS. HLA-1 expression and KIR2DL2 chimera (KKIR2DL2-Fc) binding were assessed by flow cytometry. FIG. 10A shows representative dot plot analysis showing HLA-I expression and KIR2DL2 chimera binding to unedited Panc0203 cells or Panc0203 P2-m-. FIG. 10B shows graphical representation of the percentage of cells expressing HLA-I and binding the KIR2DL2 chimera and the mean florescence intensity (MFI) of that expression and binding. Data is presented as a mean±SD of two independent experiments. Results shows that lack of HLA-1 expression in tumor cells impairs KIR2DL2 binding. These cells will allow us to create an interaction model for the assessment of KIR2DL2 biology both in vitro and in vivo by comparing their ability to be killed by PSCA-CAR T cells expressing, or not, KIR2DL2.

FIG. 11 shows human T cells transduced to express the PSCA CAR (28t28z) or the PSCA CAR together with the KIR2DL2 protein were incubated with Panc0203 cells unedited or CRISPR/Cas-edited to abrogate their β2m expression, at different effector:target ratios.

FIGS. 12A to 12D show KIR2DL2 impairs CAR T cell cytotoxicity in vitro against different tumor cells. Human T cells transduced to express the PSCA-CAR 28t28z or the PSCA-CAR together with the KIR2DL2 protein were incubated with Panc0203 cells unedited or CRISPR/Cas9-edited to abrogate HLA-1 expression. Cytolysis was assessed by a Real Time Cytotoxicity Assay (RTCA) and percentage of cytolysis (% cytolysis) calculated using the RTCA software Pro (Agilent Technologies, CA, USA). FIGS. 12A and 12B show the % cytolysis at different effector:target ratios (E:T) after 24 hours incubation with PSCA⁺/HLA-I⁻Panc0203 cells (FIG. 12A) or PSCA⁺/HLA-I⁻ Panc0203 cells (FIG. 12B). FIGS. 12C and 12D show the % cytolysis at different effector:target ratios (E:T) after 24 hours incubation with PSCA⁺/HLA-I⁻HPAC cells (FIG. 12C) or PSCA⁺/HLA-I⁻ HPAC cells (FIG. 12D). Data are shown as a mean±SD of triplicates for every E:T ratio. Statistical significance was calculated with a two-way ANOVA test comparing the % cytolysis between the PSCA-CAR and the PSCA-CAR/KIR2DL2 T cells. Ns (non-significative); **** p<0.0001; *** p<0.0005; ** p<0.005; * p<0.05. For both tumor cells lines, KIR2DL2 interaction with its HLA-C ligand seems to significantly impair CAR T cell function, whereas cells that lack HLA-I expression are efficiently killed by both CAR T cells. These results suggest an inhibitory role of KIR2DL2 in CAR T cell biology, thus allowing us to target this marker as a therapeutical approach to enhance CAR T cell adoptive transfer therapy.

FIG. 13 shows KIR2DL2 expression impairs CAR T cell IFN-γ secretion. Human T cells expressing the PSCA-CAR or the PSCA-CAR together with the KIR2DL2 molecule were cocultured for 24 hours with both PSCA⁺/HLA-I⁺ or PSCA⁺/HLA-I⁻ Panc0203 and HPAC tumor cells. Supernatants were collected and IFN-γ was measured by ELISA. Quantification of IFN-γ in wells with only tumor cells and media, or tumor cells and untransduced T cells (UTD) was used as a control. Data represents the mean±SD of three independent measurements. In accordance with the cytotoxic assay, CAR T cells lacking KIR2DL2 seems to produce more IFN-γ when cocultured with cells lacking HLA-I expression, thus suggesting a suppressive effect for the KIR2DL2 molecule.

FIGS. 14A and 14B show KIR2DL2 mRNA expression is upregulated both in patients who received TCR-transgenic T cells and in PSCA-CAR T cells. FIG. 14A shows mRNA quantification of relevant genes for the immune system showing an upregulation in KIR2DL2 post-infusion. Pre-infusion samples (Pre) are unmodified T cells; Infusion samples (Inf) are genetically modified T cells as they were administered to patients; Post-infusion (Post) cell are peripheral blood T lymphocytes collected 1 month post adoptive transfer. Right panel. Human peripheral blood T cells were genetically modified to express a second-generation CAR (PSCA2), a third-generation CAR (PSCA3), or GFP as a negative control. Following 14 days of expansion ex vivo, T cells were administered to immunodeficient mice bearing subcutaneous xenografts of HPAC pancreatic cancer cells. Thirty days after infusion, splenocytes were collected. The gene expression of these post-infusion T cells (Post) was compared to that of the infusion product (Pre). The box plot shows three independent replicates of the relative expression values for each condition. FIG. 14B shows flow cytometry analysis of PSCA2-tranduced cells confirms the upregulation of KIR2DL2 protein post-infusion.

FIGS. 15A and 15B show KIR2DL2 impairs CAR T cell cytotoxicity in vivo. KIR2DL2 role in CAR T cell effector function was assessed in vivo using a NSG mouse model. FIG. 15A is a schematic representation of the in vivo CAR-T treatment protocol. HLA-I expressing (PSCA⁺/HLA-I⁺) or HLA-deficient (PSCA⁺/HLA-I⁻) tumor cells were injected into the flank of NSG mice. Mice were randomized into four groups (n=5 each group) and treated with 5×10⁶ PSCA-CAR or PSCA-CAR/KIR2DL2 T cells; GFP-transduced T cells were included as controls. Tumor size was measured by caliper three times a week. FIG. 15B shows tumor growth curve in each group was shown as mean±SEM. Linear regression analysis was used to calculate the tumor growth slope, and the statistical differences between the slopes were calculated using one-way ANOVA. Non-significant (ns); * p<0.05; ** p<0.005. As shown in vitro, KIR2DL2 expression together with the PSCA-CAR impairs T cell cytotoxic function in the presence of KIR2DL2 ligand, whereas the absence of its ligands allows the cells to properly react against target cells.

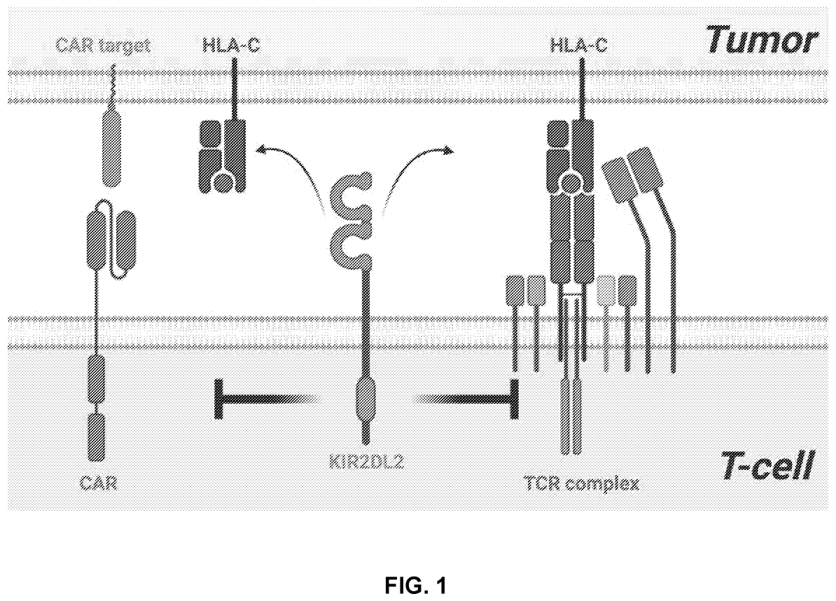

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### KIR2DL2 Ablation or Inhibition

In some embodiments, immune effector cells are modified ex vivo to inhibit or ablate KIR2DL2 expression or activity, and then adoptively transferred back to the subject.

As used herein the terms “inhibit” and “ablate” connote a partial or complete reduction in the expression and/or function of the KIR2DL2 polypeptide encoded by the endogenous gene. Thus, the expression or function of the KIR2DL2 gene product can be completely or partially disrupted or reduced (e.g., by 50%, 75%, 80%, 90%, 95% or more, e.g., 100%) in a selected group of cells (e.g., a tissue or organ) or in the entire animal.

KIR2DL2 is a co-inhibitory receptor that may act as an immune checkpoint mechanism in the modulation of T cell activity. Its inhibitory effect is well characterized in natural killer (NK) cells, but little is known about its mechanism of action in T cells.

Multiple technologies exist to induce disruption of a gene, including CRISPR/Cas-based system, TALEN, MegaTAL, or other sequence-specific nucleases. In addition to genetic ablation using nucleases, other methods can be used to achieve reversible inhibition of KIR2DL2 expression or activity. For instance, CRISPR/Cas systems can be engineered to induce reversible inhibition of gene transcription. Alternatively, small molecules or monoclonal antibodies could be dosed in vivo to block the interaction between KIR2DL2 and its ligand. Small molecule inhibitors that target the phosphatase SHP1 could prevent KIR2DL2 inhibition. Each of these and other approaches have different advantages and disadvantages. For example, genetic ablation has the advantage of inducing a permanent disruption in this pathway only on adoptively transferred T cells, which may allow for long-term prevention of KIR2DL2-induced inhibition without affecting other KIR2DL2-expressing cells in the immune system. On the other hand, transient inhibition with small molecule inhibitors or antibodies may allow for real-time control of the magnitude and duration of the checkpoint blockade.

In some embodiments, the ex vivo KIR2DL2 ablation involves contacting the immune effector cells with a targeted nuclease, a guide RNA (gRNA), an siRNA, an antisense RNA, microRNA (miRNA), or short hairpin RNA (shRNA).

In some embodiments, the targeted nuclease may introduce a double-stranded break in a target region in the KIR2DL2 gene of the immune effector cells. The targeted nuclease may be an RNA-guided nuclease. In some embodiments, the RNA-guided nuclease is a Cpf1 (Cas12) nuclease or a Cas9 nuclease and the method further comprises introducing into the immune effector cell a gRNA that specifically hybridizes to the target region in the KIR2DL2 gene. In some embodiments, the Cas12 nuclease or the Cas9 nuclease and the gRNA are introduced into the cell as a ribonucleoprotein (RNP) complex. Therefore, in some embodiments, the ex vivo KIR2DL2 ablation involves performing clustered regularly interspaced short palindromic repeats (CRISPR)/Cas genome editing.

In some embodiments, KIR2DL2 has the amino acid sequence:

mslmvvsmacvgffllqgawpHEGVHRKPSLLAHPGRLVKSEETVILQCWSDVRFEHFLLH REGKFKDTLHLIGEHHDGVSKANFSIGPMMQDLAGTYRCYGSVTHSPYQLSAPSDP LDIVITGLYEKPSLSAQPGPTVLAGESVTLSCSSRSSYDMYHLSREGEAHECRFSAG PKVNGTFQADFPLGPATHGGTYRCFGSFRDSPYEWSNSSDPLLVSVTGNPSNSW PSPTEPSSKTGNPRHLHiligtsvviilfillfflHRWCSNKKNAAVMDQESAGNRTANSED SDEQDPQEVTYTQLNHCVFTQRKITRPSQRPKTPPTDIIVYTELPNAESRSKVVSCP (SEQ ID NO:1, NP_055034), comprising a signal peptide (lower case and italicized), EXTRACELLULAR DOMAIN (capitalized), transmembrane domain (lower case and bold, and INTRACELLULAR DOMAIN (capitalized and bold).

In some embodiments, KIR2DL2 gene has the nucleic acid sequence:

| (SEQ ID NO: 2) | |
|---|---|
| 1 | cgcggccgcc tgtctgcaca gacagcacca tgtcgctcat ggtcgtcagc atggcgtgtg |
| 61 | ttggtgagtc ctggaaggga atcgagggag ggagtgcggg gatggagatc ggggcccaga |
| 121 | gttggagata taggcctgga agtggagtta tgggcctaga gatggagtga tgggcctaga |
| 181 | agtggagatc tgggcctgga gtggagatat gggcctggag gttgagatat gggcctgcag |
| 241 | tagagatatg ggcttgtagt ggagacatgg gcctggagat ggagatatgg gcctggagat |
| 301 | ggagatatgg gcctgcagta gagatagggg cctggagtgg agatatgggc ctggagtgga |
| 361 | gatatgggcc tgaagtggag atatgggcct ggaggtggag atatgggcct ggaggtggag |
| 421 | atatgggcct ggagtggaga tatgggtctg gaggtggaga tacgggcctg cagtagagat |
| 481 | atgggcctgg agtggagata tgggccagga gtggagttat gggcctagag gtggatatct |
| 541 | gggcctggag tggagatatg ggcctaggaa ggagatatgg gcctgggtgt ggagatatgg |
| 601 | gactggagag gtgatatggg cctggagtgg agatatgggc ttagggtgga gttctgggcc |
| 661 | tggggcggag atatgggact ggattggaga taggggccta gggtggagat ctgagcctgg |
| 721 | attggcgata tgggcctagg gtggaaatat cagcctggag tggagatatg ggcttggggt |
| 781 | ggggatatgg gcctggaaac tgggtctctg cacagccgac agccctgttc ttgggtgcag |
| 841 | gtaggcactg agggtgagtt taacttcagc ccaggaaggg cctggctgcc aagactcaca |
| 901 | gcccagtggg ggcagcaagg gagggctggt tcgcctgcag atggatcgtc catcatgatc |
| 961 | tttctttcca gggttcttct tgctgcaggg ggcctggcca catgagggtg agtccttctc |
| 1021 | caaaccttcg ggtgtcatct ccccacataa gaggattttc ctgaaacagg agggaagtcc |
| 1081 | tgtcggggag tctctcataa actaggaaga gaggaccctg gggtgctcag cccacatttc |
| 1141 | tgacctcgcc tccctggcct ctcaacccct tggcagagtc aagttctgtg gggaccaggg |
| 1201 | ttagactggg gtgctcaaag ctggggtgtg tggttgggaa gtggtaggaa cagcagatcc |
| 1261 | tctgaggaca aaggtgttac tcacacactt cagcgtttcc atgatggtag gggctgcagt |
| 1321 | gtggctgctg tcattctacc agaagaggtg ggaaaccaca gccatggccc tgacattcca |
| 1381 | aatcctctga tgggggctca gttgtttatt ttcgttcagg catccgctga tatccattca |
| 1441 | caaaggacat gccctccacc tcatgtctac cctgtgttgt tttatgtgag taatcttaca |
| 1501 | gtatcaaaat ctagtaggag tctctttact cagcacttgc tcaaagttct cagctgaggc |
| 1561 | ttttgttgta gggagacacc atgtctttgc gggatgggtc cttccttcag ccctgggcac |
| 1621 | caaggtgtga tagtagccat agaaacgtgg aaagcgagga gaatcttctg agcacaggga |
| 1681 | gggaggggca gttccacatc ctcctctcta aggcggcgcc tccttctccc caaggtggtc |
| 1741 | aggacaagcc cttgctgtct gcctggccca gccttgtggt gcctctagga catgtcattc |
| 1801 | ttcggtgtca ctcttatctt gggtttaaca acttcagtct gtacaaggaa ggtggggtgc |
| 1861 | ctgtccctga gctctacaac agaatattct ggaacagcct tttcatgggc cctgtgaccc |
| 1921 | ccgcacaaca gggacataca gatgtcgggg ttcacacaca cactccccca gtgggtggtc |
| 1981 | agcacccagc aaccccctgg tgatcgtggt cataggtcag agggctcctg tottggattc |
| 2041 | tccttgtccc acctcctgaa tcccagagct tctggtgggc atgtccttga gggtcccatc |
| 2101 | acgcaggccc tgactgtatt tgtggtaaag ggggattgaa tacagggaaa tgggtgctgt |
| 2161 | ggtgggaaga ataattgtcc ccagtgatga ctacattcta atccctggag tctgtgacta |
| 2221 | tgtatgttat aggggaaggg actgaagggg aagatggagc tcatggggag acagcctgga |
| 2281 | ctgtcccact gggctcagtg taatcacaag ggtgcacatg aaaggaggag gaagagggga |
| 2341 | gtggggatta gagcagtcca gtggaagtct tcaccagctt tgaaggtgga ggaaggccaa |
| 2401 | gagccatgaa tgcaggtggc ctatagaggc tggaaaagtc aaggaactga ttctccagag |
| 2461 | tctccagagg gaacaaagcc ctgcagatgc cttgatttta gcccaggaaa aatagggtcc |
| 2521 | aatttctgtc tccagtactg gaaggtgtca gtgtggtctc tcctgcttcc atgcttctga |
| 2581 | taattttgta cagcagcaac aggaaaccaa cactggaacc caggtcaagg acaagttaag |
| 2641 | aaacaaccca aggaaagcca ggcatggtgg caggcgcatg taatcctagc gactcaggag |
| 2701 | gctgagggca ggagaatcac ttgaacccag gaaacagagg ttgcagtgag cctagaccac |
| 2761 | accacttcac tccagcctgg gtgaaggagt gagactctgt ctccaaaatt aattaattaa |
| 2821 | ttaaagaaac caaagaagga gaaggttggc taccctgaga tcagcaaggg tgggatgatg |
| 2881 | atgccaccac caggctccat ccacataggg aggggttgat actcctccaa ccagcaccag |
| 2941 | gagccagcct atggaagctg gcaccatgga gaaggcacag gcatggcaag agtggctccc |
| 3001 | agtccccacc aggaacaggg tgtgtggaca ctggtgcctg ccttattcat cagttcatat |
| 3061 | cttctgccaa ggattgcaat tcatccaaaa gagattgaac caggctgata agagcctgga |
| 3121 | tgtgcagcct atcctggttc ctctttcacc cccacataaa cagcaggaaa gacattagtg |
| 3181 | tgaaatagat acaacacccc aagagatgag gctaagccca gtgggaaggg aatcagaggc |
| 3241 | tactagagac agagggacag agaagaggga gggagacaga tggaaggacc tgcaccagga |
| 3301 | gttaagggca cagaaaagaa catgaagaca cagagaggaa ggagagagac agacaccagc |
| 3361 | aaggggaagc ctcactcatt ctaggtgcca tggatgggat gataaagaga gacaccttct |
| 3421 | aaactcacaa cctctcttcc taggagtcca cagaaaacct tccctcctgg cccacccagg |
| 3481 | tcgcctggtg aaatcagaag agacagtcat cctgcaatgt tggtcagatg tcaggtttga |
| 3541 | gcacttcctt ctgcacagag aagggaagtt taaggacact ttgcacctca ttggagagca |
| 3601 | ccatgatggg gtctccaaag ccaacttctc catcggtccc atgatgcaag accttgcagg |
| 3661 | gacctacaga tgctacggtt ctgttactca ctccccctat cagttgtcag ctcccagtga |
| 3721 | ccctctggac atcgtcatca caggtgagag tgtccggaca ttctcattgt cattgggctg |
| 3781 | cagagtgaat gatccacgac ttggaacccc caggtagttg taaggaagat gagcttggta |
| 3841 | ttcttatgga gagagactga cttgctgagg tttgtaccaa cagagacaga gaaacaggag |
| 3901 | acacaagtac agaccaggtg tcataacgga ggacagacac aggggccata cagggagtta |
| 3961 | gaaaagacag aaagagttaa aagagacaga cagacagaca tgtcccagag agaggtgtcc |
| 4021 | ctccatgctg actttgctca cagacctggc acaggttaga agtttcattt ctgttttacc |
| 4081 | tccacaaagt gttctctacc aggagaaccc aaggacaccc atatttctga cctgagttgg |
| 4141 | gccctgtggc ctcaggcctt gtggcaccta caggccatgt ttattctgac acctctgcct |
| 4201 | tccatgtaat ggagagtaac cgtcccagga tatcatggcc ccagaacacc aacccctgta |
| 4261 | tgctgtgtga acttgtggtc tccagactgg attctgaggc tcacattcca aataacccca |
| 4321 | catatgaaag gatcactgag aggcacagag aaaaatcagg aacaccaaaa agcaaagaca |
| 4381 | taaacacacg gagaatgagc cagaggaagg agattgagag actcacagac acataaagag |
| 4441 | agagaaaaga gggcagagga gtggtgagaa tgatggcagg gagcagagaa aagcactaaa |
| 4501 | attagagtcc tgagagagag gcacaaggac atagaaacat ggagatgtgg ggatgaattg |
| 4561 | cagagattcc aaagagagct agagagaccg agaggcagag caatacagat gatagatgga |
| 4621 | tagatataga tagatgataa ataggtagat gatagataat aggttaaaga tacatagatg |
| 4681 | atgattgatt gattcattaa tagataatac atagagatga tgatgatgaa gacagataat |
| 4741 | acgtacagat agagaggcag acagaaatca tagagagaga gatgatacat acatataaat |
| 4801 | aacagatgat tgatggatag atagacaact gatagataca tagatgatat atagatatag |
| 4861 | atgacaggta gagaatttgt agataggcac cgaatagata aatagataga togacagata |
| 4921 | atagatagaa atatgcagaa agttatgaac aggacacaac gtgagaaact tagaatttaa |
| 4981 | aaaagtaaca tcaagtcaac caacccaagg agagtcagag agaataaaac aatccaaaaa |
| 5041 | cggaaaacat atctagaggt ggggaagcga ggtcagagac ctagagagac agagaaggtg |
| 5101 | gaagaaggaa atagatatga agagagatgg ggtggagggt gagagagaga gagagagagc |
| 5161 | attaggtcat agagcagggg agtgagttct cagctcaggt gaagggagct gtgacaagga |
| 5221 | agatcctccc tgaggaaaat gcctcttctc cttccaggtc tatatgagaa accttctctc |
| 5281 | tcagcccagc cgggccccac ggttctggca ggagagagcg tgaccttgtc ctgcagctcc |
| 5341 | cggagctcct atgacatgta ccatctatcc agggaggggg aggcccatga atgtaggttc |
| 5401 | tctgcagggc ccaaggtcaa cggaacattc caggccgact ttcctctggg ccctgccacc |
| 5461 | cacggaggaa cctacagatg cttcggctct ttccgtgact ctccatacga gtggtcaaac |
| 5521 | tcgagtgacc cactgcttgt ttctgtcata ggtgaggaaa ccccatatct gtctcatgtc |
| 5581 | ctatgatcct agagccttag ctgaggagct tcctgctgat gatggagata agcatggaca |
| 5641 | gatgcagaga gaagacgaag cttgggtgtg agggagggat cagggcacag gatggcagac |
| 5701 | agggcacctc caaaccctcc tacacggcct gcatgaaggc ccgcggccag ggctccaggc |
| 5761 | acacaggcag atggagaaag cggtcaggag agacccagag gagggagact gggctcagtt |
| 5821 | tgggaagatc agaggttccc tcagcccctc aacattaccc atttcccaga agcccatcct |
| 5881 | ggcctctcac ccacacaggg atgtcatcac cagcaacccc tacacccttt acttttgttt |
| 5941 | gaagaaatat ttattgagga taaatatacc tatatagctt accaccttta acattttttt |
| 6001 | tttttttgag gcagagtcta gctctgtccc ctatgctgca gtgcagtggc acaatctcag |
| 6061 | ctcactgcaa cttccgcctc ctgggttcaa gtgattctcc tgcctcagcc acctgagtag |
| 6121 | ctggtgctac aggcgcgcac caccacgcca ggctactttt tgtattttta gtagagaggt |
| 6181 | ggtttcacca tgttggtcga gctggtctcc aactcctgac cacgtgatcc acccgcatct |
| 6241 | gcctcccaaa gtgctgggat tacaggcatg agccaccact cccagccaca tttaccattt |
| 6301 | ttaagtgtaa agtctagtgg tcataaatac atttataaat atatatatat atatatgtat |
| 6361 | gtatatatat atatatatat atatatatat atatattttt tttttttttt ttaccctcca |
| 6421 | cccttttctt cctggcctct ggaagccacc attctactct ctaccttcat gagatccacc |
| 6481 | ttttagctct gtatatgggt gagaaatggg aatctttgta atgacttgca gttccatcca |
| 6541 | tgtggctgca aatatcagga tgttattctt tctatggatg agtagtctcc actgtgcgta |
| 6601 | tgtactacat tctctctatc cattcatcca ctgatgggca ggtaggttga ctccacatct |
| 6661 | tggctactgt gaacagtgct gcaccaatca tacgagtgca gatatcactt cgatatattg |
| 6721 | atttactttc ctttggatat aaacccagta gtgaaattgc tggatactat gaaagttctc |
| 6781 | tttttagtta ttcgtttgtt gttttgtttt tgtttttgag acagtttccc tctgtgccca |
| 6841 | ggctggagta caagtgatgt catcttggct cattgcaacc tctgcctcct gggttcaaat |
| 6901 | gattttccta cctcagcctc cctagtagct gggattacag gtgcacgcca ccatgcctgg |
| 6961 | ctactttttg gtttttttag tatagatggg gtttccccat gttggctggg ctgctctcaa |
| 7021 | actcatgacc tcaactgagg tgtccgcctc ggtctcccaa agtgccggga ttacaggcat |
| 7081 | gatccacctc acccaacctc tttttagttc tttaaaggac ttccacactt ttctccgtaa |
| 7141 | aggctgtact aatttacact cctaccaaca gggtattagg gttctccttt ctctaccact |
| 7201 | ttggcaggat ttcctttgcc tgtcttgcag ctaaaagcca ttttacttta tttcatttta |
| 7261 | ttttgagatg gagtttcgct cttgtcaccc aggctggagt gcagtggtgc gatctcggct |
| 7321 | caccacaacc tccacctccc aggttcaagc gattctcctg cctcagcctc ccgagtagct |
| 7381 | ggaattacag gcacacgcca ccacgcccga ctaatttttg tatttttagt agagacagtg |
| 7441 | tttctccatg tgggtcagac tggtctcaaa ctcccgacct tatgagattc acccacctca |
| 7501 | ggctctcaaa gatctaggat gacagacgtg agccaccacg cccggcctaa aagccatttt |
| 7561 | aatggggtga gatgaaaact cactttgatt ttaatttgcg tttctctgat gatgagtgat |
| 7621 | actgagcagt ttttcgtatg tggggaaatt tcatgtcttt tgctcctgtt tcaattaaat |
| 7681 | catttgtttt attgagttgt ttgagcttct tatatttcta gttattaatc ccatctcaga |
| 7741 | tgcatagttt gcacatattt gctcccaatc tgtgggttgt ctcttcactt tgttggttta |
| 7801 | tttttagcgg tgcagaagtt gcttagcttg aggtaatccc aatggtctat ttttgcttcg |
| 7861 | attacttgtg ttttgaaggt ttaaaacaaa atgtcttcct tcagacaaat gtcctggagc |
| 7921 | atttccccaa tattttcttc tacgtgtttc ataggttcag gccttagact cacatcttta |
| 7981 | atccattttc atttgatttt tgtgtatggt gacaggtaga ggtgcagttt cattcctctg |
| 8041 | catgtagatg tccaggtttc cctgcactgt ttattgaaaa gactgtcctt tcctgattgt |
| 8101 | gagttcttgg cacctttgtc aaagtccatt ggatgggctg ggcatggtga ctgacacctg |
| 8161 | caatttcagc actttgggag cccaaggcgg gtggatcacc tgaggccagg agttcaagat |
| 8221 | tagtctggcc gacgtgatga aacatcgtct ccactaaaaa tatataaatt agctgagcat |
| 8281 | ggtggtcagc acctataata ccactactca ggagtttgag gccagagaat tgattgaacc |
| 8341 | caggaggctg tggtggcagt gaaccgagat tgcacctctg cactccagcc tgggtgacag |
| 8401 | agcgagactc catctcaaaa gaaaaaagaa aaaaacattg gatgtaaatg catggattat |
| 8461 | atttgtgttg ttcattctgc tccattgttc tatgtgcctt tcttcatgcc aacatcatgc |
| 8521 | tgtcttgctt actacagctc tgtaacatat tttgagatca ggtagtgtga tgctcctgtt |
| 8581 | ttctctttat accttgaagt ctcaagacaa tgggcgtcac atacaaaaat tatggaaaaa |
| 8641 | aggatcccag gactcccagg gcccaatatt agataacaga gtgttggcca tgaaccaacc |
| 8701 | tcaaagattt ccattgagta gaggacagac accctcattt cctcacctct ctcctgtctc |
| 8761 | atgttctagg aaacccttca aatagttggc cttcacccac tgaaccaagc tctaaaaccg |
| 8821 | gtgagtacag aaccctctta tatccgcttt tggaaacctg gggaggtaga aaccttcgat |
| 8881 | gcaggcattg actcagcatc tcgcagctct gacattgtac gcctgtcttc taccatctcc |
| 8941 | gaactccaga tactccaaca gcgaaaggga tctgggccca acctagggct cagtgaaatc |
| 9001 | tcttaatctc tcattttatg gagctgagac ctcctacaag ctagaagaat gattgccaat |
| 9061 | ctgacatcct tctcaggaaa aatgcaatgt ttgttctgcc tgcattccta actggaggat |
| 9121 | aaattcctgg gggcttgaga gagggaaggg aagggaacat ctgatgaggg cgaggtgttt |
| 9181 | tagagaagtt ccacttgcca aggaatgaat tactgttggt catgaagcaa ccctggctga |
| 9241 | ctcagcagag caacagcctt gccgtaacag agaacggagc tcatgcacgc acacttcgac |
| 9301 | tcactgactc attcagccac ggccccatgc tcaggctgtg cagtgcggaa ccttttccta |
| 9361 | ttgttgccat aacaaatttc cacaagattc gtgggtgaaa acaaaacggt tttttaatta |
| 9421 | tottacagtg ctgtagctca aagtaggaag tgcatcttac tgggctaaaa tcaaggtgac |
| 9481 | agcaaggctg ccttccctct gaggattcca ggcaagaatc tgcttctcac ttgtcccagc |
| 9541 | ttctaaaggc tcccagttcc ttggctcctg gtccccttcc tccttcctca aaacccacaa |
| 9601 | agactggtca catctcacat ggcatcactc agtgccttct tccttaccac acctctttct |
| 9661 | ctgaatgctg ctctcccttc ttccttatct tttgaaaact tggggattct attgggttca |
| 9721 | ccaagatgaa aatccctcat aatctcctgg aaatcatcca ggataccctt gttttaagtt |
| 9781 | cagctgatta gcaaccgtaa ttccatctac aatcttcatt cctcctttcc atgtaaaata |
| 9841 | acatattcac aaggtatgga ggctaggaca gggacatttt ggggtgggac agcattctcc |
| 9901 | tgccttccac aaacagtgaa caagatgcat ttggcctctg cccttgggac actgatattg |
| 9961 | cagatggtta aatgggaggg cagaaaatga atgcacaagt ggatctataa atgaatgatc |
| 10021 | cattgggaag catctgtgca tgaaatctat tttttgtttg ttcttttgtt tattgagaca |
| 10081 | gagttgccct ctgtcttcca ggctacagtg cagtgtcacg atcttggctc actgcaacct |
| 10141 | gcttctcctg gattcaagtg attctcctgc ctccgcctct cgagtagctg ggattacagg |
| 10201 | caactgccac cgtgcccggc taattctttt tgtatatttt ttgtagagag gatgtttcac |
| 10261 | cacgttggcc aagcttgtct gaaactccca acctcaagtg atccgaccgt ctcagcatgc |
| 10321 | caaagtaatg ggactacagg cgtgagccac tgtgcccagc cagaattcaa aatcaataat |
| 10381 | agataatgct gagtgtatga tttcaggtga caaagaaggt ctcactattc agatatttgt |
| 10441 | gacattaatg aaaaacacgg attgaacccc tgaaagattg gcggaaggat tttgcacaca |
| 10501 | cagctgtcag ccgtgaaggc acaaaggtga aaacaatctg atgtggaagg aagaggctct |
| 10561 | tcctcaaatg ctgggaatga tgtggggaga atgacaagat gactgtggag agacggagag |
| 10621 | cacactgggt acacaggaaa ctaaggagga acaaggagtg tgtgtttgac actcacagcc |
| 10681 | attggattca cctcggggta gccaggaatc cctacatgat taatatgact gacatgaaaa |
| 10741 | taagggaggc tcagttgcat aactggaatc taggagaccg tggaaaaggc aattgccgcc |
| 10801 | ccactggtga aatgtggtgc tgatttagac actaaatgaa tgaagtagat ggatataaga |
| 10861 | taggtttgtg aggtagaatc attgactgga aaggcttgct gggtttgatt ttcctacttg |
| 10921 | tttaatcctc gcttaattaa tttctttctg agatttattc atcctacaca taaatcaata |
| 10981 | cctggcaaag gagtgacaga tatatgaggg gtggtggaaa tgaagagacc tattatagca |
| 11041 | taatatacaa gtctgtgaac ggtggctcac gcctgtaacc cagcactgca ggaggccaag |
| 11101 | gcgggtggat cacatgaagt cagcagttcg agaccagcct ggccaacatg gtgaaaccct |
| 11161 | gtctctagga aaaacacaaa aattagccga gcatggtggt gcatccctgt aatcccagct |
| 11221 | cctactctgg aggatgaagc aggagaatga cttcaaccca ggaggtggag gttgcagtga |
| 11281 | gtggaggttg catcactgca ctccagcctg ggtggcacaa ggagactccg tctcaaaaaa |
| 11341 | taaaaataag aaatgcataa atataaatat aatataacac acgcaaatga caaagggacc |
| 11401 | tgaattccaa tcatgatttt tctatttctc tataattact tctttgatcc tttatcttat |
| 11461 | ccattaggca atgagcctaa aacctcttcc ctatttggct ttctgtgagc atgagatcat |
| 11521 | atagaaaatg tgaaagtccg ctgaatcctc cagcacagat cctggaatag agaaagtgct |
| 11581 | ctggtcatca caaaaaaaac ttgcccactc acccaaatcc cccacctcac ccctacttcc |
| 11641 | aatcacctgt ggagattcag gtagaccatg gggaggtaaa cattaacact ccttggagtg |
| 11701 | agtccagatc ttggaatcag agatcagcga cagcactagc tcctgctccc ctttcctact |
| 11761 | aattcacagg aggacaggtg gtattgaagc aatagatggc cgagggggtg gtccttcccc |
| 11821 | cagcctctcg ggtagaacag cagcctaata tgtgtctccc gagatcacaa agagcagcag |
| 11881 | gtttcacacg ggcttcaaca ctatttcctg gccgtttgac ataagagaat tctatttcgc |
| 11941 | tttttttatc ttgatttcac ttttgttttc tttccttgga gaatgcaagt tgtttgattc |
| 12001 | aagaatgctg tggatgtaga aaccctaaag cacattcgct gtgaatcaat cccagtccag |
| 12061 | tcttcccaga gaagactcta aacacctcct ggactgcacc tgggcctatg ccaattccta |
| 12121 | tcactcaccg tcactccagg gagacagaac acacagagaa tacgttacat aggcaggttc |
| 12181 | attactaaca gataagcagc gagtgacaac agaaacctat atttcaatgt gagccagtcc |
| 12241 | ctcaaggctc agaaaagctc ctcgggacat atggagtcac cccatttgca gtgtagctgc |
| 12301 | gggaagccag aaagcagccc agcctgggtt ttgtaccctg gagccacagg aagcactcag |
| 12361 | ctaaagcact gcatgacgtc ctccaggaag aacaggaaga cagcccaggg tgttctgaga |
| 12421 | cgttcctcct gatctcagga agttgctgtc ttaggccatt tttgttgctc taaaggaaca |
| 12481 | cttgagcctc ggtaacttct aaagaaaaga gattggtttg cctcaccgtt ctgcaggctg |
| 12541 | tactggaagc atggcaccag catctatttc tcgtgacggc ctcaggctgc tcccactctg |
| 12601 | gcagaaggga aggagggtct gtctgtgcag agaccacaga gatcacacgg caagagaggg |
| 12661 | agcaaggggg agggggagtg atggagcttc caagctcttt ttaacaacca gctctccggg |
| 12721 | aactaataga gggggaactt gctaaccccg tctccttggg acagcattga tgtgttcatg |
| 12781 | atggatccac ctccatgacc caaacacctc tcaagaggcc caacctccca cagtgggggt |
| 12841 | gaaatttcaa tgtgaggttt gaaggggtca aacatctcaa ctaaagtagt cgtatcctca |
| 12901 | gcacgttcta tggttactat gagagctata actgaaaaag caggagaaag ctgggtctcc |
| 12961 | tgccatctgg gtgcttgtcc taaagagatg ttttatgtgg ttacctgtca atcaagaaat |
| 13021 | gcgagacaat tcataaagag gaactgctaa gattagcttc ttattggtgt ctcatcttct |
| 13081 | tccaggtaac ccccgacacc tgcacattct gattgggacc tcagtggtca tcatcctctt |
| 13141 | catcctcctc ttctttctcc ttcatcgctg gtgctccaac aaaaaaagta agtctcacga |
| 13201 | agcagaggcc agagagctca gggccatgtg gggaagcagg atgggagcac tcaggtgtgt |
| 13261 | gttcctcaca aacaggatgg tccctggccc aaggcagcag ccacagaggc aggactttct |
| 13321 | agagagggca ccagactccc tgcccctgcc ttcaactcac agaccgttgc ctgattctga |
| 13381 | actgtatcct catgtcccct gcagccactc acatccagga gaaggttcca tgacaggcag |
| 13441 | aaagtgggag acagaatcaa tgggatggga actcagagct attcatggga tgggtccttg |
| 13501 | agctcagaga gatagaatgt ctgagtctgc tgttggcaac tgagggacct cagccaccta |
| 13561 | tggtctcccc ctgtatgttg gtatctgctt atgaaatgag gacccagaag tgccctccga |
| 13621 | gctgttttgt tgacttccgt ctcctacaga tgctgcggta atggaccaag agtctgcagg |
| 13681 | gaacagaaca gcgaatagcg aggtaggtac tcctcggccc gggctcgtgg ctactgttat |
| 13741 | tcccaaagag tcctggaaaa tgtgagcacc ctccctcact cagcatttcc ctctctccag |
| 13801 | gactctgatg aacaagaccc tcaggaggtg acatacacac agttgaatca ctgcgttttc |
| 13861 | acacagagaa aaatcactcg cccttctcag aggcccaaga cacccccaac agatatcatc |
| 13921 | gtgtacgcgg aacttccaaa tgctgagtcc agatccaaag ttgtctcctg cccatgagca |
| 13981 | ccacagtcag gccttgaggg cgtcttctag ggagacaaca gccctgtctc aaaaccgggt |
| 14041 | tgccagctcc catgtaccag cagctggaat ctgaaggcat gagtctgcat cttagggcat |
| 14101 | cgctcttcct cacaccacaa atctgaatgt gcctctcact tgcttacaaa tgtctaaggt |
| 14161 | ccccactgcc tgctggagaa aaaacacact cctttgctta gcccacagtt ctccatttca |
| 14221 | cttgacccct gcccacctct ccaacctaac tggcttactt cctagtctac ttgaggctgc |
| 14281 | aatcacactg aggaactcac aattccaaac atacaagagg ctccctctta acgcagcact |
| 14341 | tagacacgtg ttgttccacc ttccctcatg ctgttccacc tcccctcaga ctagctttca |
| 14401 | gtcttctgtc agcagtaaaa cttatatatt ttttaaaata acttcaatgt agttttccat |
| 14461 | ccttcaaata aacatgtctg cccccatg. |

In some embodiments, the KIR2DL2 gene has the consensus sequence: gagcacccactgggcctcatgcaaggtagaaagagcctgcgtacgtcaccctcccatgatgtggtcaacatgtaaac tgcatgggcagggcgccaaataacatcctgtgcgctgctgagctgagctggggCGCGGCCGCCTGTCTG CACAGACAGCACCATGTCGCTCATGGTCGTCAGCATGGCGTGTGTTGgtgagtcct ggaagggaatcgagggagggagtgcggggatggagatcggggcccagagttggagatataggcctggaagtgg agttatgggcctagagatggagtgatgggcctagaagtggagatctgggcctggagtggagatatgggcctggaggt tgagatatgggcctgcagtagagatatgggcttgtagtggagacatgggcctggagatggagatatgggcctggaga tggagatatgggcctgcagtagagataggggcctggagtggagatatgggcctggagtggagatatgggcctgaag tggagatatgggcctggaggtggagatatgggcctggaggtggagatatgggcctggagtggagatatgggtctgga ggtggagatacgggcctgcagtagagatatgggcctggagtggagatatgggccaggagtggagttatgggcctag aggtggatatctgggcctggagtggagatatgggcctaggaaggagatatgggcctgggtgtggagatatgggactg gagaggtgatatgggcctggagtggagatatgggcttagggtggagttctgggcctggggcggagatatgggactgg attggagataggggcctagggtggagatctgagcctggattggcgatatgggcctagggtggaaatatcagcctgga gtggagatatgggcttggggtggggatatgggcctggaaactgggtctctgcacagccgacagccctgttcttgggtgc aggtaggcactgagggtgagtttaacttcagcccaggaagggcctggctgccaagactcacagcccagtgggggc agcaagggagggctggttcgcctgcagatggatcgtccatcatgatctttctttccagGGTTCTTCTTGCTGC AGGGGGCCTGGCCACATGAGGgtgagtccttctccaaaccttcgggtgtcatctccccacataagagg attttcctgaaacaggagggaagtcctgtcggggagtctctcataaactaggaagagaggaccctggggtgctcagc ccacatttctgacctcgcctccctggcctctcaaccccttggcagagtcaagttctgtggggaccagggttagactgggg tgctcaaagctggggtgtgtggttgggaagtggtaggaacagcagatcctctgaggacaaaggtgttactcacacact tcagcgtttccatgatggtaggggctgcagtgtggctgctgtcattctaccagaagaggtgggaaaccacagccatgg ccctgacattccaaatcctctgatgggggctcagttgtttattttcgttcaggcatccgctgatatccattcacaaaggacat gccctccacctcatgtctaccctgtgttgttttatgtgagtaatcttacagtatcaaaatctagtaggagtctctttactcagca cttgctcaaagttctcagctgaggcttttgttgtagggagacaccatgtctttgcgggatgggtccttccttcagccctgggc accaaggtgtgatagtagccatagaaacgtggaaagcgaggagaatcttctgagcacagggagggaggggcagtt ccacatcctcctctctaaggcggcgcctccttctccccaaggtggtcaggacaagcccttgctgtctgcctggcccagc cttgtggtgcctctaggacatgtcattcttcggtgtcactcttatcttgggtttaacaacttcagtctgtacaaggaaggtggg gtgcctgtccctgagctctacaacagaatattctggaacagccttttcatgggccctgtgacccccgcacaacagggac atacagatgtcggggttcacacacacactcccccagtgggtggtcagcacccag caaccccctggtgatcgtggtcat aggtcagagggctcctgtcttggattctccttgtcccacctcctgaatcccagagcttctggtggg catgtccttgagggtc ccatcacgcaggccctgactgtatttgtggtaaagggggattgaatacagggaaatgggtgctgtggtgggaagaata attgtccccagtgatgactacattctaatccctggagtctgtgactatgtatgttataggggaagggactgaaggggaag atggagctcatggggagacagcctggactgtcccactgggctcagtgtaatcacaagggtgcacatgaaaggagga ggaagaggggagtggggattagagcagtccagtggaagtcttcaccagctttgaaggtggaggaaggccaagagc catgaatgcaggtggcctatagaggctggaaaagtcaaggaactgattctccagagtctccagagggaacaaagcc ctgcagatgccttgattttagcccaggaaaaatagggtccaatttctgtctccagtactggaaggtgtcagtgtggtctctc ctgcttccatgcttctgataattttgtacagcagcaacaggaaaccaacactggaacccaggtcaaggacaagttaag aaacaacccaaggaaagccaggcatggtggcaggtgcatgtaatcctagcgactcaggaggctgagggcaggag aatcacttgaacccaggaaacagaggttgcagtgagcctagaccacaccacttcactccagcctgggtgaaggagt gagactctgtctccaaaattaattaattaattaaagaaaccaaagaaggagaaggttggctaccctgagatcagcaa gggtgggatgatgatgccaccaccaggctccatccacatagggaggggttgatactcctccaaccagcaccaggag ccagcctatggaagctggcaccatggagaaggcacaggcatggcaagagtggctcccagtccccaccaggaaca gggtgtgtggacactggtgcctgccttattcatcagttcatatcttctgccaaggattgcaattcatccaaaagagattgaa ccaggctgataagagcctggatgtgcagcctatcctggttcctctttcacccccacataaacagcaggaaagacatta gtgtgaaatagatacaacaccccaagagatgaggctaagcccagtgggaagggaatcagaggctactagagaca gagggacagagaagagggagggagacagatggaaggacctgcaccaggagttaagggcacagaaaagaac atgaagacacagagaggaaggagagagacagacaccagcaaggggaagcctcactcattctaggtgccatgga tgggatgataaagagagacaccttctaaactcacaacctctcttcctagGAGTCCACAGAAAACCTTCC CTCCTGGCCCACCCAGGTCGCCTGGTGAAATCAGAAGAGACAGTCATCCTGCA ATGTTGGTCAGATGTCAGGTTTGAGCACTTCCTTCTGCACAGAGAAGGGAAGTT TAAGGACACTTTGCACCTCATTGGAGAGCACCATGATGGGGTCTCCAAAGCCAA CTTCTCCATCGGTCCCATGATGCAAGACCTTGCAGGGACCTACAGATGCTACGG TTCTGTTACTCACTCCCCCTATCAGTTGTCAGCTCCCAGTGACCCTCTGGACAT CGTCATCACAGgtgagagtgtccggacattctcattgtcattgggctgcagagtgaatgatccacgacttggaa cccccaggtagttgtaaggaagatgagcttggtattcttatggagagagactgacttgctgaggtttgtaccaacagag acagagaaacaggagacacaagtacagaccaggtgtcataacggaggacagacacaggggccatacaggga gttagaaaagacagaaagagttaaaagagacagacagacagacatgtcccagagagaggtgtccctccatgctg actttgctcacagacctggcacaggttagaagtttcatttctgttttacctccacaaagtgttctctaccaggagaacccaa ggacacccatatttctgacctgagttgggccctgtggcctcaggccttgtggcacctacaggccatgtttattctgacacc tctgccttccatgtaatggagagtaaccgtcccaggatatcatggccccagaacaccaacccctgtatgctgtgtgaac ttgtggtctccagactggattctgaggctcacattccaaataaccccacatatgaaaggatcactgagaggcacagag aaaaatcaggaacaccaaaaagcaaagacataaacacacggagaatgagccagaggaaggagattgagaga ctcacagacacataaagagagagaaaagagggcagaggagtggtgagaatgatggcagggagcagagaaaa gcactaaaattagagtcctgagagagaggcacaaggacatagaaacatggagatgtggggatgaattgcagagat tccaaagagagctagagagaccgagaggcagagcaatacagatgatagatggatagatatagatagatgataaat aggtagatgatagataataggttaaagatacatagatgatgattgattgattcattaatagataatacatagagatgatg atgatgaagacagataatacgtacagatagagaggcagacagaaatcatagagagagagatgatacatacatata aataacagatgattgatggatagatagacaactgatagatacatagatgatatatagatatagatgacaggtagaga atttgtagatagg caccgaatagataaatagatagatcgacagataatagatagaaatatg cagaaagttatgaaca ggacacaacgtgagaaacttagaatttaaaaaagtaacatcaagtcaaccaatccaaggagagtcagagagaata aaacaatccaaaaacggaaaacatatctagaggtggggaagcgaggtcagagacctagagagacagagaaggt ggaagaaggaaatagacatgaagagagatggggtggagggtgagagagagagagagagagcattaggtcata gagcaggggagtgagttctcagctcaggtgaagggagctgtgacaaggaagatcctccctgaggaaaatgcctcttc tccttccagGTCTATATGAGAAACCTTCTCTCTCAGCCCAGCCGGGCCCCACGGTTC TGGCAGGAGAGAGCGTGACCTTGTCCTGCAGCTCCCGGAGCTCCTATGACATG TACCATCTATCCAGGGAGGGGGAGGCCCATGAATGTAGGTTCTCTGCAGGGCC CAAGGTCAACGGAACATTCCAGGCCGACTTTCCTCTGGGCCCTGCCACCCACG GAGGAACCTACAGATGCTTCGGCTCTTTCCGTGACTCTCCATACGAGTGGTCAA ACTCGAGTGACCCACTGCTTGTTTCTGTCACAGgtgaggaaaccccatatctgtctcatgtccta tgatcctagagccttagctgaggagcttcctgctgatgatggagataagcatggacagatgcagagagaagacgaa gcttgggtgtgagggagggatcagggcacaggatggcagacagggcacctccaaaccctcctacacggcctgcat gaaggcccgcggccagggctccaggcacacaggcagatggagaaagcggtcaggagagacccagaggaggg agactgggctcagtttgggaagatcagaggttccctcagcccctcaacattacccatttcccagaagcccatcctggcc tctcacccacacagggatgtcatcaccagcaacccctacaccctttacttttgtttgaagaaatatttattgaggataaata tacctatatagcttaccacctttaacattttttttttttttgaggcagagtctagctctgtcccctatgctgcagtgcagtggcac aatctcagctcactgcaacttccgcctcctgggttcaagtgattctcctgcctcagccacctgagtagctggtgctacagg cgcgcaccaccacgccaggctactttttgtatttttagtagagaggtggtttcaccatgttggtcgagctggtctccaactc ctgaccacgtgatccacccgcatctgcctcccaaagtgctgggattacaggcatgagccaccactcccagccacattt accatttttaagtgtaaagtctagtggtcataaatacatttataaatatatatatatatatatatgtatgtatatatatatacaca cacatatatatacatatatatatgtgtatatatatatatatatatatatatatatatatatatatatatttttttttttttttttaccctcca cccttttcttcctggcctctggaagccaccattctactctctaccttcatgagatccaccttttagctctgtatatgggtgagaa atgggaatctttgtaatgacttccagttccatccatgtggctgcaaatatcaggatgttattctttctatggatgagtagtctcc actgtgcgtatgtactacattctctctatccattcatccactgatgggcaggtaggttgactccacatcttggctactgtgaa cagtgctgcaccaatcatacgagtgcagatatcacttcgatatattgatttactttcctttggatataaacccagtagtgaa attgctggatactatgaaagttctctttttagttattcgtttgttgttttgtttttgtttttgagacagtttccctctgtgcccaggctgg agtacaagtgatgtcatcttggctcattgcaacctctgcctcctgggttcaaatgattttcctacctcagcctccctagtagc tgggattacaggtgcacgccaccatgcctggctactttttggtttttttagtatagatggggtttccccatgttggctgggctg ctctcaaactcatgacctcaactgaggtgtccgcctcggtctcccaaagtgccgggattacaggcatgatccacctcac ccaacctctttttagttctttaaaggacttccacacttttctccgtaaaggctgtactaatttacactcctaccaacagggtatt agggttctcctttctctaccactttggcaggatttcctttgcctgtcttgcagctaaaagccattttactttatttcattttattttga gatggagtttcgctcttgtcacccaggctggagtgcagtggtgcgatctcggctcaccacaacctccacctcccaggttc aagcgattctcctgcctcagcctcccgagtagctggaattacaggcacacgccaccacg cccgactaatttttgtattttt agtagagacagtgtttctccatgtgggtcagactggtctcaaactcccgaccttatgagattcacccacctcaggctctc aaagatctaggatgacagacgtgagccaccacgcccggcctaaaagccattttaatggggtgagatgaaaactcac tttgattttaatttgcgtttctctgatgatgagtgatactgagcagtttttcgtatgtggggaaatttcatgtcttttgctcctgtttca attaaatcatttgttttattgagttgtttgagcttcttatatttctagttattaatcccatctcagatg catagtttgcacatatttgct cccaatctgtgggttgtctcttcactttgttggtttatttttagcggtg cagaagttg cttagcttgaggtaatcccaatggtctat ttttg cttcgattacttgtgttttgaaggtttaaaacaaaatgtcttccttcagacaaatgtcctggagcatttccccaatattttc ttctacgtgtttcataggttcaggccttagactcacatctttaatccattttcatttgatttttgtgtatggtgacaggtagaggtg cagtttcattcctctgcatgtagatgtccaggtttccctgcactgtttattgaaaagactgtcctttcctgattgtgagttcttgg cacctttgtcaaagtccattggatgggctgggcatggtgactgacacctgcaatttcagcactttgggagcccaaggcg ggtggatcacctgaggccaggagttcaagattagtctggccgacgtgatgaaacattgtctccactaaaaatatataa attagctgagcatggtggtcagcacctataataccactactcaggagtttgaggccagagaattgattgaacccagga ggctgtggtggcagtgaaccgagattgcacctctgcactccagcctgggtgacagagcgagactccatctcaaaaga aaaaagaaaaaaacattggatgtaaatgcatggattatatttgtgttgttcattctgctccattgttctatgtgcctttcttcatg ccaacatcatgctgtcttgcttactacagctctgtaacatattttgagatcaggtagtgtgatgctcctgttttctctttatacctt gaagtctcaagacaatgggcgtcacatacaaaaattatggaaaaaaggatcccaggactcccagggcccaatatta gataacagagtgttggccatgaaccaacctcaaagatttccattgagtagaggacagacaccctcatttcctcacctct ctcctgtctcatgttctagGAAACCCTTCAAATAGTTGGCCTTCACCCACTGAACCAAGCTC TAAAACCGgtgagtacagaaccctcttatatccgcttttggaaacctggggaggtagaaaccttcgatgcagg cat tgactcagcatctcgcagctctgacattgtacgcctgtcttctaccatctccgaactccagatactccaacagcgaaagg gatctgggcccaacctagggctcagtgaaatctcttaatctctcattttatggagctgagacctcctacaagctagaaga atgattgccaatctgacatccttctcaggaaaaatgcaatgtttgttctgcctgcattcctaactggaggataaattcctgg gggcttgagagagggaagggaagggaacatctgatgagggcgaggtgttttagagaagttccacttgccaaggaat gaattactgttggtcatgaagcaaccctggctgactcagcagagcaacagccttgccgtaacagagaacggagctc atgcacgcacacttcgactcactgactcattcagccacggccccatgctcaggctgtgcagtgcggaaccttttcctatt gttgccataacaaatttccacaagattcgtgggtgaaaacaaaacggttttttaattatcttacagtgctgtagctcaaagt aggaagtgcatcttactgggctaaaatcaaggtgacagcaaggctgccttccctctgaggattccaggcaagaatctg cttctcacttgtcccagcttctaaaggctcccagttccttggctcctggtccccttcctccttcctcaaaacccacaaagact ggtcacatctcacatggcatcactcagtgccttcttccttaccacacctctttctctgaatgctgctctcccttcttccttatctttt gaaaacttggggattctattgggttcaccaagatgaaaatccctcataatctcctggaaatcatccaggatacccttgtttt aagttcagctgattagcaaccgtaattccatctacaatcttcattcctcctttccatgtaaaataacatattcacaaggtatg gaggctaggacagggacattttggggtgggacagcattctcctgccttccacaaacagtgaacaagatgcatttggcc tctgcccttgggacactgatattgcagatggttaaatgggagggcagaaaatgaatgcacaagtggatctataaatga atgatccattgggaagcatctgtgcatgaaatctattttttgtttgttcttttgtttattgagacagagttgccctctgtcttccag gctacagtgcagtgtcacgatcttggctcactgcaacctgcttctcctggattcaagtgattctcctgcctccgcctctcga gtagctgggattacaggcaactgccaccgtgcccggctaattctttttgtatattttttgtagagaggatgtttcaccacgttg gccaagcttgtctgaaactcccaacctcaagtgatccgaccgtctcagcatgccaaagtaatgggactacaggcgtg ag ccactgtgcccagccagaattcaaaatcaataatagataatg ctgagtgtatgatttcaggtgacaaagaaggtct cactattcagatatttgtgacattaatgaaaaacacggattgaacccctgaaagattgg cggaaggattttgcacacac agctgtcagccgtgaaggcacaaaggtgaaaacaatctgatgtggaaggaagaggctcttcctcaaatgctgggaa tgatgtggggagaatgacaagatgactgtggagagacggagagcacactgggtacacaggaaactaaggagga acaaggagtgtgtgtttgacactcacagccattggattcacctcggggtagccaggaatccctacatgattaatatgact gacatgaaaataagggaggctcagttg cataactggaatctaggagaccgtggaaaagg caattgccgccccactg gtgaaatgtggtgctgatttagacactaaatgaatgaagtagatggatataagataggtttgtgaggtagaatcattgac tggaaagg cttgctgggtttgattttcctacttgtttaatcctcgcttaattaatttctttctgagatttattcatcctacacataaat caatacctggcaaaggagtgacagatatatgaggggtggtggaaatgaagagacctattatagcataatatacaagt ctgtgaacggtggctcacgcctgtaacccagcactgcaggaggccaaggcgggtggatcacatgaagtcagcagtt cgagaccagcctggccaacatggtgaaaccctgtctctaggaaaaacacaaaaattagccgagcatggtggtgcat ccctgtaatcccagctcctactctggaggatgaagcaggagaatgacttcaacccaggaggtggaggttgcagtgag tggaggttgcatcactgcactccagcctgggtggcacaaggagactccgtctcaaaaaataaaaataagaaatgcat aaatataaatataatataacacacgcaaatgacaaagggacctgaattccaatcatgatttttctatttctctataattactt ctttgatcctttatcttatccattaggcaatgagcctaaaacctcttccctatttggctttctgtgagcatgagatcatatagaa aatgtgaaagtccgctgaatcctccagcacagatcctggaatagagaaagtgctctggtcatcacaaaaaaaacttg cccactcacccaaatcccccacctcacccctacttccaatcacctgtggagattcaggtagaccatggggaggtaaa cattaacactccttggagtgagtccagatcttggaatcagagatcag cgacagcactag ctcctgctcccctttcctact aattcacaggaggacaggtggtattgaagcaatagatggccgagggggtggtccttcccccagcctctcgggtagaa cagcagcctaatatgtgtctcccgagatcacaaagagcagcaggtttcacacgggcttcaacactatttcctggccgttt gacataagagaattctatttcgctttttttatcttgatttcacttttgttttctttccttggagaatgcaagttgtttgattcaagaatg ctgtggatgtagaaaccctaaagcacattcgctgtgaatcaatcccagtccagtcttcccagagaagactctaaacac ctcctggactgcacctgggcctatgccaattcctatcactcaccgtcactccagggagacagaacacacagagaata cgttacataggcaggttcattactaacagataagcagcgagtgacaacagaaacctatatttcaatgtgagccagtcc ctcaaggctcagaaaagctcctcgggacatatggagtcaccccatttgcagtgtagctgcgggaagccagaaagca gcccagcctgggttttgtaccctggagccacaggaagcactcagctaaagcactgcatgacgtcctccaggaagaa caggaagacagcccagggtgttctgagacgttcctcctgatctcaggaagttgctgtcttaggccatttttgttgctctaaa ggaacacttgagcctcggtaacttctaaagaaaagagattggtttgcctcaccgttctgcaggctgtactggaagcatg gcaccagcatctatttctcgtgacggcctcaggctgctcccactctggcagaagggaaggagggtctgtctgtgcaga gaccacagagatcacacggcaagagagggagcaagggggagggggagtgatggagcttccaagctctttttaac aaccagctctccgggaactaatagaggggggaacttgctaaccccgtctccttgggacagcattgatgtgttcatgatgg atccacctccatgacccaaacacctctcaagaggcccaacctcccacagtgggggtgaaatttcaatgtgaggtttga aggggtcaaacatctcaactaaagtagtcgtatcctcagcacgttctatggttactatgagagctataactgaaaaagc aggagaaagctgggtctcctgccatctgggtgcttgtcctaaagagatgttttatgtggttacctgtcaatcaagaaatgc gagacaattcataaagaggaactgctaagattagcttcttattggtgtctcatcttcttccagGTAACCCCCGACA CCTGCACATTCTGATTGGGACCTCAGTGGTCATCATCCTCTTCATCCTCCTCTTC TTTCTCCTTCATCGCTGGTGCTCCAACAAAAAAAgtaagtctcacgaagcagaggccagaga gctcagggccatgtggggaagcaggatgggagcactcaggtgtgtgttcctcacaaacaggatggtccctggccca aggcagcagccacagaggcaggactttctagagagggcaccagactccctgcccctg ccttcaactcacagaccgt tgcctgattctgaactgtatcctcatgtcccctgcagccactcacatccaggagaaggttccatgacagg cagaaagtg ggagacagaatcaatgggatgggaactcagagctattcatgggatgggtccttgagctcagagagatagaatgtctg agtctgctgttggcaactgagggacctcagccacctatggtctccccctgtatgttggtatctg cttatgaaatgaggacc cagaagtgccctccgagctgttttgttgacttccgtctcctacagATGCTGCGGTAATGGACCAAGAGT CTGCAGGGAACAGAACAGCGAATAGCGAGgtaggtactcctcggcccgggctcgtggctactgtt attcccaaagagtcctggaaaatgtgagcaccctccctcactcagcatttccctctctccagGACTCTGATGAA CAAGACCCTCAGGAGGTGACATACACACAGTTGAATCACTGCGTTTTCACACAG AGAAAAATCACTCGCCCTTCTCAGAGGCCCAAGACACCCCCAACAGATATCATC GTGTACACGGAACTTCCAAATGCTGAGTCCAGATCCAAAGTTGTCTCCTGCCCA TGAGCACCACAGTCAGGCCTTGAGGGCGTCTTCTAGGGAGACAACAGCCCTG TCTCAAAACCGGGTTGCCAGCTCCCATGTACCAGCAGCTGGAATCTGAAGGC ATGAGTCTGCATCTTAGGGCATCGCTCTTCCTCACACCACAAATCTGAATGTG CCTCTCACTTGCTTACAAATGTCTAAGGTCCCCACTGCCTGCTGGAGAAAAAA CACACTCCTTTGCTTAGCCCACAGTTCTCCATTTCACTTGACCCCTGCCCACCT CTCCAACCTAACTGGCTTACTTCCTAGTCTACTTGAGGCTGCAATCACACTGA GGAACTCACAATTCCAAACATACAAGAGGCTCCCTCTTAACGCAGCACTTAGA CACGTGTTGTTCCACCTTCCCTCATGCTGTTCCACCTCCCCTCAGACTAGCTTT CAGTCTTCTGTCAGCAGTAAAACTTATATATTTTTTAAAATAACTTCAATGTAGT TTTCCATCCTTCAAATAAACATGTCTGCCCCCATG (SEQ ID NO:3), comprising a promoter region (italicized), 5′UTR and 3′UTR (upper case and bold), EXONS (upper case), and introns (lower case)

In some embodiments, the protein coding sequence of the KIR2DL2 gene has the consensus sequence:

CGCGGCCGCCTGTCTGCACAGACAGCACCatgtcgctcatggtcgtcagcatggcgtgtgttggg ttcttcttgctgcagggggcctggccaCATGAGGGAGTCCACAGAAAACCTTCCCTCCTGGCC CACCCAGGTCGCCTGGTGAAATCAGAAGAGACAGTCATCCTGCAATGTTGGTCA GATGTCAGGTTTGAGCACTTCCTTCTGCACAGAGAAGGGAAGTTTAAGGACACT TTGCACCTCATTGGAGAGCACCATGATGGGGTCTCCAAAGCCAACTTCTCCATC GGTCCCATGATGCAAGACCTTGCAGGGACCTACAGATGCTACGGTTCTGTTACT CACTCCCCCTATCAGTTGTCAGCTCCCAGTGACCCTCTGGACATCGTCATCACA GGTCTATATGAGAAACCTTCTCTCTCAGCCCAGCCGGGCCCCACGGTTCTGGC AGGAGAGAGCGTGACCTTGTCCTGCAGCTCCCGGAGCTCCTATGACATGTACC ATCTATCCAGGGAGGGGGAGGCCCATGAATGTAGGTTCTCTGCAGGGCCCAAG GTCAACGGAACATTCCAGGCCGACTTTCCTCTGGGCCCTGCCACCCACGGAGG AACCTACAGATGCTTCGGCTCTTTCCGTGACTCTCCATACGAGTGGTCAAACTC GAGTGACCCACTGCTTGTTTCTGTCACAGGAAACCCTTCAAATAGTTGGCCTTC ACCCACTGAACCAAGCTCTAAAACCGGTAACCCCCGACACCTGCACATTCTGAT TGGGACCTCAGTGGTCATCATCCTCTTCATCCTCCTCTTCTTTCTCCTTCATCGC TGGTGCTCCAACAAAAAAAATGCTGCGGTAATGGACCAAGAGTCTGCAGGGAA CAGAACAGCGAATAGCGAGGACTCTGATGAACAAGACCCTCAGGAGGTGACAT ACACACAGTTGAATCACTGCGTTTTCACACAGAGAAAAATCACTCGCCCTTCTCA GAGGCCCAAGACACCCCCAACAGATATCATCGTGTACACGGAACTTCCAAATGC TGAGTCCAGATCCAAAGTTGTCTCCTGCCCATGAGCACCACAGTCAGGCCTTG AGGGCGTCTTCTAGGGAGACAACAGCCCTGTCTCAAAACCGGGTTGCCAGCT CCCATGTACCAGCAGCTGGAATCTGAAGGCATGAGTCTGCATCTTAGGGCATC GCTCTTCCTCACACCACAAATCTGAATGTGCCTCTCACTTGCTTACAAATGTCT AAGGTCCCCACTGCCTGCTGGAGAAAAAACACACTCCTTTGCTTAGCCCACAG TTCTCCATTTCACTTGACCCCTGCCCACCTCTCCAACCTAACTGGCTTACTTCC TAGTCTACTTGAGGCTGCAATCACACTGAGGAACTCACAATTCCAAACATACA AGAGGCTCCCTCTTAACGCAGCACTTAGACACGTGTTGTTCCACCTTCCCTCA TGCTGTTCCACCTCCCCTCAGACTAGCTTTCAGTCTTCTGTCAGCAGTAAAACT TATATATTTTTTAAAATAACTTCAATGTAGTTTTCCATCCTTCAAATAAACATGT CTGCCCCCATG (SEQ ID NO:4), comprising a 5′UTR and 3′UTR (upper case and bold) and a signal peptide (lower case).

Therefore, disclosed herein are gRNA and cRNA that can be used to ablate KIR2DL2 expression in immune effector cells. Note that differences between a gRNA and a crRNA are based on Cas specificities. While gRNAs are designed to couple with a tracer RNA (tracRNA) to pair with the Cas9, the crRNA are designed to form a ribonucleoprotein (RNP) complex with the Cas12 with no need of tracRNA.

In some embodiments, the gRNA and cRNA can be used to ablate KIR2DL2 expression in immune effector cells are provided in Table 1.

TABLE 1
KIR2DL2 guide RNA (gRNA*) and CRISPR RNA (crRNA*)
Cas9

| | | | | Specificity | Efficiency |
|---|---|---|---|---|---|
| | | | | Score (Off | Score (On |
| Region targeted | Strand | Sequence | PAM | target score) | target score) |
| Promoter | 1 | ctcttgagcgagcacccact (SEQ ID NO: 5) | ggg | 15.9815022 | 46.26123025 |
| | 1 | gctcttgagcgagcacccac (SEQ ID NO: 6) | tgg | 15.9325168 | 38.82200489 |
| | −1 | agtgggtgctcgctcaagag (SEQ ID NO: 7) | cgg | 15.8645005 | 61.65603932 |
| | −1 | gctcgctcaagagcggaaca (SEQ ID NO: 8) | tgg | 15.6348821 | 59.891163 |
| | −1 | taccttgcatgaggcccagt (SEQ ID NO: 9) | ggg | 15.3838426 | 47.99589073 |
| | 1 | tggtcaacatgtaaactgca (SEQ ID NO: 10) | tgg | 14.8022274 | 60.25262163 |
| | 1 | tgtgcgctgctgagctgagc (SEQ ID NO: 11) | tgg | 14.58646 | 26.51616174 |
| | 1 | ggtcaacatgtaaactgcat (SEQ ID NO: 12) | ggg | 14.1942045 | 66.69398072 |
| | −1 | catgggagggtgacgtacgc (SEQ ID NO: 13) | agg | 14.1725166 | 59.57214176 |
| | −1 | atgttgaccacatcatggga (SEQ ID NO: 14) | ggg | 13.9805565 | 67.58548647 |
| | −1 | tttacatgttgaccacatca (SEQ ID NO: 15) | tgg | 13.9640537 | 60.21717187 |
| | 1 | acatgtaaactgcatgggca (SEQ ID NO: 16) | ggg | 13.8728239 | 59.4772864 |
| | −1 | gcagcgcacaggatgttatt (SEQ ID NO: 17) | tgg | 13.8464645 | 38.09327197 |
| | 1 | tgcgctgctgagctgagctg (SEQ ID NO: 18) | ggg | 13.7588893 | 52.71880216 |
| | 1 | aacatgtaaactgcatgggc (SEQ ID NO: 19) | agg | 13.4718305 | 46.46066649 |
| | −1 | agctcagctcagcagcgcac (SEQ ID NO: 20) | agg | 13.2454872 | 48.30097161 |
| | −1 | ttacatgttgaccacatcat (SEQ ID NO: 21) | ggg | 12.7849142 | 58.65244492 |
| | 1 | gtgcgctgctgagctgagct (SEQ ID NO: 22) | ggg | 12.0932581 | 29.66669834 |
| | 1 | tacgtcaccctcccatgatg (SEQ ID NO: 23) | tgg | 12.0547498 | 57.3970135 |
| | −1 | catgttgaccacatcatggg (SEQ ID NO: 24) | agg | 12.0445433 | 60.83312873 |
| | −1 | ggctctttctaccttgcatg (SEQ ID NO: 25) | agg | 11.895067 | 56.77308491 |
| | −1 | ctaccttgcatgaggcccag (SEQ ID NO: 26) | tgg | 11.6270243 | 63.92002251 |
| | 1 | cacccactgggcctcatgca (SEQ ID NO: 27) | agg | 11.622461 | 55.11316701 |
| 5′UTR + Signal | 1 | agacagcaccatgtcgctca (SEQ ID NO: 28) | tgg | 65.7857251 | 61.74680156 |
| peptide | −1 | atggtgctgtctgtgcagac (SEQ ID NO: 29) | agg | 50.7755911 | 61.01765228 |
| | −1 | gtgctgtctgtgcagacagg (SEQ ID NO: 30) | cgg | 42.4198704 | 65.8510863 |
| | −1 | gctgacgaccatgagcgaca (SEQ ID NO: 31) | tgg | 17.4180397 | 68.97222201 |
| | 1 | gtcgctcatggtcgtcagca (SEQ ID NO: 32) | tgg | 15.8075075 | 48.88926217 |
| Signal peptide + | 1 | cagggggcctggccacatga (SEQ ID NO: 33) | ggg | 25.6759978 | 53.42004189 |
| Exon 2 | −1 | ggactcaccctcatgtggcc (SEQ ID NO: 34) | agg | 24.7318646 | 50.06679646 |
| | 1 | cagggttcttcttgctgcag (SEQ ID NO: 35) | ggg | 16.7082395 | 47.66879684 |
| | 1 | ccagggttcttcttgctgca (SEQ ID NO: 36) | ggg | 16.5530134 | 42.85183717 |
| | 1 | tccagggttcttcttgctgc (SEQ ID NO: 37) | agg | 16.4294081 | 25.66544124 |
| | 1 | tcttcttgctgcagggggcc (SEQ ID NO: 38) | tgg | 16.3937902 | 23.97461357 |
| | 1 | agggttcttcttgctgcagg (SEQ ID NO: 39) | ggg | 16.3044063 | 53.09881316 |
| | 1 | gcagggggcctggccacatg (SEQ ID NO: 40) | agg | 15.8202174 | 47.71767808 |
| | −1 | ccctgcagcaagaagaaccc (SEQ ID NO: 41) | tgg | 15.2116359 | 54.97024219 |
| Exon 3 | −1 | cctgcaaggtcttgcatcat (SEQ ID NO: 42) | ggg | 79.5879626 | 41.94420172 |
| | 1 | cccatgatgcaagaccttgc (SEQ ID NO: 43) | agg | 72.4619282 | 47.76943418 |
| | −1 | ccctgcaaggtcttgcatca (SEQ ID NO: 44) | tgg | 72.4330928 | 50.76857508 |
| | 1 | ccatgatgcaagaccttgca (SEQ ID NO: 45) | ggg | 68.7469834 | 60.94960431 |
| | 1 | aaggacactttgcacctcat (SEQ ID NO: 46) | tgg | 67.2663317 | 49.30177556 |
| | −1 | gtcttgcatcatgggaccga (SEQ ID NO: 47) | tgg | 54.8096904 | 54.75667126 |
| | 1 | gcacagagaagggaagttta (SEQ ID NO: 48) | agg | 48.9411384 | 37.28340475 |
| | 1 | accgatggagaagttggctt (SEQ ID NO: 49) | tgg | 40.5434549 | 35.55658063 |
| | −1 | tctgatttcaccaggcgacc (SEQ ID NO: 50) | tgg | 36.9267672 | 39.34508609 |
| | −1 | ctgtgatgacgatgtccaga (SEQ ID NO: 51) | ggg | 36.8255359 | 64.83990028 |
| | 1 | catcatggtgctctccaatg (SEQ ID NO: 52) | agg | 31.0596287 | 67.52356313 |
| | 1 | cctggcccacccaggtcgcc (SEQ ID NO: 53) | tgg | 30.3422942 | 40.4554791 |
| | 1 | tgcaatgttggtcagatgtc (SEQ ID NO: 54) | agg | 28.852938 | 42.43970536 |
| | −1 | ctgatttcaccaggcgacct (SEQ ID NO: 55) | ggg | 28.2209907 | 50.80004796 |
| | 1 | tcattggagagcaccatgat (SEQ ID NO: 56) | ggg | 25.536319 | 54.0081352 |
| | 1 | ctcattggagagcaccatga (SEQ ID NO: 57) | tgg | 24.7282726 | 54.6636991 |
| | 1 | cattggagagcaccatgatg (SEQ ID NO: 58) | ggg | 22.3248805 | 64.51568239 |
| | −1 | tttcaccaggcgacctgggt (SEQ ID NO: 59) | ggg | 21.945129 | 56.88886502 |
| | −1 | atttcaccaggcgacctggg (SEQ ID NO: 60) | tgg | 21.3848745 | 59.80225741 |
| | 1 | tccaaagccaacttctccat (SEQ ID NO: 61) | cgg | 20.3315154 | 54.61351716 |
| | 1 | acttccttctgcacagagaa (SEQ ID NO: 62) | ggg | 20.0787861 | 54.50715496 |
| | −1 | acttcccttctctgtgcaga (SEQ ID NO: 63) | agg | 19.0230959 | 53.39949127 |
| | −1 | ccaggcgacctgggtgggcc (SEQ ID NO: 64) | agg | 18.1730721 | 41.63264924 |
| | −1 | gacgatgtccagagggtcac (SEQ ID NO: 65) | tgg | 17.8540881 | 39.43995673 |
| | −1 | tggctttggagaccccatca (SEQ ID NO: 66) | tgg | 17.6135951 | 57.28898297 |
| | −1 | agcatctgtaggtccctgca (SEQ ID NO: 67) | agg | 17.5270305 | 61.14244139 |
| | −1 | catgggaccgatggagaagt (SEQ ID NO: 68) | tgg | 17.5075974 | 47.77644971 |
| | −1 | gggagctgacaactgatagg (SEQ ID NO: 69) | ggg | 17.1716987 | 66.56681924 |
| | −1 | ctgtctcttctgatttcacc (SEQ ID NO: 70) | agg | 17.0018113 | 52.48561839 |
| | −1 | ctgggagctgacaactgata (SEQ ID NO: 71) | ggg | 16.7225486 | 44.13169675 |
| | −1 | tgggagctgacaactgatag (SEQ ID NO: 72) | ggg | 16.6426956 | 58.90662026 |
| | −1 | aacagaaccgtagcatctgt (SEQ ID NO: 73) | agg | 15.5732951 | 66.08963393 |
| | 1 | actgggagctgacaactgat (SEQ ID NO: 74) | agg | 14.7844691 | 50.36680662 |
| | 1 | gtcagctcccagtgaccctc (SEQ ID NO: 75) | tgg | 13.9887563 | 42.25362939 |
| | −1 | acgatgtccagagggtcact (SEQ ID NO: 76) | ggg | 13.7921026 | 60.75050183 |
| | −1 | ggcgacctgggtgggccagg (SEQ ID NO: 77) | agg | 13.6590717 | 57.639565 |
| | −1 | gcgacctgggtgggccagga (SEQ ID NO: 78) | ggg | 13.2100431 | 50.05340313 |
| | 1 | tgacatctgaccaacattgc (SEQ ID NO: 79) | agg | 12.8591707 | 41.43631776 |
| | 1 | gcagggacctacagatgcta (SEQ ID NO: 80) | cgg | 12.7977995 | 59.98223405 |
| | 1 | agacagtcatcctgcaatgt (SEQ ID NO: 81) | tgg | 12.6543101 | 51.27120381 |
| | 1 | cacttccttctgcacagaga (SEQ ID NO: 82) | agg | 12.553479 | 47.24932091 |
| | 1 | ccacagaaaaccttccctcc (SEQ ID NO: 83) | tgg | 12.179199 | 51.892235 |
| | 1 | ccttccctcctggcccaccc (SEQ ID NO: 84) | agg | 11.4972374 | 53.63053228 |
| | −1 | ccaggagggaaggttttctg (SEQ ID NO: 85) | tgg | 11.2593255 | 54.0490957 |
| | −1 | cctgggtgggccaggaggga (SEQ ID NO: 86) | agg | 10.6308453 | 45.10008683 |
| Exon 4 | −1 | ctgcagagaacctacattca (SEQ ID NO: 87) | tgg | 58.126023 | 41.77042991 |
| | 1 | agggggaggcccatgaatgt (SEQ ID NO: 88) | agg | 57.5984019 | 54.91868055 |
| | −1 | tgcagagaacctacattcat (SEQ ID NO: 89) | ggg | 53.8049699 | 47.1931636 |
| | 1 | catgaatgtaggttctctgc (SEQ ID NO: 90) | agg | 48.8037638 | 47.15855912 |
| | 1 | atgaatgtaggttctctgca (SEQ ID NO: 91) | ggg | 42.9541779 | 63.70794223 |
| | 1 | tccgtgactctccatacgag (SEQ ID NO: 92) | tgg | 42.3518877 | 64.95530799 |
| | −1 | gctctctcctgccagaaccg (SEQ ID NO: 93) | tgg | 39.9122374 | 53.48535523 |
| | 1 | agcatctgtaggttcctccg (SEQ ID NO: 94) | tgg | 32.9490905 | 60.90621662 |
| | 1 | taggttctctgcagggccca (SEQ ID NO: 95) | agg | 31.8896396 | 56.55116007 |
| | −1 | ctcgagtttgaccactcgta (SEQ ID NO: 96) | tgg | 31.0199135 | 56.19377776 |
| | −1 | accactcgtatggagagtca (SEQ ID NO: 97) | cgg | 29.8382482 | 67.75748604 |
| | −1 | ctctctcctgccagaaccgt (SEQ ID NO: 98) | ggg | 28.317125 | 40.78289164 |
| | −1 | ggcccagaggaaagtcggcc (SEQ ID NO: 99) | tgg | 27.118728 | 35.48158427 |
| | 1 | ctggaatgttccgttgacct (SEQ ID NO: 100) | tgg | 26.6891718 | 39.22415654 |
| | −1 | tggaatgttccgttgacctt (SEQ ID NO: 101) | ggg | 26.2257382 | 43.51741671 |
| | 1 | tctgcagggcccaaggtcaa (SEQ ID NO: 102) | cgg | 25.5217031 | 58.68215106 |
| | −1 | gcatctgtaggttcctccgt (SEQ ID NO: 103) | ggg | 23.7641164 | 64.09139455 |
| | 1 | ggatagatggtacatgtcat (SEQ ID NO: 104) | agg | 22.5719786 | 58.79313198 |
| | 1 | caaggtcaacggaacattcc (SEQ ID NO: 105) | agg | 22.5410529 | 45.79626069 |
| | 1 | ccgggccccacggttctggc (SEQ ID NO: 106) | agg | 22.1712727 | 46.55746509 |
| | 1 | atgacatgtaccatctatcc (SEQ ID NO: 107) | agg | 22.0305575 | 44.75201749 |
| | −1 | tctctcctgccagaaccgtg (SEQ ID NO: 108) | ggg | 20.8300787 | 67.22642591 |
| | 1 | ttcatgggcctccccctccc (SEQ ID NO: 109) | tgg | 20.4729882 | 39.96596612 |
| | 1 | tgtaccatctatccagggag (SEQ ID NO: 110) | ggg | 20.034197 | 58.18580496 |
| | 1 | ataggagctccgggagctgc (SEQ ID NO: 111) | agg | 18.8988426 | 49.6265444 |
| | 1 | attccaggccgactttcctc (SEQ ID NO: 112) | tgg | 18.831583 | 31.78584288 |
| | 1 | taggttcctccgtgggtggc (SEQ ID NO: 113) | agg | 18.6401946 | 33.90580348 |
| | 1 | ttccaggccgactttcctct (SEQ ID NO: 114) | ggg | 18.3037756 | 51.86413845 |
| | 1 | atgtaccatctatccaggga (SEQ ID NO: 115) | ggg | 17.9123846 | 65.85919053 |
| | 1 | tgacatgtaccatctatcca (SEQ ID NO: 116) | ggg | 17.6685576 | 64.17145284 |
| | −1 | ccagaaccgtggggcccggc (SEQ ID NO: 117) | tgg | 17.5233952 | 26.25436916 |
| | 1 | cctgccagaaccgtggggcc (SEQ ID NO: 118) | cgg | 17.4086492 | 46.57815807 |
| | −1 | cagaaccgtggggcccggct (SEQ ID NO: 119) | ggg | 17.2023021 | 40.47921169 |
| | 1 | ggaggaacctacagatgctt (SEQ ID NO: 120) | cgg | 17.1023595 | 52.37081136 |
| | 1 | ccagccgggccccacggttc (SEQ ID NO: 121) | tgg | 16.4944144 | 31.16041254 |
| | 1 | gtaccatctatccagggagg (SEQ ID NO: 122) | ggg | 16.2348456 | 59.07845567 |
| | −1 | gtacatgtcataggagctcc (SEQ ID NO: 123) | ggg | 15.844385 | 54.15898367 |
| | 1 | catgtaccatctatccaggg (SEQ ID NO: 124) | agg | 15.7311139 | 71.77109475 |
| | 1 | ccatctatccagggaggggg (SEQ ID NO: 125) | agg | 15.6443179 | 45.35911519 |
| | −1 | ggtacatgtcataggagctc (SEQ ID NO: 126) | cgg | 15.5683706 | 46.55891931 |
| | −1 | ggcagggcccagaggaaagt (SEQ ID NO: 127) | cgg | 15.4697357 | 49.6486146 |
| | −1 | cctccccctccctggataga (SEQ ID NO: 128) | tgg | 15.4331486 | 37.06286031 |
| | −1 | tctgtaggttcctccgtggg (SEQ ID NO: 129) | tgg | 14.5487998 | 63.6725736 |
| | −1 | ctgtgacagaaacaagcagt (SEQ ID NO: 130) | ggg | 14.167692 | 70.81083421 |
| | −1 | gctccgggagctgcaggaca (SEQ ID NO: 131) | agg | 13.2586199 | 56.31871721 |
| | 1 | tgaccttgtcctgcagctcc (SEQ ID NO: 132) | cgg | 13.1335213 | 44.62611872 |
| | −1 | aggttcctccgtgggtggca (SEQ ID NO: 133) | ggg | 12.996601 | 51.90925589 |
| | 1 | ctcagcccagccgggcccca (SEQ ID NO: 134) | cgg | 12.6112714 | 45.24662974 |
| | 1 | ctgggccctgccacccacgg (SEQ ID NO: 135) | agg | 12.2718516 | 60.36188811 |
| | 1 | ccttctctctcagcccagcc (SEQ ID NO: 136) | ggg | 12.0416884 | 50.40044061 |
| | −1 | gaaagagccgaagcatctgt (SEQ ID NO: 137) | agg | 11.8256907 | 64.81372513 |
| | 1 | accttctctctcagcccagc (SEQ ID NO: 138) | cgg | 11.5867593 | 40.49696239 |
| | −1 | cccggctgggctgagagaga (SEQ ID NO: 139) | agg | 10.6934148 | 48.09196456 |
| | −1 | ccgtgggtggcagggcccag (SEQ ID NO: 140) | agg | 10.0379832 | 59.97182871 |
| | 1 | cctctgggccctgccaccca (SEQ ID NO: 141) | cgg | 9.6833026 | 49.04004993 |
| Exon 5 | −1 | tactcaccggttttagagct (SEQ ID NO: 142) | tgg | 57.852457 | 35.49280109 |
| | 1 | actgaaccaagctctaaaac (SEQ ID NO: 143) | cgg | 48.7345562 | 54.98912793 |
| | −1 | gttttagagcttggttcagt (SEQ ID NO: 144) | ggg | 37.0092113 | 55.32743764 |
| | −1 | ggttttagagcttggttcag (SEQ ID NO: 145) | tgg | 36.4002409 | 55.47355384 |
| | −1 | ggtgaaggccaactatttga (SEQ ID NO: 146) | agg | 20.7173391 | 43.55714515 |
| | 1 | taggaaacccttcaaatagt (SEQ ID NO: 147) | tgg | 20.5263725 | 42.8174115 |
| | −1 | gtgaaggccaactatttgaa (SEQ ID NO: 148) | ggg | 19.9184363 | 51.3711373 |
| | −1 | gagcttggttcagtgggtga (SEQ ID NO: 149) | agg | 14.748832 | 55.63346666 |
| Exon 6 | −1 | tcagaatgtgcaggtgtcgg (SEQ ID NO: 150) | ggg | 58.3772674 | 58.97985736 |
| | −1 | atcagaatgtgcaggtgtcg (SEQ ID NO: 151) | ggg | 55.9818205 | 54.73894422 |
| | 1 | cgacacctgcacattctgat (SEQ ID NO: 152) | tgg | 41.7051956 | 45.76563799 |
| | −1 | tgcaggtgtcgggggttacc (SEQ ID NO: 153) | tgg | 41.1213682 | 41.27410693 |
| | −1 | caatcagaatgtgcaggtgt (SEQ ID NO: 154) | cgg | 38.0757244 | 53.47960664 |
| | −1 | aatcagaatgtgcaggtgtc (SEQ ID NO: 155) | ggg | 36.8116094 | 41.65166937 |
| | 1 | aggtcccaatcagaatgtgc (SEQ ID NO: 156) | agg | 35.8407269 | 60.52618338 |
| | 1 | gacacctgcacattctgatt (SEQ ID NO: 157) | ggg | 31.2544152 | 39.39880007 |
| | −1 | tgttggagcaccagcgatga (SEQ ID NO: 158) | agg | 22.967965 | 50.79660146 |
| | 1 | cattctgattgggacctcag (SEQ ID NO: 159) | tgg | 21.2419253 | 67.83334055 |
| | −1 | gaagaggatgatgaccactg (SEQ ID NO: 160) | agg | 18.7676197 | 77.02928766 |
| | −1 | gtgagacttactttttttgt (SEQ ID NO: 161) | tgg | 17.8896759 | 35.81269774 |
| | 1 | tcttctttctccttcatcgc (SEQ ID NO: 162) | tgg | 16.0977219 | 41.84089734 |
| | −1 | gaaagaagaggaggatgaag (SEQ ID NO: 163) | agg | 14.9946626 | 56.87278435 |
| | −1 | agcgatgaaggagaaagaag (SEQ ID NO: 164) | agg | 12.9288675 | 58.06787307 |
| | 1 | gatgaaggagaaagaagagg (SEQ ID NO: 165) | agg | 9.0025407 | 66.1476459 |
| Exon 7 | 1 | tcctacagatgctgcggtaa (SEQ ID NO: 166) | tgg | 39.0868305 | 42.2970887 |
| | 1 | tccattaccgcagcatctgt (SEQ ID NO: 167) | agg | 38.9014202 | 58.3166372 |
| | 1 | taatggaccaagagtctgca (SEQ ID NO: 168) | ggg | 36.3621703 | 63.23445862 |
| | 1 | gaacagaacagcgaatagcg (SEQ ID NO: 169) | agg | 32.2502225 | 67.08996298 |
| | 1 | agaacagcgaatagcgaggt (SEQ ID NO: 170) | agg | 31.2359366 | 63.3723398 |
| | 1 | gtaatggaccaagagtctgc (SEQ ID NO: 171) | agg | 29.6230987 | 56.94666444 |
| | −1 | ttctgttccctgcagactct (SEQ ID NO: 172) | tgg | 25.0386945 | 36.77304996 |
| Exon 8 | 1 | aacagatatcatcgtgtaca (SEQ ID NO: 173) | cgg | 45.2890654 | 62.71699919 |
| | −1 | gtacacgatgatatctgttg (SEQ ID NO: 174) | ggg | 44.9529219 | 61.98329108 |
| | −1 | gtgtacacgatgatatctgt (SEQ ID NO: 175) | tgg | 44.2078237 | 57.17566505 |
| | −1 | tgtacacgatgatatctgtt (SEQ ID NO: 176) | ggg | 43.5868555 | 42.60355046 |
| | −1 | tacacgatgatatctgttgg (SEQ ID NO: 177) | ggg | 43.2709289 | 72.67309996 |
| | −1 | ttggatctggactcagcatt (SEQ ID NO: 178) | tgg | 26.7862203 | 36.78849828 |
| | −1 | tgtgtgtatgtcacctcctg (SEQ ID NO: 179) | agg | 26.5688904 | 56.3411972 |
| | −1 | gtgtgtatgtcacctcctga (SEQ ID NO: 180) | ggg | 26.0070899 | 61.3014034 |
| | −1 | gcaggagacaactttggatc (SEQ ID NO: 181) | tgg | 18.3112446 | 44.99853999 |
| | −1 | tcatgggcaggagacaactt (SEQ ID NO: 182) | tgg | 18.2856344 | 58.0617284 |
| | −1 | atatctgttgggggtgtctt (SEQ ID NO: 183) | ggg | 17.9587785 | 34.80286512 |
| | −1 | gatatctgttgggggtgtct (SEQ ID NO: 184) | tgg | 17.5950576 | 33.17325643 |
| | −1 | ggtcttgttcatcagagtcc (SEQ ID NO: 185) | tgg | 14.7091383 | 51.61055238 |
| | 1 | ctctgatgaacaagaccctc (SEQ ID NO: 186) | agg | 14.5402588 | 50.15205354 |
| | 1 | aaatcactcgcccttctcag (SEQ ID NO: 187) | agg | 14.2544365 | 65.66875202 |
| | −1 | gtgtcttgggcctctgagaa (SEQ ID NO: 188) | ggg | 13.9295958 | 49.88141773 |
| | 1 | tgatgaacaagaccctcagg (SEQ ID NO: 189) | agg | 13.7606138 | 68.32294117 |
| | −1 | ggtgtcttgggcctctgaga (SEQ ID NO: 190) | agg | 13.7195437 | 37.36705244 |
| 3′ UTR | −1 | gtctaagtgctgcgttaaga (SEQ ID NO: 191) | ggg | 84.6859457 | 48.63417996 |
| | −1 | tgtctaagtgctgcgttaag (SEQ ID NO: 192) | agg | 81.7777316 | 45.91914405 |
| | 1 | gagggagcctcttgtatgtt (SEQ ID NO: 193) | tgg | 76.8782493 | 35.37362392 |
| | 1 | gcccacctctccaacctaac (SEQ ID NO: 194) | tgg | 76.2988801 | 39.87036309 |
| | −1 | attgcagcctcaagtagact (SEQ ID NO: 195) | agg | 72.4829698 | 57.0478991 |
| | −1 | gactaggaagtaagccagtt (SEQ ID NO: 196) | agg | 72.2442137 | 46.00607944 |
| | −1 | aggaagtaagccagttaggt (SEQ ID NO: 197) | tgg | 71.9042489 | 41.7340746 |
| | 1 | gtaagccagttaggttggag (SEQ ID NO: 198) | agg | 71.54855 | 55.56736427 |
| | −1 | agactgaaagctagtctgag (SEQ ID NO: 199) | ggg | 69.6237996 | 65.42780553 |
| | 1 | cttacttcctagtctacttg (SEQ ID NO: 200) | agg | 67.8417065 | 55.35650233 |
| | 1 | gaagactgaaagctagtctg (SEQ ID NO: 201) | agg | 66.7107553 | 60.44061624 |
| | −1 | ctccctagaagacgccctca (SEQ ID NO: 202) | agg | 66.5546099 | 51.7559014 |
| | −1 | ctgaaagctagtctgagggg (SEQ ID NO: 203) | agg | 61.702876 | 67.32684796 |
| | 1 | ttgaggctgcaatcacactg (SEQ ID NO: 204) | agg | 61.31892 | 74.74770129 |
| | −1 | aagactgaaagctagtctga (SEQ ID NO: 205) | ggg | 60.6602522 | 54.56675355 |
| | −1 | gccagttaggttggagaggt (SEQ ID NO: 206) | ggg | 59.3258049 | 46.75048202 |
| | 1 | aggccttgagggcgtcttct (SEQ ID NO: 207) | agg | 58.5006215 | 28.99177389 |
| | −1 | tgggcaggggtcaagtgaaa (SEQ ID NO: 208) | tgg | 56.9295509 | 35.45301848 |
| | 1 | ggccttgagggcgtcttcta (SEQ ID NO: 209) | ggg | 56.5940243 | 41.23536534 |
| | 1 | cacaattccaaacatacaag (SEQ ID NO: 210) | agg | 55.9615864 | 61.51772376 |
| | −1 | ggggaggtggaacagcatga (SEQ ID NO: 211) | ggg | 54.6131044 | 69.50227076 |
| | −1 | aggggaggtggaacagcatg (SEQ ID NO: 212) | agg | 51.8017596 | 53.59962361 |
| | 1 | agccagttaggttggagagg (SEQ ID NO: 213) | tgg | 49.6070841 | 51.45946584 |
| | 1 | aaagctagtctgaggggagg (SEQ ID NO: 214) | tgg | 48.5212124 | 48.81089317 |
| | −1 | aggtggaacagcatgaggga (SEQ ID NO: 215) | agg | 47.5710532 | 63.15679382 |
| | −1 | tcaagtgaaatggagaactg (SEQ ID NO: 216) | tgg | 45.6822743 | 66.22020553 |
| | −1 | caagtgaaatggagaactgt (SEQ ID NO: 217) | ggg | 42.2396466 | 58.27515972 |
| | 1 | gttaggttggagaggtgggc (SEQ ID NO: 218) | agg | 42.1263881 | 37.29893803 |
| | 1 | tggaacagcatgagggaagg (SEQ ID NO: 219) | tgg | 41.6545549 | 56.47796615 |
| | 1 | acgccctcaaggcctgactg (SEQ ID NO: 220) | tgg | 35.5835131 | 65.38459652 |
| | −1 | gggcagacatgtttatttga (SEQ ID NO: 221) | agg | 33.9230448 | 36.96875759 |
| | 1 | taggttggagaggtgggcag (SEQ ID NO: 222) | ggg | 33.4801222 | 57.42383612 |
| | 1 | agacatgtttatttgaagga (SEQ ID NO: 223) | tgg | 30.5506105 | 52.39159675 |
| | −1 | gagtgtgttttttctccagc (SEQ ID NO: 224) | agg | 29.2922982 | 50.35103693 |
| | −1 | ttaggttggagaggtgggca (SEQ ID NO: 225) | ggg | 28.4425748 | 47.83669021 |
| | 1 | gacatttgtaagcaagtgag (SEQ ID NO: 226) | agg | 26.9419189 | 59.5267819 |
| | −1 | acattcagatttgtggtgtg (SEQ ID NO: 227) | agg | 26.4256821 | 59.99750155 |
| | −1 | gagaggcacattcagatttg (SEQ ID NO: 228) | tgg | 23.7410105 | 49.60496574 |
| | 1 | aaggcatgagtctgcatctt (SEQ ID NO: 229) | agg | 22.5261758 | 31.64102817 |
| | 1 | gttttttctccagcaggcag (SEQ ID NO: 230) | tgg | 21.269244 | 46.35960697 |
| | −1 | gagaactgtgggctaagcaa (SEQ ID NO: 231) | agg | 20.1230219 | 57.13606536 |
| | 1 | aggcatgagtctgcatctta (SEQ ID NO: 232) | ggg | 18.8279913 | 45.29561932 |
| | −1 | ggcctgactgtggtgctcat (SEQ ID NO: 233) | ggg | 18.0906072 | 46.09773639 |
| | −1 | agattccagctgctggtaca (SEQ ID NO: 234) | tgg | 18.0603432 | 49.56795823 |
| | 1 | gtacatgggagctggcaacc (SEQ ID NO: 235) | cgg | 17.8858383 | 63.6982707 |
| | 1 | cacttgcttacaaatgtcta (SEQ ID NO: 236) | agg | 16.9204605 | 48.62217882 |
| | −1 | agctgctggtacatgggagc (SEQ ID NO: 237) | tgg | 16.819902 | 45.14789672 |
| | 1 | agcaccacagtcaggccttg (SEQ ID NO: 238) | agg | 16.725956 | 60.15631648 |
| | 1 | ctaaggtccccactgcctgc (SEQ ID NO: 239) | tgg | 16.4850162 | 46.35050595 |
| | −1 | ttttttctccagcaggcagt (SEQ ID NO: 240) | ggg | 16.3504127 | 43.18290306 |
| | 1 | aggcctgactgtggtgctca (SEQ ID NO: 241) | tgg | 15.9475718 | 39.75667041 |
| | 1 | agctcccatgtaccagcagc (SEQ ID NO: 242) | tgg | 15.7399196 | 43.24124596 |
| | −1 | ggcaacccggttttgagaca (SEQ ID NO: 243) | ggg | 15.2164422 | 53.50908449 |
| | 1 | tttttctccagcaggcagtg (SEQ ID NO: 244) | ggg | 15.0293785 | 66.12065141 |
| | −1 | tggcaacccggttttgagac (SEQ ID NO: 245) | agg | 14.9169303 | 43.26076066 |
| | 1 | caacagccctgtctcaaaac (SEQ ID NO: 246) | cgg | 14.6632947 | 41.10299921 |
| | 1 | gcaccacagtcaggccttga (SEQ ID NO: 247) | ggg | 14.4012919 | 54.47726453 |
| | 1 | aacagccctgtctcaaaacc (SEQ ID NO: 248) | ggg | 14.0824143 | 61.19510127 |
| | −1 | gattccagctgctggtacat (SEQ ID NO: 249) | ggg | 12.8461481 | 42.84337761 |
| | -1 | tgccttcagattccagctgc (SEQ ID NO: 250) | tgg | 12.6088296 | 43.1813332 |
| | 1 | taccagcagctggaatctga (SEQ ID NO: 251) | agg | 11.2821164 | 58.57564638 |

Cpfi (Cas12)

| | | | | Specificity | Efficiency |
|---|---|---|---|---|---|
| | | | | Score (Off | Score (On |
| Region targeted | Strand | Sequence | PAM | target score) | target score) |
| KIR2DL2 promotcr | −1 | taccttgcatgaggcccagtg (SEQ ID NO: 252) | tttc | 12.4068985 | |
| | −1 | catgttgaccacatcatggga (SEQ ID NO: 253) | ttta | 13.26842 | |
| | −1 | gcgccctgcccatgcagttta (SEQ ID NO: 254) | tttg | 13.282476 | |
| Signal peptide + | 1 | cagggttcttcttgctgcagg (SEQ ID NO: 255) | tttc | 17.9146425 | |
| Exon 2 | 1 | tttccagggttcttcttgctg (SEQ ID NO: 256) | tttc | 16.1254393 | |
| Exon 3 | −1 | gagaccccatcatggtgctct (SEQ ID NO: 257) | tttg | 96.1936289 | |
| | 1 | aggacactttgcacctcattg (SEQ ID NO: 258) | ttta | 85.2555973 | |
| | 1 | cacctcattggagagcaccat (SEQ ID NO: 259) | tttg | 38.7284003 | |
| | −1 | accaggcgacctgggtgggcc (SEQ ID NO: 260) | tttc | 24.7183139 | |
| | 1 | agcacttccttctgcacagag (SEQ ID NO: 261) | tttg | 17.5260664 | |
| Exon 4 | −1 | accactcgtatggagagtcac (SEQ ID NO: 262) | tttg | 39.5758627 | |
| | 1 | cgtgactctccatacgagtgg (SEQ ID NO: 263) | tttc | 39.261553 | |
| | 1 | ctctgggccctgccacccacg (SEQ ID NO: 264) | tttc | 16.4322184 | |
| Exon 5 | −1 | agagcttggttcagtgggtga (SEQ ID NO: 265) | tttt | 38.9033407 | |
| | −1 | gagcttggttcagtgggtgaa (SEQ ID NO: 266) | ttta | 22.0523188 | |
| Exon 6 | −1 | gttggagcaccagcgatgaag (SEQ ID NO: 267) | tttt | 33.1804647 | |
| | −1 | ttggagcaccagcgatgaagg (SEQ ID NO: 268) | tttg | 33.1493374 | |
| | −1 | tgttggagcaccagcgatgaa (SEQ ID NO: 269) | tttt | 33.0688783 | |
| | −1 | tttgttggagcaccagcgatg (SEQ ID NO: 270) | tttt | 33.0291427 | |
| | −1 | ttgttggagcaccagcgatga (SEQ ID NO: 271) | tttt | 28.3779427 | |
| | 1 | tccttcatcgctggtgctcca (SEQ ID NO: 272) | tttc | 22.0003974 | |
| Exon 8 | −1 | gaagttccgtgtacacgatga (SEQ ID NO: 273) | tttg | 33.0685405 | |
| | −1 | gatctggactcagcatttgga (SEQ ID NO: 274) | tttg | 32.3627985 | |
| | −1 | tctgtgtgaaaacgcagtgat (SEQ ID NO: 275) | tttc | 15.1976376 | |
| | −1 | ctctgtgtgaaaacgcagtga (SEQ ID NO: 276) | tttt | 15.1682898 | |
| | −1 | tctctgtgtgaaaacgcagtg (SEQ ID NO: 277) | tttt | 15.1501405 | |
| | 1 | acacagagaaaaatcactcgc (SEQ ID NO: 278) | tttc | 14.9951438 | |
| | 1 | cacacagagaaaaatcactcg (SEQ ID NO: 279) | tttt | 14.7463876 | |
| 3′ UTR | 1 | acttgacccctgcccacctct (SEQ ID NO: 280) | tttc | 94.5211894 | |
| | −1 | ctccagcaggcagtggggacc (SEQ ID NO: 281) | tttt | 92.6667631 | |
| | −1 | gaattgtgagttcctcagtgt (SEQ ID NO: 282) | tttg | 91.0259628 | |
| | −1 | actgctgacagaagactgaaa (SEQ ID NO: 283) | tttt | 88.8907291 | |
| | −1 | tccagcaggcagtggggacct (SEQ ID NO: 284) | tttc | 86.4077082 | |
| | 1 | agtcttctgtcagcagtaaaa (SEQ ID NO: 285) | tttc | 86.2360603 | |
| | −1 | ctgctgacagaagactgaaag (SEQ ID NO: 286) | ttta | 85.6577309 | |
| | −1 | taagcaagtgagaggcacatt (SEQ ID NO: 287) | tttg | 61.6642353 | |
| | −1 | aaggatggaaaactacattga (SEQ ID NO: 288) | tttg | 59.4762322 | |
| | −1 | tttgaaggatggaaaactaca (SEQ ID NO: 289) | ttta | 59.4515509 | |
| | 1 | ccatccttcaaataaacatgt (SEQ ID NO: 290) | tttt | 57.7176326 | |
| | 1 | catccttcaaataaacatgtc (SEQ ID NO: 291) | tttc | 55.7785529 | |
| | 1 | ttaaaataacttcaatgtagt (SEQ ID NO: 292) | tttt | 55.0105568 | |
| | 1 | aaataacttcaatgtagtttt (SEQ ID NO: 293) | ttta | 53.5669934 | |
| | 1 | taaaataacttcaatgtagtt (SEQ ID NO: 294) | tttt | 51.3980311 | |
| | 1 | aaaataacttcaatgtagttt (SEQ ID NO: 295) | tttt | 51.0928346 | |
| | −1 | aaaaaatatataagttttact (SEQ ID NO: 296) | tttt | 37.2680576 | |
| | −1 | ttctccagcaggcagtgggga (SEQ ID NO: 297) | tttt | 32.1407638 | |
| | −1 | aaaaatatataagttttactg (SEQ ID NO: 298) | ttta | 31.0317963 | |
| | −1 | tggtgtgaggaagagcgatgc (SEQ ID NO: 299) | tttg | 28.1189674 | |
| | 1 | cttagcccacagttctccatt (SEQ ID NO: 300) | tttg | 27.8217365 | |
| | −1 | tctccagcaggcagtggggac (SEQ ID NO: 301) | tttt | 16.3318587 | |
| | −1 | gagacagggctgttgtctccc (SEQ ID NO: 302) | tttt | 15.2323502 | |
| | −1 | agacagggctgttgtctccct (SEQ ID NO: 303) | tttg | 13.2493833 | |

TABLE 2
KIR2DL2 guide RNA (gRNA*) and CRISPR RNA (crRNA*) selected for KIR2DL2 knockout
Cas9

| | | | | On | Off |
|---|---|---|---|---|---|
| gRNA | PAM | Sequence (5′-3′) | Target | target core | target score |
| gRNAe1 | TGG | AGACAGCACCATGTCGCTCA (SEQ ID NO: 28) | Exon 1 | 61.7 | 65.8 |
| gRNAe3_1 | GGG | ATGATGCAAGACCTTGCAGG (SEQ ID NO: 304) | Exon 3 | 41.9 | 79.6 |
| gRNAe3_2 | AGG | GCAAGGTCTTGCATCATGGG (SEQ ID NO: 305) | Exon 3 | 47.8 | 72.5 |
| gRNAe3_3 | TGG | CCCTGCAAGGTCTTGCATCA (SEQ ID NO: 44) | Exon 3 | 50.8 | 72.4 |
| gRNAe3_4 | GGG | CCATGATGCAAGACCTTGCA (SEQ ID NO: 45) | Exon 3 | 60.9 | 68.7 |
| gRNAe3_5 | TGG | TCTGATTTCACCAGGCGACC (SEQ ID NO: 50) | Exon 3 | 62 | 74 |
| gRNAe3_6 | GGG | CTGATTTCACCAGGCGACCT (SEQ ID NO: 55) | Exon 3 | 62 | 74 |
| gRNAe4_1 | TGG | CTGCAGAGAACCTACATTCA (SEQ ID NO: 87) | Exon 4 | 41.8 | 58.1 |
| gRNAe4_2 | AGG | AGGGGGAGGCCCATGAATGT (SEQ ID NO: 88) | Exon 4 | 54.9 | 57.6 |
| gRNAe4_3s | GGG | ATGAATGTAGGTTCTCTGCA (SEQ ID NO: 91) | Exon 4 | 63.7 | 43 |
| gRNAe4_3as | GGG | TGCAGAGAACCTACATTCAT (SEQ ID NO: 89) | Exon 4 | 47.2 | 53.8 |
| gRNAe4_4 | TGG | TCCGTGACTCTCCATACGAG (SEQ ID NO: 92) | Exon 4 | 69 | 79 |
| gRNAe4_5 | CGG | ACCACTCGTATGGAGAGTCA (SEQ ID NO: 97 | Exon 4 | 67 | 61 |
| gRNAe4_6 | GGG | GCATCTGTAGGTTCCTCCGT (SEQ ID NO: 103) | Exon 4 | 65 | 57 |
| gRNAe4_7 | TGG | CTCGAGTTTGACCACTCGTA (SEQ ID NO: 96) | Exon 4 | 54 | 65 |
| gRNAe4_8 | GGG | TGGAATGTTCCGTTGACCTT (SEQ ID NO: 101) | Exon 4 | 59 | 52 |
| gRNAe4_9 | TGG | CTGGAATGTTCCGTTGACCT (SEQ ID NO: 100) | Exon 4 | 56 | 52 |
| gRNAe4_10 | TGG | ATTCCAGGCCGACTTTCCTC (SEQ ID NO: 112) | Exon 4 | 55 | 46 |
| gRNAe4_6 | TGG | CGACACCTGCACATTCTGAT (SEQ ID NO: 152) | Exon 6 | 59 | 57 |
| gRNAe7_1 | AGG | GTAATGGACCAAGAGTCTGC (SEQ ID NO: 171) | Exon 7 | 97 | 67 |
| gRNAe7_2 | GGG | TAATGGACCAAGAGTCTGCA (SEQ ID NO: 168) | Exon 7 | 79 | 43 |
| gRNAe8_1 | CGG | AACAGATATCATCGTGTACA (SEQ ID NO: 173) | Exon 8 | 62.7 | 31.2 |
| gRNAe8_2 | GGG | GTACACGATGATATCTGTTG (SEQ ID NO: 174) | Exon 8 | 62 | 31 |
| gRNAe8_3 | TGG | GTGTACACGATGATATCTGT (SEQ ID NO: 175) | Exon 8 | 76 | 59 |
| gRNAe8_4 | GGG | TACACGATGATATCTGTTGG (SEQ ID NO: 177) | Exon 8 | 63 | 54 |

Cpf1

| | | | | On | Off |
|---|---|---|---|---|---|
| gRNA | PAM | Sequence (5′-3′) | Target | target core | target score |
| crRNAe2 | TTTG | gagaaggactcacCCTCATGTGG (SEQ ID NO: 306) | Exon 2 | 87.6 | |
| crRNAe3 1 | TTTA | ATGATGCAAGACCTTGCAGG (SEQ ID NO: 304) | Exon 3 | | 96.9 |
| crRNAe3_2 | TTTG | GCAAGGTCTTGCATCATGGG (SEQ ID NO: 305) | Exon 3 | | 87.7 |
| crRNA 4-12 | TTTG | GAGACCCCATCATGGTGCTC (SEQ ID NO: 307) | Exon 3 | | 96.2 |
| crRNA 5en12 | TTTC | AAAGCCAACTTCTCCATCGG (SEQ ID NO: 308) | Exon 3 | | |
| crRNA e8 | TTTC | TCTGTGTGAAAACGCAGTGAT (SEQ ID NO: 275) | Exon 8 | 50.7 | |
| crRNA e8_2 | TTTG | GAAGTTCCGTGTACACGATGATA (SEQ ID NO: 309) | Exon 8 | 87.6 | |
| crRNA e8_3 | TTTC | ACACAGAGAAAAATCACTCGCCC (SEQ ID NO: 310) | Exon 8 | 684 | |
| crRNA e8_4 | TTTG | GATCTGGACTCAGCATTTGGAAG (SEQ ID NO: 311) | Exon 8 | 53.6 | |

Also disclosed are methods of disrupting KIR2DL2 expression in T cells ex vivo while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous KIR2DL2 protein.

In other embodiments, the subject is treated with a KIR2DL2 inhibitor prior to, during, or after treatment with adoptive cell immunotherapy. For example, the KIR2DL2 inhibitor can be a blocking antibody that binds KIR2DL2 Ig domains and blocks its binding to an HLA-C1. In some embodiments, the blocking antibody binds an HLA-C1 and blocks its binding to KIR2DL2 without blocking its binding to TCRs. Soluble receptors, such as KIR2DL2 fragments or HLA-C fragments that block this interaction are also contemplated for use in the disclosed methods. The structure or human KIR2DL2 and its binding to HLA-Cw3 is described in Boyington, J C, et al. Nature 2000 405:537-43, which is incorporated by reference for the teaching of these binding sites.

### Chimeric Antigen Receptors (CAR)

In particular embodiments, the immune effector cells with ablated KIR2DL2 are engineered to express a chimeric antigen receptor (CAR) polypeptide. CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41 BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ξ domain. TanCAR T cell activation is achieved only when target cells co-express both targets.

A dual CAR T cell expresses two separate CARs with different ligand binding targets; one CAR includes only the CD3ξ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ξ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ξ) (TCR zeta, GenBank accno. BAG36664.1). T-cell surface glycoprotein CD3 zeta (CD3ξ) chain, also known as T-cell receptor T3 zeta chain or CD247 (Cluster of Differentiation 247), is a protein that in humans is encoded by the CD247 gene.

First-generation CARs typically had the intracellular domain from the CD3ξ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ξ signaling domain by itself or combined with any other desired cytoplasmic domain(s) useful in the context of the CAR of the invention. For example, the cytoplasmic domain of the CAR can comprise a CD34 chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gpIOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, FIt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCASI, SDCCAG1 6, TA-90Mac-2 binding proteincyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors. Also disclosed are oligonucleotides for use in inserting the chimeric receptors into the genome of a T cell at a site that will disrupt Sirt2 expression or activity.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Immune Effector Cells

Also disclosed are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as FicoII™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8+T lymphocytes. In some embodiments, the T cells comprise γδ T cells, which possess a distinct T-cell receptor (TCR) having one γchain and one δ chain instead of a and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺T lymphocytes, NK cells launch cytotoxicity against tumor cells without the requirement for prior sensitization, and can also eradicate MHC-1-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### Therapeutic Methods

Immune effector cells expressing the disclosed chimeric receptors can elicit an anti-tumor immune response against cancer cells. The anti-tumor immune response elicited by the disclosed chimeric cells may be an active or a passive immune response. In addition, the immune response may be part of an adoptive immunotherapy approach in which chimeric cells induce an immune response specific to the target antigen.

Adoptive transfer of immune effector cells expressing chimeric receptors is a promising anti-cancer therapeutic. Following the collection of a patient's immune effector cells, the cells may be genetically engineered to express the disclosed chimeric receptors while ablating Sirt2 according to the disclosed methods, then infused back into the patient.

The disclosed chimeric effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁸ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed chimeric cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed chimeric cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed chimeric cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed chimeric cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with chimeric cells for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed chimeric cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Killer cell immunoglobulin-like receptors (KIRs) are transmembrane glycoproteins expressed by natural killer cells and subsets of T cells. They are classified based on the number of extracellular immunoglobulin (Ig) domains, and on the length of their intracellular domain.

KIR2DL2, for example, contains 2 extracellular Ig domains, and a long intracellular tail. KIR2DL2 binds HLA molecules of the C1 group (Cw1, Cw3, Cw7 and Cw8) resulting in inhibition of Natural Killer (NK) cells, through a mechanism that involves the phosphatase SHP1. The role of KIR2DL2 as an inhibitory receptor in T cells is less characterized, but it has been reported that it can inhibit TCR signaling by interfering with protein-protein interactions in the immune synapse.

It was observed that adoptively transferred CAR-T cells upregulate KIR2DL2 in vivo, in mouse models of pancreatic cancer. This is in line with a previous report showing increased expression in vivo in TCR-transgenic T cells administered to melanoma patients.

Finally, there was spontaneous expression of KIR2DL2 in gamma/delta T cells from melanoma patients. Binding of HLA-C1 molecules (expressed by target cells) through KIR2DL2 (expressed by T cells) can cause inhibition of CAR-mediated tumor lysis. Moreover, if tumor recognition is mediated by an HLA-C1-restricted TCR, KIR2DL2 may directly bind the TCR/peptide-HLA complex, causing its inhibition.

Upregulation of KIR2DL2 in patients who received TCR-transgenic T cells

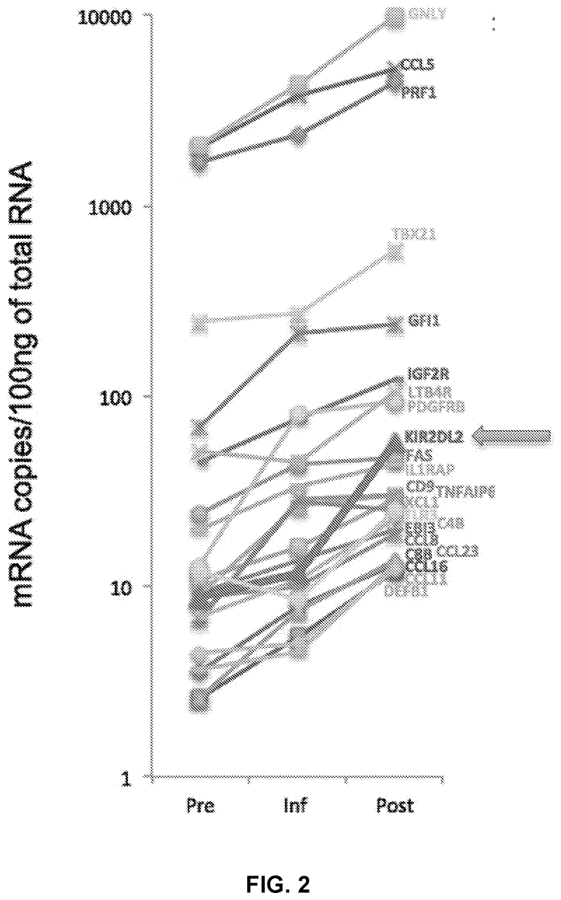

FIG. 2 shows Nanostring analysis of gene expression in peripheral blood T cells, collected from melanoma and synovial cell sarcoma patients. Pre-infusion samples (Pre) are unmodified T cells; Infusion samples (Inf) are genetically modified T cells as they were administered to patients; Post-infusion (Post) cell are peripheral blood T lymphocytes collected 1 month post adoptive transfer.

KIR2DL2 RNA expression in PSCA CAR-T cells, before and after adoptive transfer into NSG mice bearing HPAC Tumors.

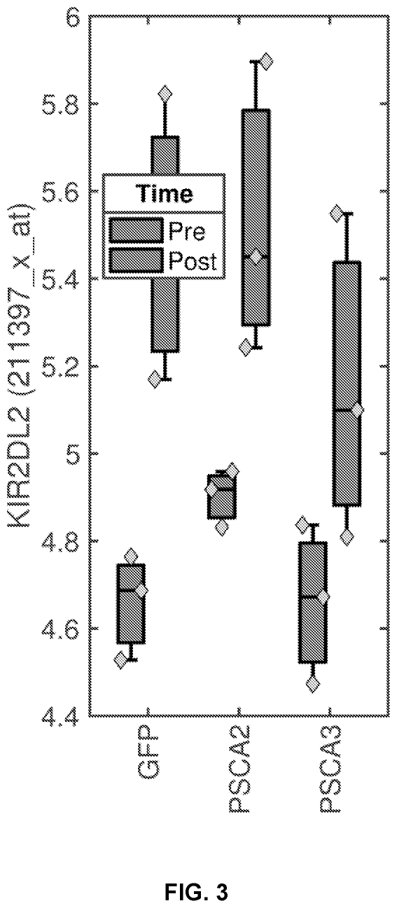

FIG. 3 shows human peripheral blood T cells were genetically modified to express a second-generation CAR (PSCA2), a third-generation CAR (PSCA3), or GFP as a negative control. Following 14 days of expansion ex vivo, the T cells were administered to immunodeficient mice bearing subcutaneous xenografts of HPAC pancreatic cancer cells. Thirty days after infusion, splenocytes were collected. The gene expression of these post-infusion T cells (Post) was compared to that of the infusion product (Pre). The box plot shows the relative expression values for each condition (in triplicates).

Panc02.03 pancreatic cancer cells express receptors for KIR2DL2.

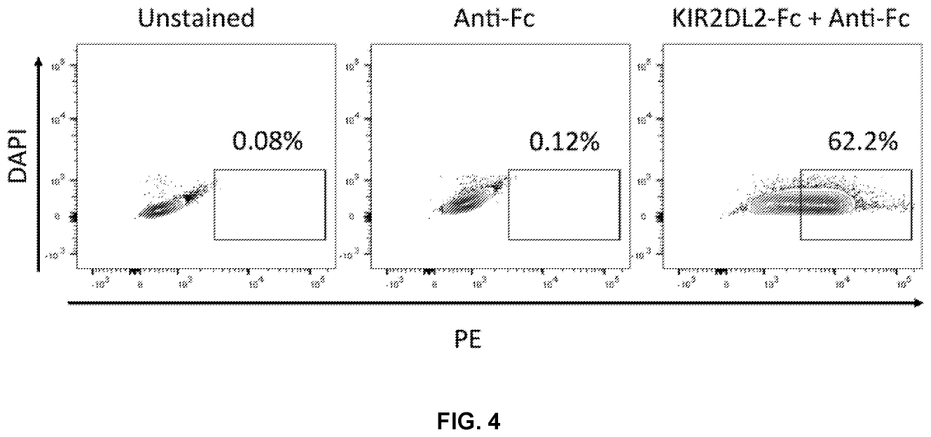

FIG. 4 shows panc02.03 pancreatic cancer cells incubated with a fusion protein encompassing the extracellular domain of KIR2DL2 linked to the Fc portion of an antibody (KIR2DL2-Fc). Then, a secondary staining with a PE-conjugated anti-Fc reagent (Anti-Fc) was applied, showing binding in over 60% of Panc02.03 cells. Negative controls of unstained Panc02.03 cells, or cells with only secondary staining, were used to establish the gating strategy.

### Example 2

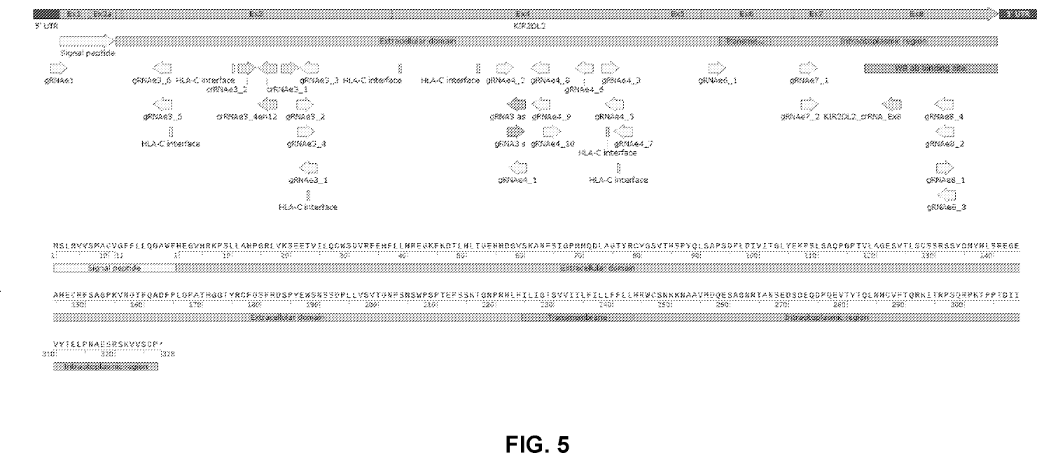

FIG. 5: Schematic representation of the KIR2DL2 gene and its protein sequence. A) KIR2DL2 coding sequence (hg38-NM 014219.2), including exons and both the 5′ and 3′ UTR, is depicted. Guide RNAs (gRNAs) and CRISPR RNAs (crRNAs) are represented by green and orange arrows, respectively. B) KIR2DL2 protein sequence consensus (NP_055034) with the different protein regions. Maps and features were created and represented with SnapGene software (Insightful Science).

TABLE 3
List of KIR2DL2 sequenced alleles and characterized protein
sequences. All known KIR2DL2 gene coding and protein sequences
were retrieved from the EMBL-EBI database and summarized.

| Allele Name | Local Names | Cells Sequenced | Accession No. |
|---|---|---|---|
| 2DL2*0010101 | p58 cl-43 | NK-CM, | AY320039, |
| | | RDP9, | EU791544, |
| | | WT47, | U24075, |
| 2DL2*0010102 | FH05_B | FH05, | GU182339, |
| 2DL2*0010103 | G085_BA1 | G085, | GU182349, |
| 2DL2*0010104 | RSH_BA2 | RSH, | GU182357, |
| 2DL2*0010105 | 2DL2_FH13_BA2_hap | FH13, | GU182345, |
| 2DL2*0010106 | FH15_B_hap | FH15, | GU182347, |
| 2DL2*0010107 | 2DL2_G248_BA2hap | G248, | GU182351, |
| 2DL2*00102 | GM17140 | GM17140, | EU933931, |
| 2DL2*00103 | KIR2DL2 | BTI000024, | KP260924, |
| 2DL2*002 | NKAT6 | UNK2, | L76669, |
| 2DL2*0030101 | NKAT6, | MU, | AF285434, |
| | 2DL2v2 | RDP11, | AL133414, |
| | | RPCI-1, | EU791545, |
| 2DL2*0030102 | 2DL2 − CU467816 | PGF, | CU467816, |
| 2DL2*0030103 | MC1B − CU464060 2DL2 | MC1B, | CU464060, |
| 2DL2*0030104 | FH06_BA1 | FH06, | GU182341, |
| 2DL2*0030105 | GRC212_BA1 | GRC-212, | GU182353, |
| 2DL2*0030106 | LUCE_Bdel | LUCE, | GU182355, |
| 2DL2*0030107 | T7526_Bdel | T7526, | GU182359, |
| 2DL2*0030108 | 2DL2_00301_non_coding_variant_16 | D05392, | LR593893, |
| 2DL2*00302 | CHSCT13KL2 | 15563819, | GQ921919, |
| 2DL2*00303 | ML1643 | ML1643, | FJ188690, |
| 2DL2*00304 | ML1560 | ML1560, | FJ188691, |
| 2DL2*004 | 2DL2v1 | GM17167, | AF285433, |
| | | WC, | GU138985, |
| 2DL2*005 | 2DL2-094M | 094M, | AY366242, |
| | | RDP13, | EU791546, |
| 2DL2*00601 | GM17109 | GM17109, | EU933932, |
| | | ML2079, | FJ188688, |
| 2DL2*00602 | GM17115 | GHA-020, | EU933933, |
| | | GM17115, | FJ188689, |
| | | ML1346, | HM211186, |
| 2DL2*007 | GM17134 | GM17134, | EU933935, |
| 2DL2*008 | CHSCT03KL2 | 15558873, | GQ921915, |
| 2DL2*009 | CHSCT22KL2 | 16121885, | GQ921914, |
| 2DL2*010 | CHSCT27KL2 | 16216138, | GQ921918, |
| 2DL2*011 | KIR2DL2_004LIKE | GHA-160M, | HM211185, |
| 2DL2*012 | KIR2DL2sa052 | SA052, | JX523631, |
| 2DL2*013 | KIR2DL2 | 12019800, | KM017076, |
| 2DL2*014 | 2DL2*00301_118c_470g | LIFT29, | MH938274, |
| 2DL2*015 | 2DL2*00101_110c_246g_505c | LIFT50, | MH938273, |

TABLE 4
List of amino acid changes between KIR2DL2 protein
variants. All known KIR2DL2 protein sequences were
aligned using the SnapGene software and the amino
acid changes respect to consensus were summarized.

| Allele | Differences respect to consensus |
|---|---|
| 2DL2*0010101 | Pos 200 −> T to I |
| | Pos 312 −> T to A |
| 2DL2*0010102 | Pos 200 −> T to I |
| | Pos 312 −> T to A |
| 2DL2*0010103 | Pos 200 −> T to I |
| | Pos 312 −> T to A |
| 2DL2*0010104 | Pos 200 −> T to I |
| | Pos 312 −> T to A |
| 2DL2*0010105 | Pos 200 −> T to I |
| | Pos 312 −> T to A |
| 2DL2*0010106 | Pos 200 −> T to I |
| | Pos 312 −> T to A |
| 2DL2*0010107 | Pos 200 −> T to I |
| | Pos 312 −> T to A |
| 2DL2*00102 | Pos 200 −> T to I |
| | Pos 312 −> T to A |
| 2DL2*00103 | Pos 200 −> T to I |
| | Pos 312 −> T to A |
| 2DL2*002 | Pos 200 −> T to I |
| | Pos 268 −> S to R |
| | Pos 269 −> E to Q |
| | Pos 312 −> T to A |
| 2DL2*0030101 | |
| 2DL2*0030102 | |
| 2DL2*0030103 | |
| 2DL2*0030104 | |
| 2DL2*0030105 | |
| 2DL2*0030106 | |
| 2DL2*0030107 | |
| 2DL2*0030108 | |
| 2DL2*00302 | |
| 2DL2*00303 | |
| 2DL2*00304 | |
| 2DL2*004 | Pos 16 −> R to P |
| | Pos 35 −> E to Q |
| | Pos 41 −> R to T |
| | Pos 167 −> G to D |
| | Pos 216 −> K to E |
| | Pos 268 −> S to I |
| | Pos 296 −> R to H |
| | Pos 336 −> N to S |
| 2DL2*005 | Pos 200 −> T to I |
| 2DL2*00601 | Pos 16 −> R to P |
| 2DL2*00602 | Pos 16 −> R to P |
| 2DL2*007 | Pos 200 −> T to I |
| | Pos 232 −> V to A |
| | Pos 312 −> T to A |
| 2DL2*008 | Pos 200 −> T to I |
| | Pos 245 −> R to C |
| 2DL2*009 | Pos 148 −> C to R |
| 2DL2*010 | Pos 200 −> T to I |
| | Pos 245 −> R to C |
| | Pos 272 −> D to Y |
| 2DL2*011 | Pos 16 −> R to P |
| | Pos 167 −> G to D |
| | Pos 216 −> K to E |
| | Pos 268 −> S to I |
| | Pos 296 −> R to H |
| | Pos 336 −> N to S |
| 2DL2*012 | Pos 3 −> G to E |
| 2DL2*013 | Pos 248 −> S to C |
| | Pos 339 −> S to P |
| 2DL2*014 | Pos 19 −> K to Q |
| | Pos 136 −> M to R |
| 2DL2*015 | Pos 16 −> R to P |
| | Pos 148 −> C to R |
| | Pos 200 −> T to I |
| | Pos 312 −> T to A |

Table 5: List of gRNAs and crRNAs designed for KIR2DL2 knockout. A) List of guide RNAs (gRNAs) targeting different KIR2DL2 exons for Cas9 disruption of the coding sequence. B) List of CRISPR RNAs (crRNAs) targeting different KIR2DL2 exons for Cpf1 (Cas12) disruption of the coding sequence. On target and off target score were calculated by Benchling (Biology Software, 2021) using an algorithm previously described (Hsu, P. D., et al., DNA targeting specificity of RNA-guided Cas9 nucleases. Nat Biotechnol, 2013. 31(9): p. 827-32; Doench, J. G., et al., Optimized sgRNA design to maximize activity and minimize off-target effects of CRISPR-Cas9. Nat Biotechnol, 2016. 34(2): p. 184-191). The higher the on-target score, the higher the probability of the Cas protein to cut on the target site (higher specificity); the higher the off-target score, the lesser the probability of the Cas protein to cut outside of the target site (higher efficiency).

Table 5A
Cas9

| | | | | On | Off |
|---|---|---|---|---|---|
| gRNA | PAM | Sequence (5′-3′) | Target | target score | target score |
| gRNAe1 | TGG | AGACAGCACCATGTCGCTCA (SEQ ID NO: 28) | Exon 1 | 61.7 | 65.8 |
| gRNAe3_1 | GGG | ATGATGCAAGACCTTGCAGG (SEQ ID NO: 304) | Exon 3 | 41.9 | 79.6 |
| gRNAe3_2 | AGG | GCAAGGTCTTGCATCATGGG (SEQ ID NO: 305) | Exon 3 | 47.8 | 72.5 |
| gRNAe3_3 | TGG | CCCTGCAAGGTCTTGCATCA (SEQ ID NO: 44) | Exon 3 | 50.8 | 72.4 |
| gRNAe3_4 | GGG | CCATGATGCAAGACCTTGCA (SEQ ID NO: 45) | Exon 3 | 60.9 | 68.7 |
| gRNAe3_5 | TGG | TCTGATTTCACCAGGCGACC (SEQ ID NO: 50) | Exon 3 | 62 | 74 |
| gRNAe3_6 | GGG | CTGATTTCACCAGGCGACCT (SEQ ID NO: 55) | Exon 3 | 62 | 74 |
| gRNAe4_1 | TGG | CTGCAGAGAACCTACATTCA (SEQ ID NO: 87) | Exon 4 | 41.8 | 58.1 |
| gRNAe4_2 | AGG | AGGGGGAGGCCCATGAATGT (SEQ ID NO: 88) | Exon 4 | 54.9 | 57.6 |
| gRNAe4_3s | GGG | ATGAATGTAGGTTCTCTGCA (SEQ ID NO: 91) | Exon 4 | 63.7 | 43 |
| gRNAe4_3as | GGG | TGCAGAGAACCTACATTCAT (SEQ ID NO: 89) | Exon 4 | 47.2 | 53.8 |
| gRNAe4_4 | TGG | TCCGTGACTCTCCATACGAG (SEQ ID NO: 92) | Exon 4 | 69 | 79 |
| gRNAe4_5 | CGG | ACCACTCGTATGGAGAGTCA (SEQ ID NO: 97) | Exon 4 | 67 | 61 |
| gRNAe4_6 | GGG | GCATCTGTAGGTTCCTCCGT (SEQ ID NO: 103) | Exon 4 | 65 | 57 |
| gRNAe4_7 | TGG | CTCGAGTTTGACCACTCGTA (SEQ ID NO: 96) | Exon 4 | 54 | 65 |
| gRNAe4_8 | GGG | TGGAATGTTCCGTTGACCTT (SEQ ID NO: 101) | Exon 4 | 59 | 52 |
| gRNAe4_9 | TGG | CTGGAATGTTCCGTTGACCT (SEQ ID NO: 100) | Exon 4 | 56 | 52 |
| gRNAe4_10 | TGG | ATTCCAGGCCGACTTTCCTC (SEQ ID NO: 112) | Exon 4 | 55 | 46 |
| gRNAe6 | TGG | CGACACCTGCACATTCTGAT (SEQ ID NO: 152) | Exon 6 | 59 | 57 |
| gRNAe7_1 | AGG | GTAATGGACCAAGAGTCTGC (SEQ ID NO: 171) | Exon 7 | 97 | 67 |
| gRNAe7_2 | GGG | TAATGGACCAAGAGTCTGCA (SEQ ID NO: 168) | Exon 7 | 79 | 43 |
| gRNAe8_1 | CGG | AACAGATATCATCGTGTACA (SEQ ID NO: 173) | Exon 8 | 62.7 | 31.2 |
| gRNAe8_2 | GGG | GTACACGATGATATCTGTTG (SEQ ID NO: 174) | Exon 8 | 62 | 31 |
| gRNAe8_3 | TGG | GTGTACACGATGATATCTGT (SEQ ID NO: 175) | Exon 8 | 76 | 59 |
| gRNAe8_4 | GGG | TACACGATGATATCTGTTGG (SEQ ID NO: 177) | Exon 8 | 63 | 64 |

Table 5B
Cpf1 (Cas12)

| | | | | On | Off |
|---|---|---|---|---|---|
| gRNA | PAM | Sequence (5′-3′) | Target | target score | target score |
| crRNAc2 | TTTG | gagaaggactcacCCTCATGTGG (SEQ ID NO: 306) | Exon 2 | 87.6 | |
| crRNAe3_1 | TTTA | ATGATGCAAGACCTTGCAGG (SEQ ID NO: 304) | Exon 3 | | 96.9 |
| crRNAe3_2 | TTTG | GCAAGGTCTTGCATCATGGG (SEQ ID NO: 305) | Exon 3 | | 87.7 |
| crRNA 4-12 | TTTG | GAGACCCCATCATGGTGCTC (SEQ ID NO: 307) | Exon 3 | | 96.2 |
| crRNA 5en12 | TTTC | AAAGCCAACTTCTCCATCGG (SEQ ID NO: 308) | Exon 3 | | |
| crRNA e8 | TTTC | TCTGTGTGAAAACGCAGTGAT (SEQ ID NO: 275) | Exon 8 | 50.7 | |
| crRNA e8_2 | TTTG | GAAGTTCCGTGTACACGATGATA (SEQ ID NO: 309) | Exon 8 | 87.6 | |
| crRNA e8_3 | TTTC | ACACAGAGAAAAATCACTCGCCC (SEQ ID NO: 310) | Exon 8 | 684 | |
| crRNA e8 4 | TTTG | GATCTGGACTCAGCATTTGGAAG (SEQ ID NO: 311) | Exon 8 | 53.6 | |

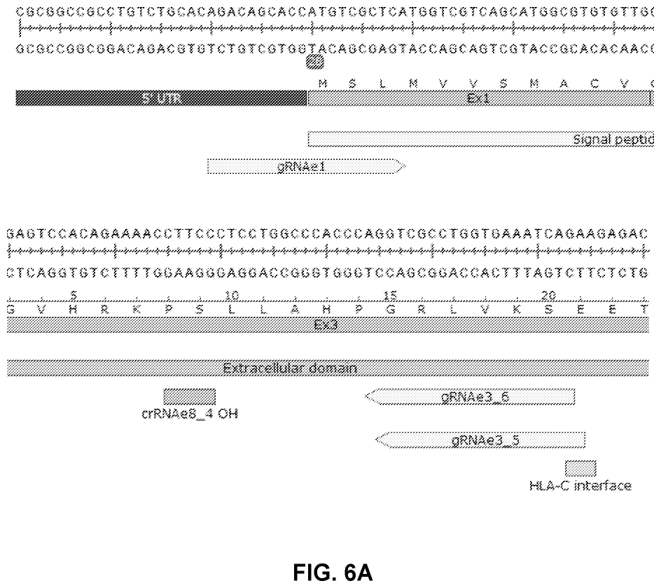

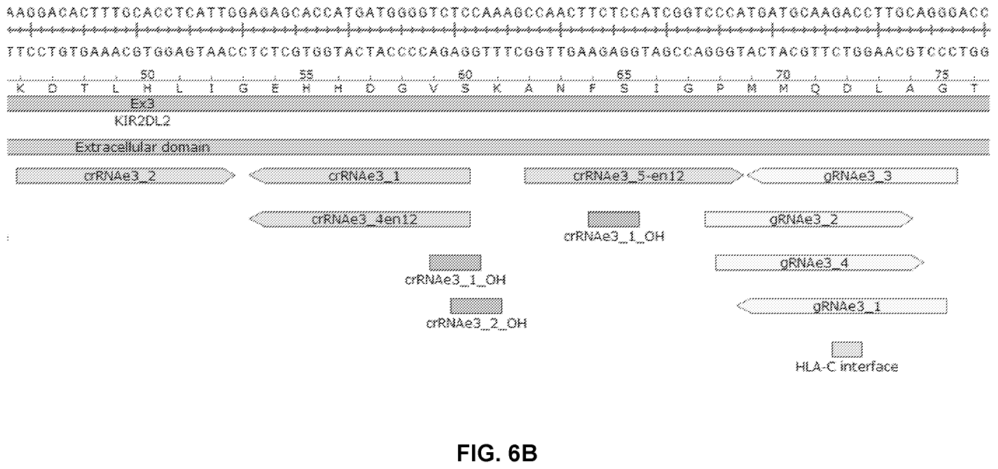

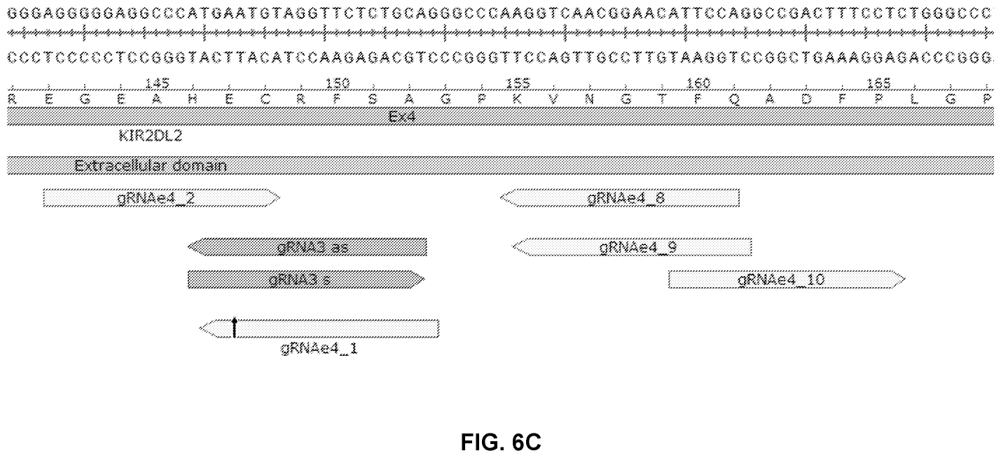

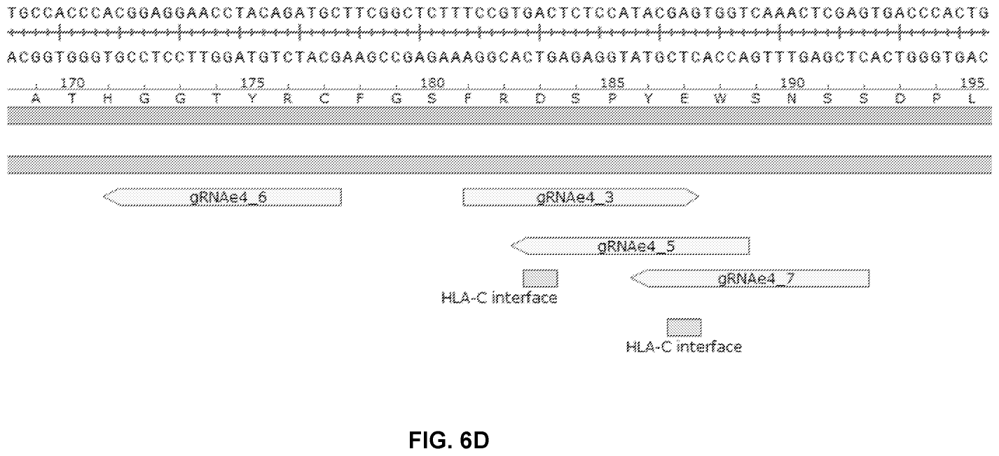

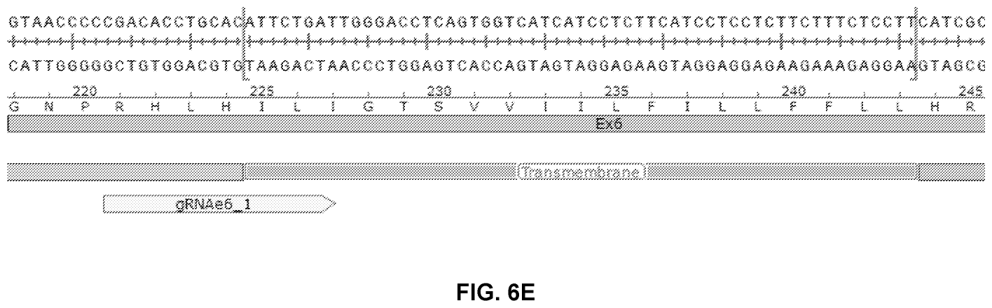

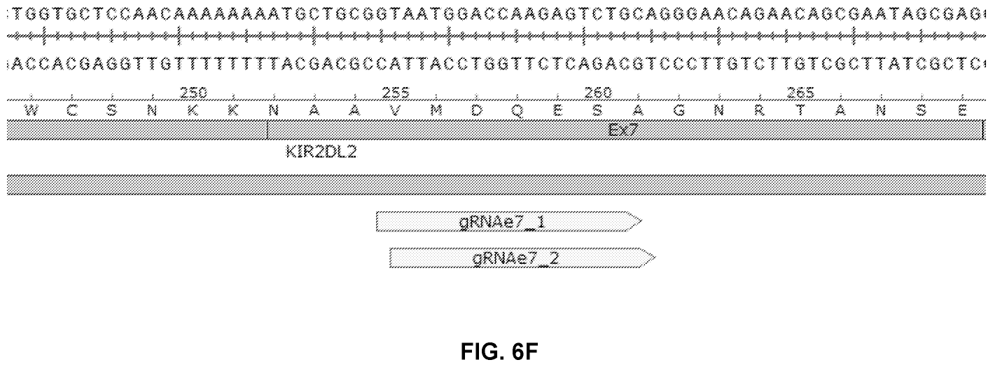

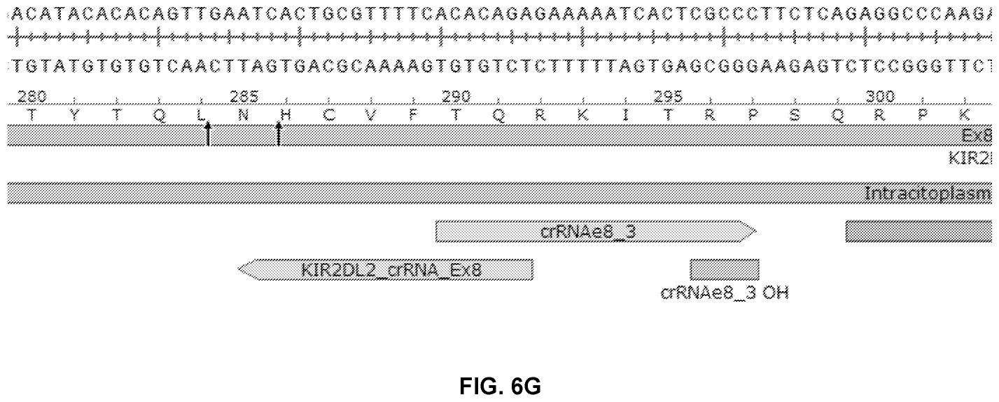

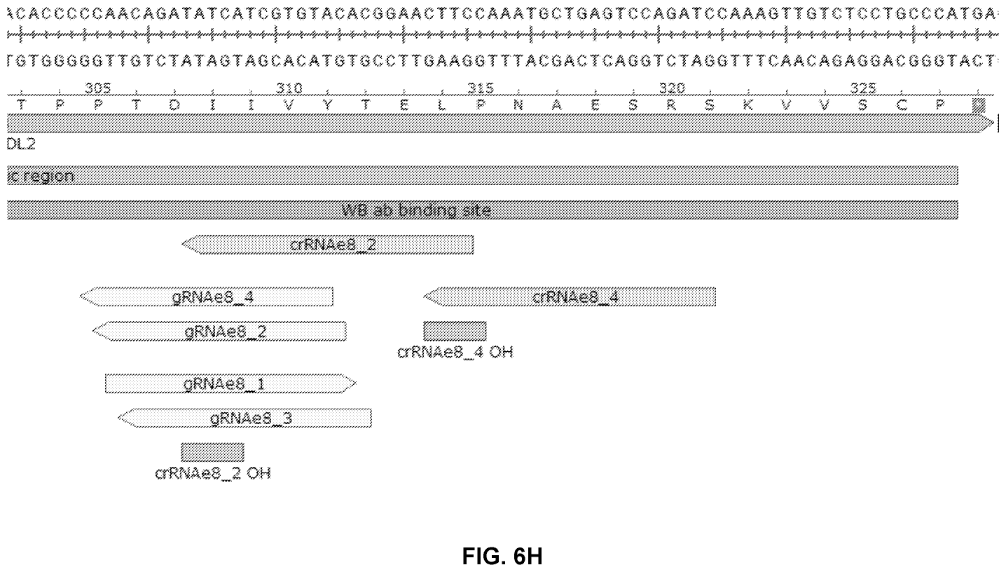

FIG. 6A-6H. Alignment of all designed gRNAs and crRNAs within the KIR2DL2 coding sequence. In green, gRNAs (Cas9) are indicated and aligned; in orange, crRNAs (Cas12) are indicated and aligned.

Table 6A and 6B. KIR2DL2 deletion: experimental design.—2×105 human T cells known to express KIR2DL2 were nucleofected with both gRNAs or crRNAs targeting the housekeeping gene HPRT1 or different KIR2DL2 exons (see table below).—48-72 hours after transfection, DNA was extracted, and every targeted exon amplified to assess Cas9/Cas12 cut by a T7E1 cleavage assay.

Table 6A
Cas9

| | | | | On | Off |
|---|---|---|---|---|---|
| gRNA | PAM | Sequence (5′-3′) | Target | target score | target score |
| 1. gRNAe1 | TGG | AGACAGCACCATGTCGCTCA (SEQ ID NO: 28) | Exon 1 | 61.7 | 65.8 |
| 6. gRNAe3_1 | GGG | ATGATGCAAGACCTTGCAGG (SEQ ID NO: 304) | Exon 3 | 41.9 | 79.6 |
| 7. gRNAe3_2 | AGG | GCAAGGTCTTGCATCATGGG (SEQ ID NO: 305) | Exon 3 | 47.8 | 72.5 |
| 8. gRNAe3_3 | TGG | CCCTGCAAGGTCTTGCATCA (SEQ ID NO: 44) | Exon 3 | 50.8 | 72.4 |
| 9. gRNAe3_4 | GGG | CCATGATGCAAGACCTTGCA (SEQ ID NO: 45) | Exon 3 | 60.9 | 68.7 |
| 10. gRNAe4_1 | TGG | CTGCAGAGAACCTACATTCA (SEQ ID NO: 87) | Exon 4 | 41.8 | 58.1 |
| 11. gRNAe4_2 | AGG | AGGGGGAGGCCCATGAATGT (SEQ ID NO: 88) | Exon 4 | 54.9 | 57.6 |
| 12. gRNAe4_3s | GGG | ATGAATGTAGGTTCTCTGCA (SEQ ID NO: 91) | Exon 4 | 63.7 | 43 |
| 13. gRNAe4_3as | GGG | TGCAGAGAACCTACATTCAT (SEQ ID NO: 89) | Exon 4 | 47.2 | 53.8 |
| 14. gRNAe8_1 | CGG | AACAGATATCATCGTGTACA (SEQ ID NO: 173) | Exon 8 | 62.7 | 31.2 |
| 15. gRNAe8_2 | GGG | GTACACGATGATATCTGTTG (SEQ ID NO: 174) | Exon 8 | 62 | 31 |

Table 6B
Cas12

| | | | | On | Off |
|---|---|---|---|---|---|
| crRNA | PAM | Sequence (5′-3′) | Target | target score | target score |
| 2. crRNAe3_1 | TTTA | ATGATGCAAGACCTTGCAGG (SEQ ID NO: 304) | Exon 3 | | 96.9 |
| 3. crRNAe3_2 | TTTG | GCAAGGTCTTGCATCATGGG (SEQ ID NO: 305) | Exon 3 | | 87.7 |
| 4. crRNAe3_4en12 | TTTG | GAGACCCCATCATGGTGCTC (SEQ ID NO: 307) | Exon 3 | | 96.2 |
| 5. crRNAe3_5en12 | TTTC | AAAGCCAACTTCTCCATCGG (SEQ ID NO: 308) | Exon 3 | | |
| 14. crRNA e8 | TTTC | TCTGTGTGAAAACGCAGTGAT (SEQ ID NO: 275) | Exon 8 | 50.7 | |

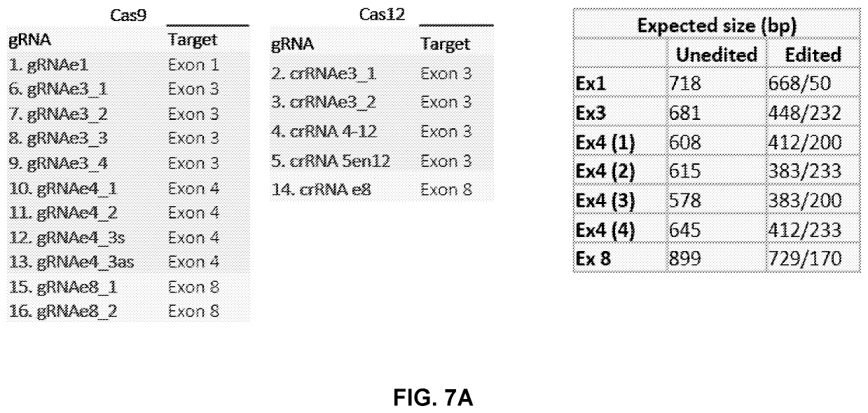

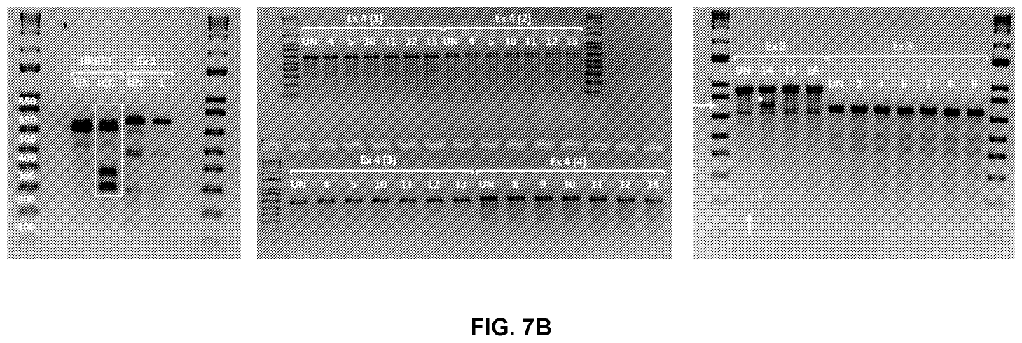

FIG. 7: KIR2DL2 knockout experimental design. A) 2×10⁵ human T cells known to express KIR2DL2 were nucleofected with both gRNAs or crRNAs targeting the housekeeping gene HPRT1 or different KIR2DL2 exons. Table on the right shows the expected size, in base pairs (pb), of the unedited or edited PCR products after T7E1 cleavage. B) Forty-eight to seventy-two hours after transfection, DNA was extracted, and every targeted exon amplified to assess Cas9/Cas12 cut by a T7E1 cleavage assay.

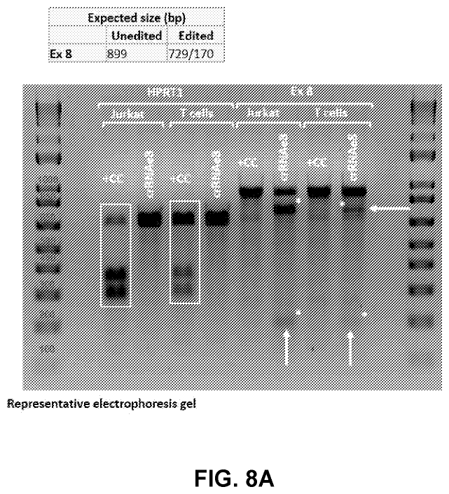

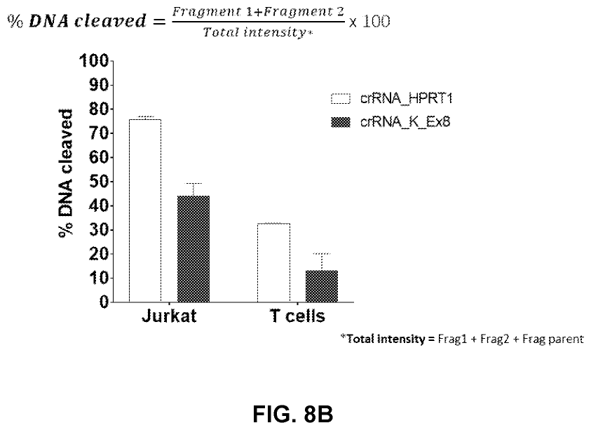

FIG. 8: T7 Endonuclease I cleavage assay confirmed KIR2DL2 exon 8 cleavage by the Cas12 nuclease. 2×10⁵ human T cells and Jurkat T cells, known to express KIR2DL2, were nucleofected with a crRNA targeting the HPRT1 gene or the KIR2DL2 exon 8. After 48-72 hours, DNA was extracted and cutting efficiency was assessed with a T7E1 cleavage assay. A) The expected size of KIR2DL2 exon 8 amplicon is 899 bp, whereas the edited cells shows two bands of 729 and 170 bp (white asterisks and arrows). B) Cleavage efficiency was calculated as the percentage of DNA cleaved by using the following formula:

(Fragment1+Fragment2/Total intensity)*100. Total intensity was calculated by the sum of intensities of the fragment 1, fragment 2 and fragment parent. Results shows an average efficiency of 45% in Jurkat T cells and 18% in human primary T cells. HPRT1 cleavage was measured as a nucleofection control. Bar represent the mean±SD of two independent experiments.

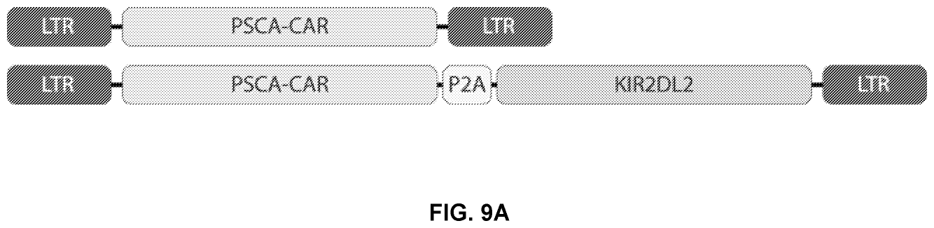

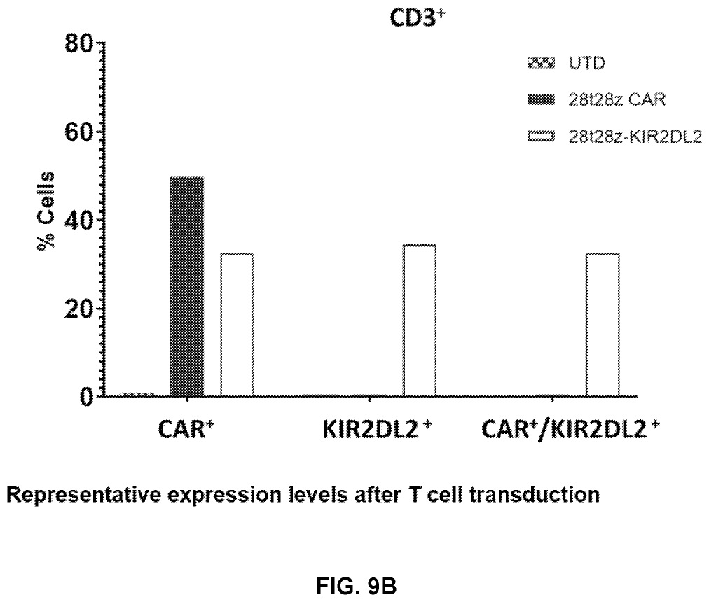

FIG. 9: KIR2DL2 bicistronic expression model. The MSGV1 retroviral vector containing the PSCA-CAR 28t28z followed by the KIR2DL2 CDS separated by a P2A peptide was designed and used for viral production. OKT3-stimulated PBMCs were transduced with viral particles containing both the PSCA-CAR 28t28z or the CAR+KIR2DL2, and the surface expression of both proteins were analyzed by flow cytometry 7 days after transduction. 9A shows the schematic representation of the retroviral vector. 9B shows a representative PSCA-CAR and KIR2DL2 expression 7 days after transduction. The P2A peptide allows the expression of both molecules at the same time in different transcripts with high efficiency. Untransduced cells (UTD) were used as a negative control.

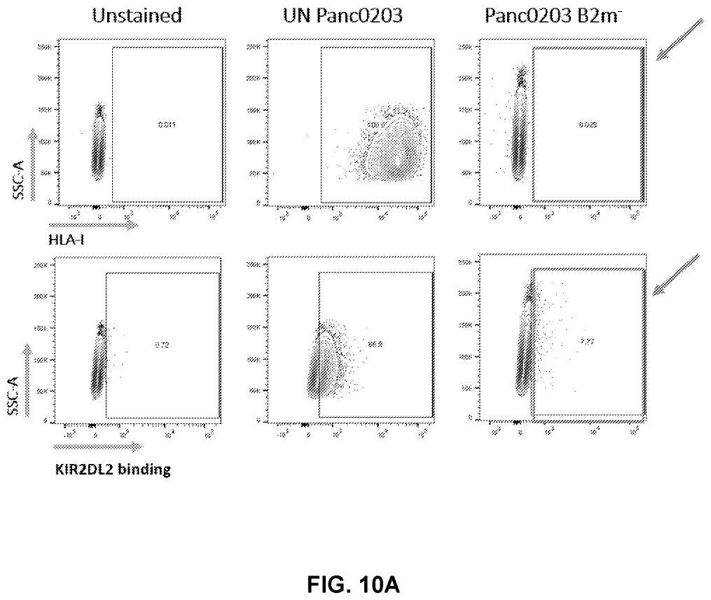

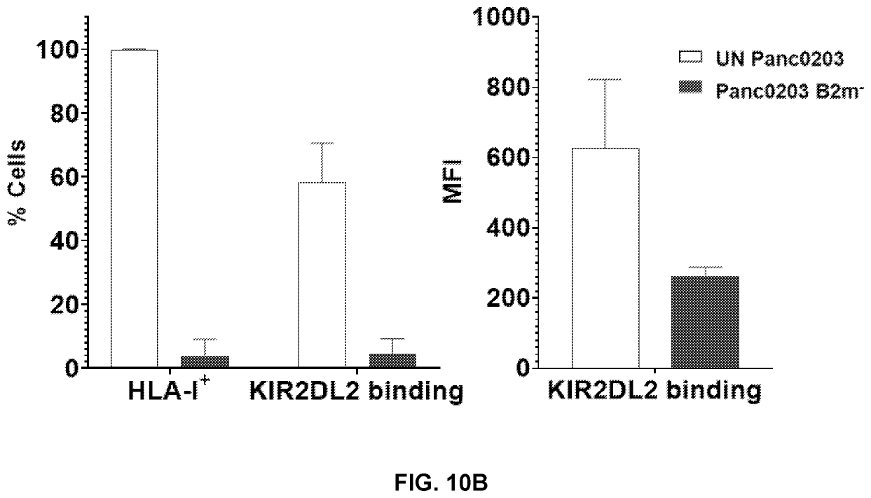

FIG. 10: HLA-1 deficiency impairs KIR2DL2 binding to Panc0203 tumor cells. KIR2DL2 expressed in the T cell membrane interacts with HLA-C1 expressed in the tumor cell membrane. We engineered a PSCA-expressing cell line (Panc0203) through CRISPR-Cas9 with a gRNA targeting the p32-micrglobulin gene to abrogate HLA-1 expression (Panc0203 β2-m⁻). Edited single cell clones were isolated and purified by FACS. HLA-1 expression and KIR2DL2 chimera (KKIR2DL2-Fc) binding were assessed by flow cytometry. A) Representative dot plot analysis showing HLA-I expression and KIR2DL2 chimera binding to unedited Panc0203 cells or Panc0203 β2-m⁻. B) Graphical representation of the percentage of cells expressing HLA-I and binding the KIR2DL2 chimera and the mean florescence intensity (MFI) of that expression and binding. Data is presented as a mean±SD of two independent experiments. Results shows that lack of HLA-I expression in tumor cells impairs KIR2DL2 binding. These cells will allow us to create an interaction model for the assessment of KIR2DL2 biology both in vitro and in vivo by comparing their ability to be killed by PSCA-CAR T cells expressing, or not, KIR2DL2.

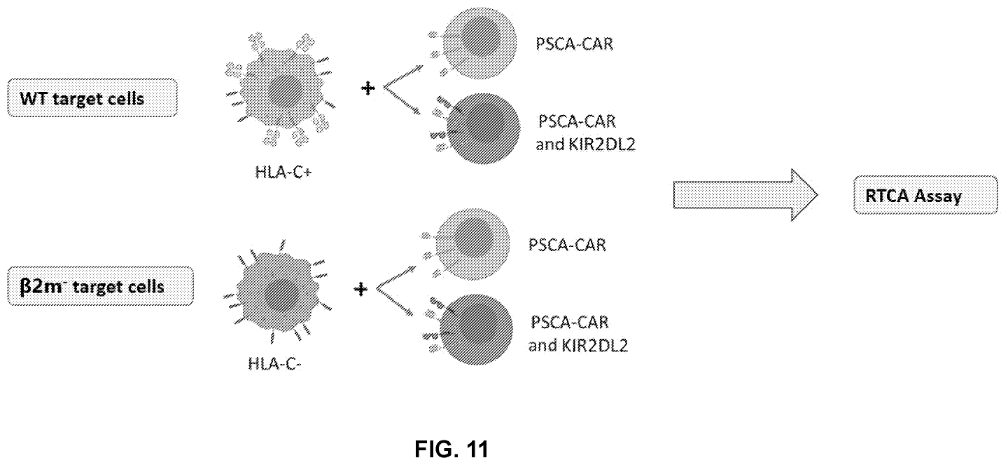

FIG. 11: Human T cells transduced to express the PSCA CAR (28t28z) or the PSCA CAR together with the KIR2DL2 protein were incubated with Panc0203 cells unedited or CRISPR/Cas-edited to abrogate their p2m expression, at different effector:target ratios.

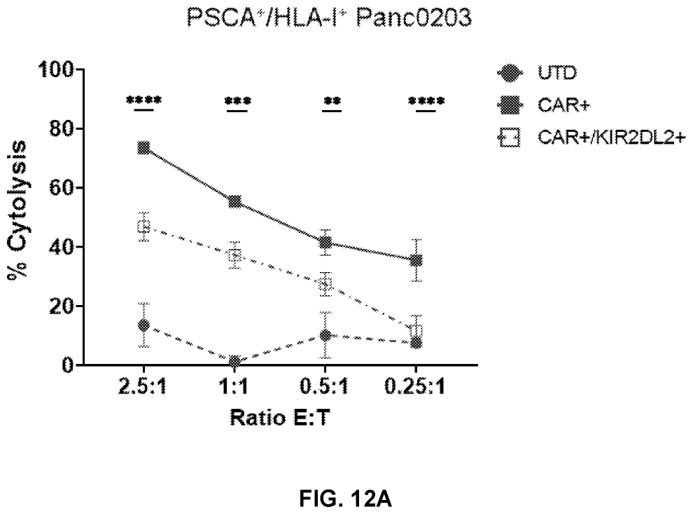

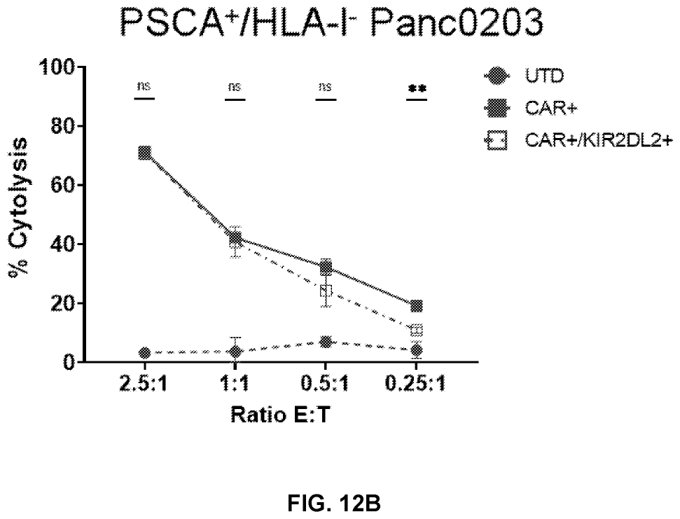

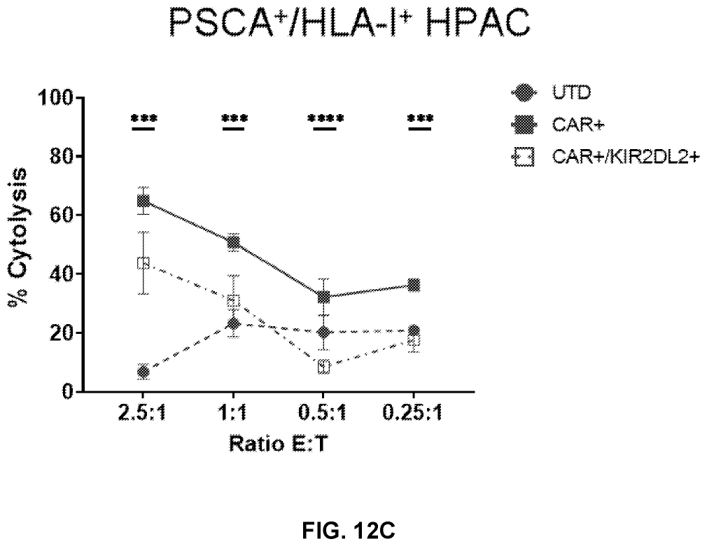

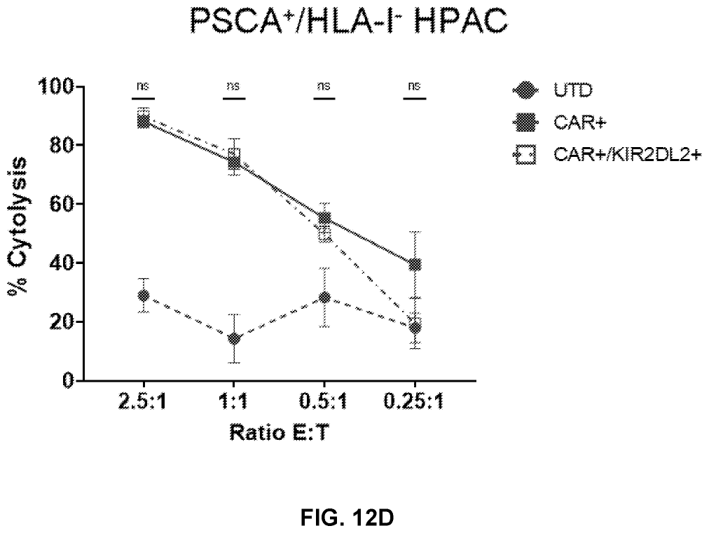

FIG. 12: KIR2DL2 impairs CAR T cell cytotoxicity in vitro against different tumor cells. Human T cells transduced to express the PSCA-CAR 28t28z or the PSCA-CAR together with the KIR2DL2 protein were incubated with Panc0203 cells unedited or CRISPR/Cas9-edited to abrogate HLA-I expression. Cytolysis was assessed by a Real Time Cytotoxicity Assay (RTCA) and percentage of cytolysis (% cytolysis) calculated using the RTCA software Pro (Agilent Technologies, CA, USA).

FIGS. 12A and 12B show the % cytolysis at different effector:target ratios (E:T) after 24 hours incubation with PSCA⁺/HLA-I⁺ Panc0203 cells (FIG. 12A) or PSCA⁺/HLA-1-Panc0203 cells (FIG. 12B). FIGS. 12C and 12D show the % cytolysis at different effector:target ratios (E:T) after 24 hours incubation with PSCA⁺/HLA-I⁺ HPAC cells (FIG. 12C) or PSCA⁺/HLA-I⁻ HPAC cells (FIG. 12D). Data are shown as a mean±SD of triplicates for every E:T ratio. Statistical significance was calculated with a two-way ANOVA test comparing the % cytolysis between the PSCA-CAR and the PSCA-CAR/KIR2DL2 T cells. Ns (non-significative); **** p<0.0001; *** p<0.0005; ** p<0.005; * p<0.05. For both tumor cells lines, KIR2DL2 interaction with its HLA-C ligand seems to significantly impair CAR T cell function, whereas cells that lack HLA-I expression are efficiently killed by both CAR T cells. These results suggest an inhibitory role of KIR2DL2 in CAR T cell biology, thus allowing us to target this marker as a therapeutical approach to enhance CAR T cell adoptive transfer therapy.

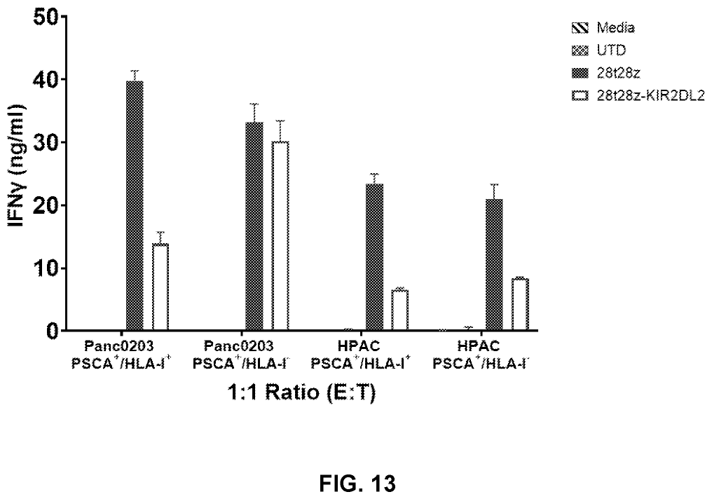

FIG. 13: KIR2DL2 expression impairs CAR T cell IFN-γ secretion. Human T cells expressing the PSCA-CAR or the PSCA-CAR together with the KIR2DL2 molecule were cocultured for 24 hours with both PSCA⁺/HLA-I⁻ or PSCA⁺/HLA-1-Panc0203 and HPAC tumor cells. Supernatants were collected and IFN-γ was measured by ELISA. Quantification of IFN-γ in wells with only tumor cells and media, or tumor cells and untransduced T cells (UTD) was used as a control. Data represents the mean±SD of three independent measurements. In accordance with the cytotoxic assay, CAR T cells lacking KIR2DL2 seems to produce more IFN-γ when cocultured with cells lacking HLA-I expression, thus suggesting a suppressive effect for the KIR2DL2 molecule.

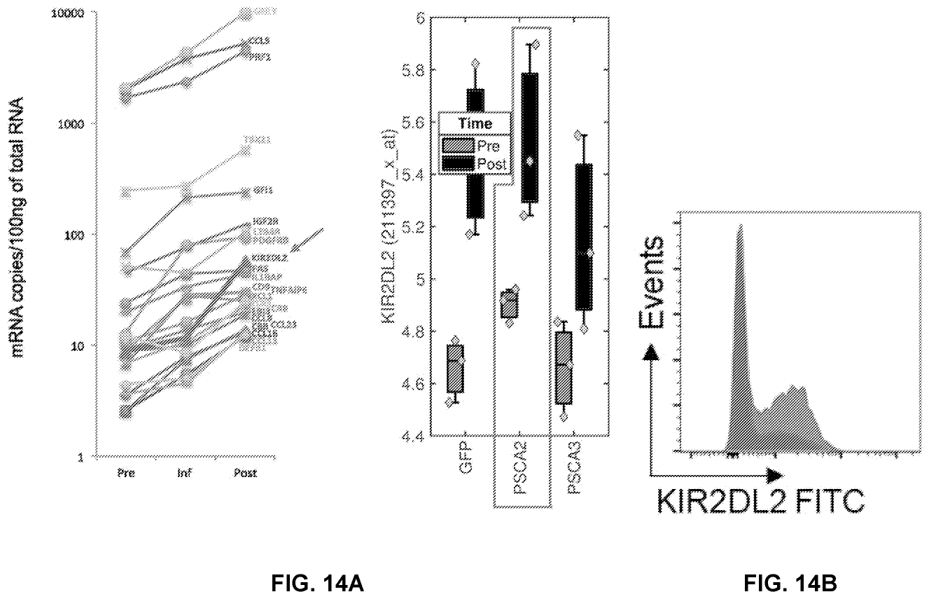

FIG. 14: KIR2DL2 mRNA expression is upregulated both in patients who received TCR-transgenic T cells and in PSCA-CAR T cells. A) Left panel. mRNA quantification of relevant genes for the immune system showing an upregulation in KIR2DL2 post-infusion, previously published by Dr. Abate-Daga [3]. Pre-infusion samples (Pre) are unmodified T cells; Infusion samples (Inf) are genetically modified T cells as they were administered to patients; Post-infusion (Post) cell are peripheral blood T lymphocytes collected 1 month post adoptive transfer. Right panel. Human peripheral blood T cells were genetically modified to express a second-generation CAR (PSCA2), a third-generation CAR (PSCA3), or GFP as a negative control. Following 14 days of expansion ex vivo, T cells were administered to immunodeficient mice bearing subcutaneous xenografts of HPAC pancreatic cancer cells. Thirty days after infusion, splenocytes were collected. The gene expression of these post-infusion T cells (Post) was compared to that of the infusion product (Pre). The box plot shows three independent replicates of the relative expression values for each condition, modified from Ramello and Benzaid, et al [4]. B) Flow cytometry analysis of PSCA2-tranduced cells confirms the upregulation of KIR2DL2 protein post-infusion.

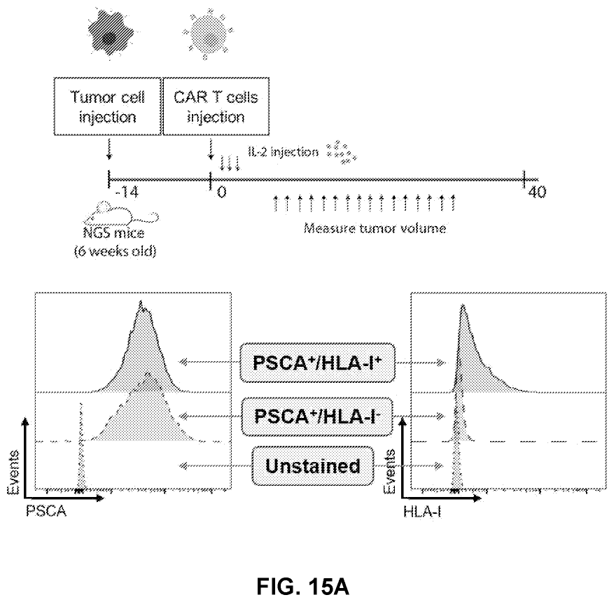

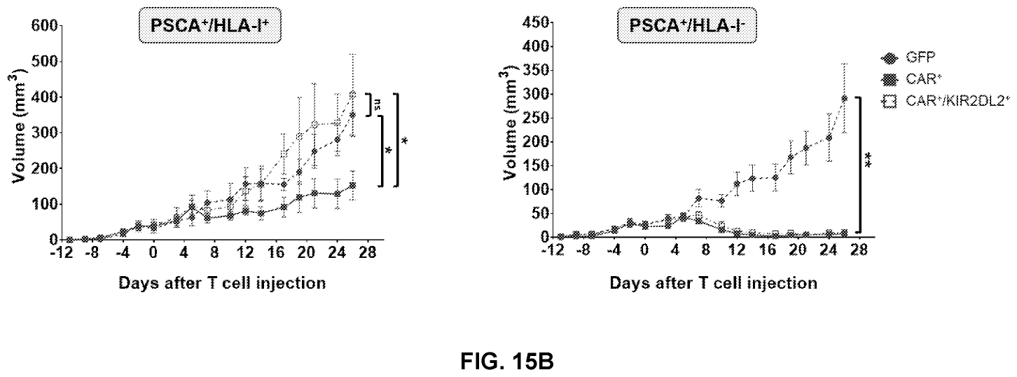

FIG. 15: KIR2DL2 impairs CAR T cell cytotoxicity in vivo. KIR2DL2 role in CAR T cell effector function was assessed in vivo using a NSG mouse model. A) Schematic representation of the in vivo CAR-T treatment protocol. HLA-I expressing (PSCA⁺/HLA-I+) or HLA-deficient (PSCA⁺/HLA-I⁻) tumor cells were injected into the flank of NSG mice. Mice were randomized into four groups (n=5 each group) and treated with 5×10⁶ PSCA-CAR or PSCA-CAR/KIR2DL2 T cells; GFP-transduced T cells were included as controls. Tumor size was measured by caliper three times a week. B) Tumor growth curve in each group was shown as mean±SEM. Linear regression analysis was used to calculate the tumor growth slope, and the statistical differences between the slopes were calculated using one-way ANOVA. Non-significant (ns); * p<0.05; ** p<0.005. As shown in vitro, KIR2DL2 expression together with the PSCA-CAR impairs T cell cytotoxic function in the presence of KIR2DL2 ligand, whereas the absence of its ligands allows the cells to properly react against target cells.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for enhancing anti-tumor activity of lymphocytes, comprising treating the lymphocytes with a KIR2DL2 inhibitor or genetically modifying the lymphocytes to inhibit or ablate KIR2DL2 expression.

2. A method, comprising:
(a) collecting lymphocytes from a subject with cancer;
(b) treating the lymphocytes with a KIR2DL2 inhibitor or genetically modifying the lymphocytes to inhibit or ablate KIR2DL2 expression.

3. The method of claim 1, wherein the KIR2DL2 inhibitor is an siRNA, antisense, gRNA, or crRNA oligonucleotide.

4. The method of claim 1, wherein the lymphocytes are genetically modified by inserting a chimeric receptor into the genome of the cell at a location that disrupts expression or activity of an endogenous KIR2DL2 protein.

5. The method cell of claim 5, wherein the chimeric receptor is a chimeric antigen receptor (CAR) polypeptide.

6. The method of claim 1, wherein the lymphocytes are selected from the group consisting of alpha-beta T cells, gamma-delta T cells, Natural Killer (NK) cells, Natural Killer T (NKT) cells, innate lymphoid cells (ILCs), cytokine induced killer (CIK) cells, cytotoxic T lymphocytes (CTLs), lymphokine activated killer (LAK) cells, and regulatory T (Treg) cells.

7. A therapeutic cell produced by the method of claim 1.

8. A method of providing an anti-cancer immunity in a subject, comprising administering to the subject an effective amount of the therapeutic cells of claim 6, thereby providing an anti-tumor immunity in the subject.

9. The method of claim 7, wherein the subject is HLA-C1+.

10. The method of claim 7, further comprising administering to the subject a checkpoint inhibitor.

11. The method of claim 10, wherein the checkpoint inhibitor comprises an anti-PD-1 antibody, anti-PD-L1 antibody, anti-CTLA-4 antibody, or a combination thereof.


SEQUENCE LISTING (311 sequences)

SEQ ID NO: 1 - protein, 348 aa, Homo sapiens
MSLMVVSMACVGFFLLQGAWPHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 2 - DNA, 14488 bp, Homo sapiens
cgcggccgcctgtctgcacagacagcaccatgtcgctcatggtcgtcagcatggcgtgtgttggtgagtcctggaagggaatcgagggagggagtgcggggatggagatcggggcccagagttggagatataggcctggaagtggagttatgggcctagagatggagtgatgggcctagaagtggagatctgggcctggagtggagatatgggcctggaggttgagatatgggcctgcagtagagatatgggcttgtagtggagacatgggcctggagatggagatatgggcctggagatggagatatgggcctgcagtagagataggggcctggagtggagatatgggcctggagtggagatatgggcctgaagtggagatatgggcctggaggtggagatatgggcctggaggtggagatatgggcctggagtggagatatgggtctggaggtggagatacgggcctgcagtagagatatgggcctggagtggagatatgggccaggagtggagttatgggcctagaggtggatatctgggcctggagtggagatatgggcctaggaaggagatatgggcctgggtgtggagatatgggactggagaggtgatatgggcctggagtggagatatgggcttagggtggagttctgggcctggggcggagatatgggactggattggagataggggcctagggtggagatctgagcctggattggcgatatgggcctagggtggaaatatcagcctggagtggagatatgggcttggggtggggatatgggcctggaaactgggtctctgcacagccgacagccctgttcttgggtgcaggtaggcactgagggtgagtttaacttcagcccaggaagggcctggctgccaagactcacagcccagtgggggcagcaagggagggctggttcgcctgcagatggatcgtccatcatgatctttctttccagggttcttcttgctgcagggggcctggccacatgagggtgagtccttctccaaaccttcgggtgtcatctccccacataagaggattttcctgaaacaggagggaagtcctgtcggggagtctctcataaactaggaagagaggaccctggggtgctcagcccacatttctgacctcgcctccctggcctctcaaccccttggcagagtcaagttctgtggggaccagggttagactggggtgctcaaagctggggtgtgtggttgggaagtggtaggaacagcagatcctctgaggacaaaggtgttactcacacacttcagcgtttccatgatggtaggggctgcagtgtggctgctgtcattctaccagaagaggtgggaaaccacagccatggccctgacattccaaatcctctgatgggggctcagttgtttattttcgttcaggcatccgctgatatccattcacaaaggacatgccctccacctcatgtctaccctgtgttgttttatgtgagtaatcttacagtatcaaaatctagtaggagtctctttactcagcacttgctcaaagttctcagctgaggcttttgttgtagggagacaccatgtctttgcgggatgggtccttccttcagccctgggcaccaaggtgtgatagtagccatagaaacgtggaaagcgaggagaatcttctgagcacagggagggaggggcagttccacatcctcctctctaaggcggcgcctccttctccccaaggtggtcaggacaagcccttgctgtctgcctggcccagccttgtggtgcctctaggacatgtcattcttcggtgtcactcttatcttgggtttaacaacttcagtctgtacaaggaaggtggggtgcctgtccctgagctctacaacagaatattctggaacagccttttcatgggccctgtgacccccgcacaacagggacatacagatgtcggggttcacacacacactcccccagtgggtggtcagcacccagcaaccccctggtgatcgtggtcataggtcagagggctcctgtcttggattctccttgtcccacctcctgaatcccagagcttctggtgggcatgtccttgagggtcccatcacgcaggccctgactgtatttgtggtaaagggggattgaatacagggaaatgggtgctgtggtgggaagaataattgtccccagtgatgactacattctaatccctggagtctgtgactatgtatgttataggggaagggactgaaggggaagatggagctcatggggagacagcctggactgtcccactgggctcagtgtaatcacaagggtgcacatgaaaggaggaggaagaggggagtggggattagagcagtccagtggaagtcttcaccagctttgaaggtggaggaaggccaagagccatgaatgcaggtggcctatagaggctggaaaagtcaaggaactgattctccagagtctccagagggaacaaagccctgcagatgccttgattttagcccaggaaaaatagggtccaatttctgtctccagtactggaaggtgtcagtgtggtctctcctgcttccatgcttctgataattttgtacagcagcaacaggaaaccaacactggaacccaggtcaaggacaagttaagaaacaacccaaggaaagccaggcatggtggcaggcgcatgtaatcctagcgactcaggaggctgagggcaggagaatcacttgaacccaggaaacagaggttgcagtgagcctagaccacaccacttcactccagcctgggtgaaggagtgagactctgtctccaaaattaattaattaattaaagaaaccaaagaaggagaaggttggctaccctgagatcagcaagggtgggatgatgatgccaccaccaggctccatccacatagggaggggttgatactcctccaaccagcaccaggagccagcctatggaagctggcaccatggagaaggcacaggcatggcaagagtggctcccagtccccaccaggaacagggtgtgtggacactggtgcctgccttattcatcagttcatatcttctgccaaggattgcaattcatccaaaagagattgaaccaggctgataagagcctggatgtgcagcctatcctggttcctctttcacccccacataaacagcaggaaagacattagtgtgaaatagatacaacaccccaagagatgaggctaagcccagtgggaagggaatcagaggctactagagacagagggacagagaagagggagggagacagatggaaggacctgcaccaggagttaagggcacagaaaagaacatgaagacacagagaggaaggagagagacagacaccagcaaggggaagcctcactcattctaggtgccatggatgggatgataaagagagacaccttctaaactcacaacctctcttcctaggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtgagagtgtccggacattctcattgtcattgggctgcagagtgaatgatccacgacttggaacccccaggtagttgtaaggaagatgagcttggtattcttatggagagagactgacttgctgaggtttgtaccaacagagacagagaaacaggagacacaagtacagaccaggtgtcataacggaggacagacacaggggccatacagggagttagaaaagacagaaagagttaaaagagacagacagacagacatgtcccagagagaggtgtccctccatgctgactttgctcacagacctggcacaggttagaagtttcatttctgttttacctccacaaagtgttctctaccaggagaacccaaggacacccatatttctgacctgagttgggccctgtggcctcaggccttgtggcacctacaggccatgtttattctgacacctctgccttccatgtaatggagagtaaccgtcccaggatatcatggccccagaacaccaacccctgtatgctgtgtgaacttgtggtctccagactggattctgaggctcacattccaaataaccccacatatgaaaggatcactgagaggcacagagaaaaatcaggaacaccaaaaagcaaagacataaacacacggagaatgagccagaggaaggagattgagagactcacagacacataaagagagagaaaagagggcagaggagtggtgagaatgatggcagggagcagagaaaagcactaaaattagagtcctgagagagaggcacaaggacatagaaacatggagatgtggggatgaattgcagagattccaaagagagctagagagaccgagaggcagagcaatacagatgatagatggatagatatagatagatgataaataggtagatgatagataataggttaaagatacatagatgatgattgattgattcattaatagataatacatagagatgatgatgatgaagacagataatacgtacagatagagaggcagacagaaatcatagagagagagatgatacatacatataaataacagatgattgatggatagatagacaactgatagatacatagatgatatatagatatagatgacaggtagagaatttgtagataggcaccgaatagataaatagatagatcgacagataatagatagaaatatgcagaaagttatgaacaggacacaacgtgagaaacttagaatttaaaaaagtaacatcaagtcaaccaacccaaggagagtcagagagaataaaacaatccaaaaacggaaaacatatctagaggtggggaagcgaggtcagagacctagagagacagagaaggtggaagaaggaaatagatatgaagagagatggggtggagggtgagagagagagagagagagcattaggtcatagagcaggggagtgagttctcagctcaggtgaagggagctgtgacaaggaagatcctccctgaggaaaatgcctcttctccttccaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcataggtgaggaaaccccatatctgtctcatgtcctatgatcctagagccttagctgaggagcttcctgctgatgatggagataagcatggacagatgcagagagaagacgaagcttgggtgtgagggagggatcagggcacaggatggcagacagggcacctccaaaccctcctacacggcctgcatgaaggcccgcggccagggctccaggcacacaggcagatggagaaagcggtcaggagagacccagaggagggagactgggctcagtttgggaagatcagaggttccctcagcccctcaacattacccatttcccagaagcccatcctggcctctcacccacacagggatgtcatcaccagcaacccctacaccctttacttttgtttgaagaaatatttattgaggataaatatacctatatagcttaccacctttaacattttttttttttttgaggcagagtctagctctgtcccctatgctgcagtgcagtggcacaatctcagctcactgcaacttccgcctcctgggttcaagtgattctcctgcctcagccacctgagtagctggtgctacaggcgcgcaccaccacgccaggctactttttgtatttttagtagagaggtggtttcaccatgttggtcgagctggtctccaactcctgaccacgtgatccacccgcatctgcctcccaaagtgctgggattacaggcatgagccaccactcccagccacatttaccatttttaagtgtaaagtctagtggtcataaatacatttataaatatatatatatatatatgtatgtatatatatatatatatatatatatatatatatatttttttttttttttttaccctccacccttttcttcctggcctctggaagccaccattctactctctaccttcatgagatccaccttttagctctgtatatgggtgagaaatgggaatctttgtaatgacttgcagttccatccatgtggctgcaaatatcaggatgttattctttctatggatgagtagtctccactgtgcgtatgtactacattctctctatccattcatccactgatgggcaggtaggttgactccacatcttggctactgtgaacagtgctgcaccaatcatacgagtgcagatatcacttcgatatattgatttactttcctttggatataaacccagtagtgaaattgctggatactatgaaagttctctttttagttattcgtttgttgttttgtttttgtttttgagacagtttccctctgtgcccaggctggagtacaagtgatgtcatcttggctcattgcaacctctgcctcctgggttcaaatgattttcctacctcagcctccctagtagctgggattacaggtgcacgccaccatgcctggctactttttggtttttttagtatagatggggtttccccatgttggctgggctgctctcaaactcatgacctcaactgaggtgtccgcctcggtctcccaaagtgccgggattacaggcatgatccacctcacccaacctctttttagttctttaaaggacttccacacttttctccgtaaaggctgtactaatttacactcctaccaacagggtattagggttctcctttctctaccactttggcaggatttcctttgcctgtcttgcagctaaaagccattttactttatttcattttattttgagatggagtttcgctcttgtcacccaggctggagtgcagtggtgcgatctcggctcaccacaacctccacctcccaggttcaagcgattctcctgcctcagcctcccgagtagctggaattacaggcacacgccaccacgcccgactaatttttgtatttttagtagagacagtgtttctccatgtgggtcagactggtctcaaactcccgaccttatgagattcacccacctcaggctctcaaagatctaggatgacagacgtgagccaccacgcccggcctaaaagccattttaatggggtgagatgaaaactcactttgattttaatttgcgtttctctgatgatgagtgatactgagcagtttttcgtatgtggggaaatttcatgtcttttgctcctgtttcaattaaatcatttgttttattgagttgtttgagcttcttatatttctagttattaatcccatctcagatgcatagtttgcacatatttgctcccaatctgtgggttgtctcttcactttgttggtttatttttagcggtgcagaagttgcttagcttgaggtaatcccaatggtctatttttgcttcgattacttgtgttttgaaggtttaaaacaaaatgtcttccttcagacaaatgtcctggagcatttccccaatattttcttctacgtgtttcataggttcaggccttagactcacatctttaatccattttcatttgatttttgtgtatggtgacaggtagaggtgcagtttcattcctctgcatgtagatgtccaggtttccctgcactgtttattgaaaagactgtcctttcctgattgtgagttcttggcacctttgtcaaagtccattggatgggctgggcatggtgactgacacctgcaatttcagcactttgggagcccaaggcgggtggatcacctgaggccaggagttcaagattagtctggccgacgtgatgaaacatcgtctccactaaaaatatataaattagctgagcatggtggtcagcacctataataccactactcaggagtttgaggccagagaattgattgaacccaggaggctgtggtggcagtgaaccgagattgcacctctgcactccagcctgggtgacagagcgagactccatctcaaaagaaaaaagaaaaaaacattggatgtaaatgcatggattatatttgtgttgttcattctgctccattgttctatgtgcctttcttcatgccaacatcatgctgtcttgcttactacagctctgtaacatattttgagatcaggtagtgtgatgctcctgttttctctttataccttgaagtctcaagacaatgggcgtcacatacaaaaattatggaaaaaaggatcccaggactcccagggcccaatattagataacagagtgttggccatgaaccaacctcaaagatttccattgagtagaggacagacaccctcatttcctcacctctctcctgtctcatgttctaggaaacccttcaaatagttggccttcacccactgaaccaagctctaaaaccggtgagtacagaaccctcttatatccgcttttggaaacctggggaggtagaaaccttcgatgcaggcattgactcagcatctcgcagctctgacattgtacgcctgtcttctaccatctccgaactccagatactccaacagcgaaagggatctgggcccaacctagggctcagtgaaatctcttaatctctcattttatggagctgagacctcctacaagctagaagaatgattgccaatctgacatccttctcaggaaaaatgcaatgtttgttctgcctgcattcctaactggaggataaattcctgggggcttgagagagggaagggaagggaacatctgatgagggcgaggtgttttagagaagttccacttgccaaggaatgaattactgttggtcatgaagcaaccctggctgactcagcagagcaacagccttgccgtaacagagaacggagctcatgcacgcacacttcgactcactgactcattcagccacggccccatgctcaggctgtgcagtgcggaaccttttcctattgttgccataacaaatttccacaagattcgtgggtgaaaacaaaacggttttttaattatcttacagtgctgtagctcaaagtaggaagtgcatcttactgggctaaaatcaaggtgacagcaaggctgccttccctctgaggattccaggcaagaatctgcttctcacttgtcccagcttctaaaggctcccagttccttggctcctggtccccttcctccttcctcaaaacccacaaagactggtcacatctcacatggcatcactcagtgccttcttccttaccacacctctttctctgaatgctgctctcccttcttccttatcttttgaaaacttggggattctattgggttcaccaagatgaaaatccctcataatctcctggaaatcatccaggatacccttgttttaagttcagctgattagcaaccgtaattccatctacaatcttcattcctcctttccatgtaaaataacatattcacaaggtatggaggctaggacagggacattttggggtgggacagcattctcctgccttccacaaacagtgaacaagatgcatttggcctctgcccttgggacactgatattgcagatggttaaatgggagggcagaaaatgaatgcacaagtggatctataaatgaatgatccattgggaagcatctgtgcatgaaatctattttttgtttgttcttttgtttattgagacagagttgccctctgtcttccaggctacagtgcagtgtcacgatcttggctcactgcaacctgcttctcctggattcaagtgattctcctgcctccgcctctcgagtagctgggattacaggcaactgccaccgtgcccggctaattctttttgtatattttttgtagagaggatgtttcaccacgttggccaagcttgtctgaaactcccaacctcaagtgatccgaccgtctcagcatgccaaagtaatgggactacaggcgtgagccactgtgcccagccagaattcaaaatcaataatagataatgctgagtgtatgatttcaggtgacaaagaaggtctcactattcagatatttgtgacattaatgaaaaacacggattgaacccctgaaagattggcggaaggattttgcacacacagctgtcagccgtgaaggcacaaaggtgaaaacaatctgatgtggaaggaagaggctcttcctcaaatgctgggaatgatgtggggagaatgacaagatgactgtggagagacggagagcacactgggtacacaggaaactaaggaggaacaaggagtgtgtgtttgacactcacagccattggattcacctcggggtagccaggaatccctacatgattaatatgactgacatgaaaataagggaggctcagttgcataactggaatctaggagaccgtggaaaaggcaattgccgccccactggtgaaatgtggtgctgatttagacactaaatgaatgaagtagatggatataagataggtttgtgaggtagaatcattgactggaaaggcttgctgggtttgattttcctacttgtttaatcctcgcttaattaatttctttctgagatttattcatcctacacataaatcaatacctggcaaaggagtgacagatatatgaggggtggtggaaatgaagagacctattatagcataatatacaagtctgtgaacggtggctcacgcctgtaacccagcactgcaggaggccaaggcgggtggatcacatgaagtcagcagttcgagaccagcctggccaacatggtgaaaccctgtctctaggaaaaacacaaaaattagccgagcatggtggtgcatccctgtaatcccagctcctactctggaggatgaagcaggagaatgacttcaacccaggaggtggaggttgcagtgagtggaggttgcatcactgcactccagcctgggtggcacaaggagactccgtctcaaaaaataaaaataagaaatgcataaatataaatataatataacacacgcaaatgacaaagggacctgaattccaatcatgatttttctatttctctataattacttctttgatcctttatcttatccattaggcaatgagcctaaaacctcttccctatttggctttctgtgagcatgagatcatatagaaaatgtgaaagtccgctgaatcctccagcacagatcctggaatagagaaagtgctctggtcatcacaaaaaaaacttgcccactcacccaaatcccccacctcacccctacttccaatcacctgtggagattcaggtagaccatggggaggtaaacattaacactccttggagtgagtccagatcttggaatcagagatcagcgacagcactagctcctgctcccctttcctactaattcacaggaggacaggtggtattgaagcaatagatggccgagggggtggtccttcccccagcctctcgggtagaacagcagcctaatatgtgtctcccgagatcacaaagagcagcaggtttcacacgggcttcaacactatttcctggccgtttgacataagagaattctatttcgctttttttatcttgatttcacttttgttttctttccttggagaatgcaagttgtttgattcaagaatgctgtggatgtagaaaccctaaagcacattcgctgtgaatcaatcccagtccagtcttcccagagaagactctaaacacctcctggactgcacctgggcctatgccaattcctatcactcaccgtcactccagggagacagaacacacagagaatacgttacataggcaggttcattactaacagataagcagcgagtgacaacagaaacctatatttcaatgtgagccagtccctcaaggctcagaaaagctcctcgggacatatggagtcaccccatttgcagtgtagctgcgggaagccagaaagcagcccagcctgggttttgtaccctggagccacaggaagcactcagctaaagcactgcatgacgtcctccaggaagaacaggaagacagcccagggtgttctgagacgttcctcctgatctcaggaagttgctgtcttaggccatttttgttgctctaaaggaacacttgagcctcggtaacttctaaagaaaagagattggtttgcctcaccgttctgcaggctgtactggaagcatggcaccagcatctatttctcgtgacggcctcaggctgctcccactctggcagaagggaaggagggtctgtctgtgcagagaccacagagatcacacggcaagagagggagcaagggggagggggagtgatggagcttccaagctctttttaacaaccagctctccgggaactaatagagggggaacttgctaaccccgtctccttgggacagcattgatgtgttcatgatggatccacctccatgacccaaacacctctcaagaggcccaacctcccacagtgggggtgaaatttcaatgtgaggtttgaaggggtcaaacatctcaactaaagtagtcgtatcctcagcacgttctatggttactatgagagctataactgaaaaagcaggagaaagctgggtctcctgccatctgggtgcttgtcctaaagagatgttttatgtggttacctgtcaatcaagaaatgcgagacaattcataaagaggaactgctaagattagcttcttattggtgtctcatcttcttccaggtaacccccgacacctgcacattctgattgggacctcagtggtcatcatcctcttcatcctcctcttctttctccttcatcgctggtgctccaacaaaaaaagtaagtctcacgaagcagaggccagagagctcagggccatgtggggaagcaggatgggagcactcaggtgtgtgttcctcacaaacaggatggtccctggcccaaggcagcagccacagaggcaggactttctagagagggcaccagactccctgcccctgccttcaactcacagaccgttgcctgattctgaactgtatcctcatgtcccctgcagccactcacatccaggagaaggttccatgacaggcagaaagtgggagacagaatcaatgggatgggaactcagagctattcatgggatgggtccttgagctcagagagatagaatgtctgagtctgctgttggcaactgagggacctcagccacctatggtctccccctgtatgttggtatctgcttatgaaatgaggacccagaagtgccctccgagctgttttgttgacttccgtctcctacagatgctgcggtaatggaccaagagtctgcagggaacagaacagcgaatagcgaggtaggtactcctcggcccgggctcgtggctactgttattcccaaagagtcctggaaaatgtgagcaccctccctcactcagcatttccctctctccaggactctgatgaacaagaccctcaggaggtgacatacacacagttgaatcactgcgttttcacacagagaaaaatcactcgcccttctcagaggcccaagacacccccaacagatatcatcgtgtacgcggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 3 - DNA, 14670 bp, Homo sapiens
gagcacccactgggcctcatgcaaggtagaaagagcctgcgtacgtcaccctcccatgatgtggtcaacatgtaaactgcatgggcagggcgccaaataacatcctgtgcgctgctgagctgagctggggcgcggccgcctgtctgcacagacagcaccatgtcgctcatggtcgtcagcatggcgtgtgttggtgagtcctggaagggaatcgagggagggagtgcggggatggagatcggggcccagagttggagatataggcctggaagtggagttatgggcctagagatggagtgatgggcctagaagtggagatctgggcctggagtggagatatgggcctggaggttgagatatgggcctgcagtagagatatgggcttgtagtggagacatgggcctggagatggagatatgggcctggagatggagatatgggcctgcagtagagataggggcctggagtggagatatgggcctggagtggagatatgggcctgaagtggagatatgggcctggaggtggagatatgggcctggaggtggagatatgggcctggagtggagatatgggtctggaggtggagatacgggcctgcagtagagatatgggcctggagtggagatatgggccaggagtggagttatgggcctagaggtggatatctgggcctggagtggagatatgggcctaggaaggagatatgggcctgggtgtggagatatgggactggagaggtgatatgggcctggagtggagatatgggcttagggtggagttctgggcctggggcggagatatgggactggattggagataggggcctagggtggagatctgagcctggattggcgatatgggcctagggtggaaatatcagcctggagtggagatatgggcttggggtggggatatgggcctggaaactgggtctctgcacagccgacagccctgttcttgggtgcaggtaggcactgagggtgagtttaacttcagcccaggaagggcctggctgccaagactcacagcccagtgggggcagcaagggagggctggttcgcctgcagatggatcgtccatcatgatctttctttccagggttcttcttgctgcagggggcctggccacatgagggtgagtccttctccaaaccttcgggtgtcatctccccacataagaggattttcctgaaacaggagggaagtcctgtcggggagtctctcataaactaggaagagaggaccctggggtgctcagcccacatttctgacctcgcctccctggcctctcaaccccttggcagagtcaagttctgtggggaccagggttagactggggtgctcaaagctggggtgtgtggttgggaagtggtaggaacagcagatcctctgaggacaaaggtgttactcacacacttcagcgtttccatgatggtaggggctgcagtgtggctgctgtcattctaccagaagaggtgggaaaccacagccatggccctgacattccaaatcctctgatgggggctcagttgtttattttcgttcaggcatccgctgatatccattcacaaaggacatgccctccacctcatgtctaccctgtgttgttttatgtgagtaatcttacagtatcaaaatctagtaggagtctctttactcagcacttgctcaaagttctcagctgaggcttttgttgtagggagacaccatgtctttgcgggatgggtccttccttcagccctgggcaccaaggtgtgatagtagccatagaaacgtggaaagcgaggagaatcttctgagcacagggagggaggggcagttccacatcctcctctctaaggcggcgcctccttctccccaaggtggtcaggacaagcccttgctgtctgcctggcccagccttgtggtgcctctaggacatgtcattcttcggtgtcactcttatcttgggtttaacaacttcagtctgtacaaggaaggtggggtgcctgtccctgagctctacaacagaatattctggaacagccttttcatgggccctgtgacccccgcacaacagggacatacagatgtcggggttcacacacacactcccccagtgggtggtcagcacccagcaaccccctggtgatcgtggtcataggtcagagggctcctgtcttggattctccttgtcccacctcctgaatcccagagcttctggtgggcatgtccttgagggtcccatcacgcaggccctgactgtatttgtggtaaagggggattgaatacagggaaatgggtgctgtggtgggaagaataattgtccccagtgatgactacattctaatccctggagtctgtgactatgtatgttataggggaagggactgaaggggaagatggagctcatggggagacagcctggactgtcccactgggctcagtgtaatcacaagggtgcacatgaaaggaggaggaagaggggagtggggattagagcagtccagtggaagtcttcaccagctttgaaggtggaggaaggccaagagccatgaatgcaggtggcctatagaggctggaaaagtcaaggaactgattctccagagtctccagagggaacaaagccctgcagatgccttgattttagcccaggaaaaatagggtccaatttctgtctccagtactggaaggtgtcagtgtggtctctcctgcttccatgcttctgataattttgtacagcagcaacaggaaaccaacactggaacccaggtcaaggacaagttaagaaacaacccaaggaaagccaggcatggtggcaggtgcatgtaatcctagcgactcaggaggctgagggcaggagaatcacttgaacccaggaaacagaggttgcagtgagcctagaccacaccacttcactccagcctgggtgaaggagtgagactctgtctccaaaattaattaattaattaaagaaaccaaagaaggagaaggttggctaccctgagatcagcaagggtgggatgatgatgccaccaccaggctccatccacatagggaggggttgatactcctccaaccagcaccaggagccagcctatggaagctggcaccatggagaaggcacaggcatggcaagagtggctcccagtccccaccaggaacagggtgtgtggacactggtgcctgccttattcatcagttcatatcttctgccaaggattgcaattcatccaaaagagattgaaccaggctgataagagcctggatgtgcagcctatcctggttcctctttcacccccacataaacagcaggaaagacattagtgtgaaatagatacaacaccccaagagatgaggctaagcccagtgggaagggaatcagaggctactagagacagagggacagagaagagggagggagacagatggaaggacctgcaccaggagttaagggcacagaaaagaacatgaagacacagagaggaaggagagagacagacaccagcaaggggaagcctcactcattctaggtgccatggatgggatgataaagagagacaccttctaaactcacaacctctcttcctaggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtgagagtgtccggacattctcattgtcattgggctgcagagtgaatgatccacgacttggaacccccaggtagttgtaaggaagatgagcttggtattcttatggagagagactgacttgctgaggtttgtaccaacagagacagagaaacaggagacacaagtacagaccaggtgtcataacggaggacagacacaggggccatacagggagttagaaaagacagaaagagttaaaagagacagacagacagacatgtcccagagagaggtgtccctccatgctgactttgctcacagacctggcacaggttagaagtttcatttctgttttacctccacaaagtgttctctaccaggagaacccaaggacacccatatttctgacctgagttgggccctgtggcctcaggccttgtggcacctacaggccatgtttattctgacacctctgccttccatgtaatggagagtaaccgtcccaggatatcatggccccagaacaccaacccctgtatgctgtgtgaacttgtggtctccagactggattctgaggctcacattccaaataaccccacatatgaaaggatcactgagaggcacagagaaaaatcaggaacaccaaaaagcaaagacataaacacacggagaatgagccagaggaaggagattgagagactcacagacacataaagagagagaaaagagggcagaggagtggtgagaatgatggcagggagcagagaaaagcactaaaattagagtcctgagagagaggcacaaggacatagaaacatggagatgtggggatgaattgcagagattccaaagagagctagagagaccgagaggcagagcaatacagatgatagatggatagatatagatagatgataaataggtagatgatagataataggttaaagatacatagatgatgattgattgattcattaatagataatacatagagatgatgatgatgaagacagataatacgtacagatagagaggcagacagaaatcatagagagagagatgatacatacatataaataacagatgattgatggatagatagacaactgatagatacatagatgatatatagatatagatgacaggtagagaatttgtagataggcaccgaatagataaatagatagatcgacagataatagatagaaatatgcagaaagttatgaacaggacacaacgtgagaaacttagaatttaaaaaagtaacatcaagtcaaccaatccaaggagagtcagagagaataaaacaatccaaaaacggaaaacatatctagaggtggggaagcgaggtcagagacctagagagacagagaaggtggaagaaggaaatagacatgaagagagatggggtggagggtgagagagagagagagagagcattaggtcatagagcaggggagtgagttctcagctcaggtgaagggagctgtgacaaggaagatcctccctgaggaaaatgcctcttctccttccaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggtgaggaaaccccatatctgtctcatgtcctatgatcctagagccttagctgaggagcttcctgctgatgatggagataagcatggacagatgcagagagaagacgaagcttgggtgtgagggagggatcagggcacaggatggcagacagggcacctccaaaccctcctacacggcctgcatgaaggcccgcggccagggctccaggcacacaggcagatggagaaagcggtcaggagagacccagaggagggagactgggctcagtttgggaagatcagaggttccctcagcccctcaacattacccatttcccagaagcccatcctggcctctcacccacacagggatgtcatcaccagcaacccctacaccctttacttttgtttgaagaaatatttattgaggataaatatacctatatagcttaccacctttaacattttttttttttttgaggcagagtctagctctgtcccctatgctgcagtgcagtggcacaatctcagctcactgcaacttccgcctcctgggttcaagtgattctcctgcctcagccacctgagtagctggtgctacaggcgcgcaccaccacgccaggctactttttgtatttttagtagagaggtggtttcaccatgttggtcgagctggtctccaactcctgaccacgtgatccacccgcatctgcctcccaaagtgctgggattacaggcatgagccaccactcccagccacatttaccatttttaagtgtaaagtctagtggtcataaatacatttataaatatatatatatatatatatgtatgtatatatatatacacacacatatatatacatatatatatgtgtatatatatatatatatatatatatatatatatatatatatatttttttttttttttttaccctccacccttttcttcctggcctctggaagccaccattctactctctaccttcatgagatccaccttttagctctgtatatgggtgagaaatgggaatctttgtaatgacttccagttccatccatgtggctgcaaatatcaggatgttattctttctatggatgagtagtctccactgtgcgtatgtactacattctctctatccattcatccactgatgggcaggtaggttgactccacatcttggctactgtgaacagtgctgcaccaatcatacgagtgcagatatcacttcgatatattgatttactttcctttggatataaacccagtagtgaaattgctggatactatgaaagttctctttttagttattcgtttgttgttttgtttttgtttttgagacagtttccctctgtgcccaggctggagtacaagtgatgtcatcttggctcattgcaacctctgcctcctgggttcaaatgattttcctacctcagcctccctagtagctgggattacaggtgcacgccaccatgcctggctactttttggtttttttagtatagatggggtttccccatgttggctgggctgctctcaaactcatgacctcaactgaggtgtccgcctcggtctcccaaagtgccgggattacaggcatgatccacctcacccaacctctttttagttctttaaaggacttccacacttttctccgtaaaggctgtactaatttacactcctaccaacagggtattagggttctcctttctctaccactttggcaggatttcctttgcctgtcttgcagctaaaagccattttactttatttcattttattttgagatggagtttcgctcttgtcacccaggctggagtgcagtggtgcgatctcggctcaccacaacctccacctcccaggttcaagcgattctcctgcctcagcctcccgagtagctggaattacaggcacacgccaccacgcccgactaatttttgtatttttagtagagacagtgtttctccatgtgggtcagactggtctcaaactcccgaccttatgagattcacccacctcaggctctcaaagatctaggatgacagacgtgagccaccacgcccggcctaaaagccattttaatggggtgagatgaaaactcactttgattttaatttgcgtttctctgatgatgagtgatactgagcagtttttcgtatgtggggaaatttcatgtcttttgctcctgtttcaattaaatcatttgttttattgagttgtttgagcttcttatatttctagttattaatcccatctcagatgcatagtttgcacatatttgctcccaatctgtgggttgtctcttcactttgttggtttatttttagcggtgcagaagttgcttagcttgaggtaatcccaatggtctatttttgcttcgattacttgtgttttgaaggtttaaaacaaaatgtcttccttcagacaaatgtcctggagcatttccccaatattttcttctacgtgtttcataggttcaggccttagactcacatctttaatccattttcatttgatttttgtgtatggtgacaggtagaggtgcagtttcattcctctgcatgtagatgtccaggtttccctgcactgtttattgaaaagactgtcctttcctgattgtgagttcttggcacctttgtcaaagtccattggatgggctgggcatggtgactgacacctgcaatttcagcactttgggagcccaaggcgggtggatcacctgaggccaggagttcaagattagtctggccgacgtgatgaaacattgtctccactaaaaatatataaattagctgagcatggtggtcagcacctataataccactactcaggagtttgaggccagagaattgattgaacccaggaggctgtggtggcagtgaaccgagattgcacctctgcactccagcctgggtgacagagcgagactccatctcaaaagaaaaaagaaaaaaacattggatgtaaatgcatggattatatttgtgttgttcattctgctccattgttctatgtgcctttcttcatgccaacatcatgctgtcttgcttactacagctctgtaacatattttgagatcaggtagtgtgatgctcctgttttctctttataccttgaagtctcaagacaatgggcgtcacatacaaaaattatggaaaaaaggatcccaggactcccagggcccaatattagataacagagtgttggccatgaaccaacctcaaagatttccattgagtagaggacagacaccctcatttcctcacctctctcctgtctcatgttctaggaaacccttcaaatagttggccttcacccactgaaccaagctctaaaaccggtgagtacagaaccctcttatatccgcttttggaaacctggggaggtagaaaccttcgatgcaggcattgactcagcatctcgcagctctgacattgtacgcctgtcttctaccatctccgaactccagatactccaacagcgaaagggatctgggcccaacctagggctcagtgaaatctcttaatctctcattttatggagctgagacctcctacaagctagaagaatgattgccaatctgacatccttctcaggaaaaatgcaatgtttgttctgcctgcattcctaactggaggataaattcctgggggcttgagagagggaagggaagggaacatctgatgagggcgaggtgttttagagaagttccacttgccaaggaatgaattactgttggtcatgaagcaaccctggctgactcagcagagcaacagccttgccgtaacagagaacggagctcatgcacgcacacttcgactcactgactcattcagccacggccccatgctcaggctgtgcagtgcggaaccttttcctattgttgccataacaaatttccacaagattcgtgggtgaaaacaaaacggttttttaattatcttacagtgctgtagctcaaagtaggaagtgcatcttactgggctaaaatcaaggtgacagcaaggctgccttccctctgaggattccaggcaagaatctgcttctcacttgtcccagcttctaaaggctcccagttccttggctcctggtccccttcctccttcctcaaaacccacaaagactggtcacatctcacatggcatcactcagtgccttcttccttaccacacctctttctctgaatgctgctctcccttcttccttatcttttgaaaacttggggattctattgggttcaccaagatgaaaatccctcataatctcctggaaatcatccaggatacccttgttttaagttcagctgattagcaaccgtaattccatctacaatcttcattcctcctttccatgtaaaataacatattcacaaggtatggaggctaggacagggacattttggggtgggacagcattctcctgccttccacaaacagtgaacaagatgcatttggcctctgcccttgggacactgatattgcagatggttaaatgggagggcagaaaatgaatgcacaagtggatctataaatgaatgatccattgggaagcatctgtgcatgaaatctattttttgtttgttcttttgtttattgagacagagttgccctctgtcttccaggctacagtgcagtgtcacgatcttggctcactgcaacctgcttctcctggattcaagtgattctcctgcctccgcctctcgagtagctgggattacaggcaactgccaccgtgcccggctaattctttttgtatattttttgtagagaggatgtttcaccacgttggccaagcttgtctgaaactcccaacctcaagtgatccgaccgtctcagcatgccaaagtaatgggactacaggcgtgagccactgtgcccagccagaattcaaaatcaataatagataatgctgagtgtatgatttcaggtgacaaagaaggtctcactattcagatatttgtgacattaatgaaaaacacggattgaacccctgaaagattggcggaaggattttgcacacacagctgtcagccgtgaaggcacaaaggtgaaaacaatctgatgtggaaggaagaggctcttcctcaaatgctgggaatgatgtggggagaatgacaagatgactgtggagagacggagagcacactgggtacacaggaaactaaggaggaacaaggagtgtgtgtttgacactcacagccattggattcacctcggggtagccaggaatccctacatgattaatatgactgacatgaaaataagggaggctcagttgcataactggaatctaggagaccgtggaaaaggcaattgccgccccactggtgaaatgtggtgctgatttagacactaaatgaatgaagtagatggatataagataggtttgtgaggtagaatcattgactggaaaggcttgctgggtttgattttcctacttgtttaatcctcgcttaattaatttctttctgagatttattcatcctacacataaatcaatacctggcaaaggagtgacagatatatgaggggtggtggaaatgaagagacctattatagcataatatacaagtctgtgaacggtggctcacgcctgtaacccagcactgcaggaggccaaggcgggtggatcacatgaagtcagcagttcgagaccagcctggccaacatggtgaaaccctgtctctaggaaaaacacaaaaattagccgagcatggtggtgcatccctgtaatcccagctcctactctggaggatgaagcaggagaatgacttcaacccaggaggtggaggttgcagtgagtggaggttgcatcactgcactccagcctgggtggcacaaggagactccgtctcaaaaaataaaaataagaaatgcataaatataaatataatataacacacgcaaatgacaaagggacctgaattccaatcatgatttttctatttctctataattacttctttgatcctttatcttatccattaggcaatgagcctaaaacctcttccctatttggctttctgtgagcatgagatcatatagaaaatgtgaaagtccgctgaatcctccagcacagatcctggaatagagaaagtgctctggtcatcacaaaaaaaacttgcccactcacccaaatcccccacctcacccctacttccaatcacctgtggagattcaggtagaccatggggaggtaaacattaacactccttggagtgagtccagatcttggaatcagagatcagcgacagcactagctcctgctcccctttcctactaattcacaggaggacaggtggtattgaagcaatagatggccgagggggtggtccttcccccagcctctcgggtagaacagcagcctaatatgtgtctcccgagatcacaaagagcagcaggtttcacacgggcttcaacactatttcctggccgtttgacataagagaattctatttcgctttttttatcttgatttcacttttgttttctttccttggagaatgcaagttgtttgattcaagaatgctgtggatgtagaaaccctaaagcacattcgctgtgaatcaatcccagtccagtcttcccagagaagactctaaacacctcctggactgcacctgggcctatgccaattcctatcactcaccgtcactccagggagacagaacacacagagaatacgttacataggcaggttcattactaacagataagcagcgagtgacaacagaaacctatatttcaatgtgagccagtccctcaaggctcagaaaagctcctcgggacatatggagtcaccccatttgcagtgtagctgcgggaagccagaaagcagcccagcctgggttttgtaccctggagccacaggaagcactcagctaaagcactgcatgacgtcctccaggaagaacaggaagacagcccagggtgttctgagacgttcctcctgatctcaggaagttgctgtcttaggccatttttgttgctctaaaggaacacttgagcctcggtaacttctaaagaaaagagattggtttgcctcaccgttctgcaggctgtactggaagcatggcaccagcatctatttctcgtgacggcctcaggctgctcccactctggcagaagggaaggagggtctgtctgtgcagagaccacagagatcacacggcaagagagggagcaagggggagggggagtgatggagcttccaagctctttttaacaaccagctctccgggaactaatagagggggaacttgctaaccccgtctccttgggacagcattgatgtgttcatgatggatccacctccatgacccaaacacctctcaagaggcccaacctcccacagtgggggtgaaatttcaatgtgaggtttgaaggggtcaaacatctcaactaaagtagtcgtatcctcagcacgttctatggttactatgagagctataactgaaaaagcaggagaaagctgggtctcctgccatctgggtgcttgtcctaaagagatgttttatgtggttacctgtcaatcaagaaatgcgagacaattcataaagaggaactgctaagattagcttcttattggtgtctcatcttcttccaggtaacccccgacacctgcacattctgattgggacctcagtggtcatcatcctcttcatcctcctcttctttctccttcatcgctggtgctccaacaaaaaaagtaagtctcacgaagcagaggccagagagctcagggccatgtggggaagcaggatgggagcactcaggtgtgtgttcctcacaaacaggatggtccctggcccaaggcagcagccacagaggcaggactttctagagagggcaccagactccctgcccctgccttcaactcacagaccgttgcctgattctgaactgtatcctcatgtcccctgcagccactcacatccaggagaaggttccatgacaggcagaaagtgggagacagaatcaatgggatgggaactcagagctattcatgggatgggtccttgagctcagagagatagaatgtctgagtctgctgttggcaactgagggacctcagccacctatggtctccccctgtatgttggtatctgcttatgaaatgaggacccagaagtgccctccgagctgttttgttgacttccgtctcctacagatgctgcggtaatggaccaagagtctgcagggaacagaacagcgaatagcgaggtaggtactcctcggcccgggctcgtggctactgttattcccaaagagtcctggaaaatgtgagcaccctccctcactcagcatttccctctctccaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 4 - DNA, 1587 bp, Homo sapiens
cgcggccgcctgtctgcacagacagcaccatgtcgctcatggtcgtcagcatggcgtgtgttgggttcttcttgctgcagggggcctggccacatgagggagtccacagaaaaccttccctcctggcccacccaggtcgcctggtgaaatcagaagagacagtcatcctgcaatgttggtcagatgtcaggtttgagcacttccttctgcacagagaagggaagtttaaggacactttgcacctcattggagagcaccatgatggggtctccaaagccaacttctccatcggtcccatgatgcaagaccttgcagggacctacagatgctacggttctgttactcactccccctatcagttgtcagctcccagtgaccctctggacatcgtcatcacaggtctatatgagaaaccttctctctcagcccagccgggccccacggttctggcaggagagagcgtgaccttgtcctgcagctcccggagctcctatgacatgtaccatctatccagggagggggaggcccatgaatgtaggttctctgcagggcccaaggtcaacggaacattccaggccgactttcctctgggccctgccacccacggaggaacctacagatgcttcggctctttccgtgactctccatacgagtggtcaaactcgagtgacccactgcttgtttctgtcacaggaaacccttcaaatagttggccttcacccactgaaccaagctctaaaaccggtaacccccgacacctgcacattctgattgggacctcagtggtcatcatcctcttcatcctcctcttctttctccttcatcgctggtgctccaacaaaaaaaatgctgcggtaatggaccaagagtctgcagggaacagaacagcgaatagcgaggactctgatgaacaagaccctcaggaggtgacatacacacagttgaatcactgcgttttcacacagagaaaaatcactcgcccttctcagaggcccaagacacccccaacagatatcatcgtgtacacggaacttccaaatgctgagtccagatccaaagttgtctcctgcccatgagcaccacagtcaggccttgagggcgtcttctagggagacaacagccctgtctcaaaaccgggttgccagctcccatgtaccagcagctggaatctgaaggcatgagtctgcatcttagggcatcgctcttcctcacaccacaaatctgaatgtgcctctcacttgcttacaaatgtctaaggtccccactgcctgctggagaaaaaacacactcctttgcttagcccacagttctccatttcacttgacccctgcccacctctccaacctaactggcttacttcctagtctacttgaggctgcaatcacactgaggaactcacaattccaaacatacaagaggctccctcttaacgcagcacttagacacgtgttgttccaccttccctcatgctgttccacctcccctcagactagctttcagtcttctgtcagcagtaaaacttatatattttttaaaataacttcaatgtagttttccatccttcaaataaacatgtctgcccccatg

SEQ ID NO: 5 - DNA, 20 bp, Artificial Sequence
  Synthetic Construct
ctcttgagcgagcacccact

SEQ ID NO: 6 - DNA, 20 bp, Artificial Sequence
  Synthetic Construct
gctcttgagcgagcacccac

SEQ ID NO: 7 - DNA, 20 bp, Artificial Sequence
  Synthetic Construct
agtgggtgctcgctcaagag

SEQ ID NO: 8 - DNA, 20 bp, Artificial Sequence
  Synthetic Construct
gctcgctcaagagcggaaca

SEQ ID NO: 9 - DNA, 20 bp, Artificial Sequence
  Synthetic Construct
taccttgcatgaggcccagt

SEQ ID NO: 10 - DNA, 20 bp, Artificial Sequence
  Synthetic Construct
tggtcaacatgtaaactgca

SEQ ID NO: 11 - DNA, 20 bp, Artificial Sequence
  Synthetic Construct
tgtgcgctgctgagctgagc

SEQ ID NO: 12 - DNA, 20 bp, Artificial Sequence
  Synthetic Construct
ggtcaacatgtaaactgcat

SEQ ID NO: 13 - DNA, 20 bp, Artificial Sequence
  Synthetic Construct
catgggagggtgacgtacgc

SEQ ID NO: 14 - DNA, 20 bp, Artificial Sequence
  Synthetic Construct
atgttgaccacatcatggga

SEQ ID NO: 15 - DNA, 20 bp, Artificial Sequence
  Synthetic Construct
tttacatgttgaccacatca

SEQ ID NO: 16 - DNA, 20 bp, Artificial Sequence
  Synthetic Construct
acatgtaaactgcatgggca

SEQ ID NO: 17 - DNA, 20 bp, Artificial Sequence
  Synthetic Construct
gcagcgcacaggatgttatt

SEQ ID NO: 18 - DNA, 20 bp, Artificial Sequence
  Synthetic Construct
tgcgctgctgagctgagctg

SEQ ID NO: 19 - DNA, 20 bp, Artificial Sequence
  Synthetic Construct
aacatgtaaactgcatgggc

SEQ ID NO: 20 - DNA, 20 bp, Artificial Sequence
  Synthetic Construct
agctcagctcagcagcgcac

SEQ ID NO: 21 - DNA, 20 bp, Artificial Sequence
  Synthetic Construct
ttacatgttgaccacatcat

SEQ ID NO: 22 - DNA, 20 bp, Artificial Sequence
  Synthetic Construct
gtgcgctgctgagctgagct

SEQ ID NO: 23 - DNA, 20 bp, Artificial Sequence
  Synthetic Construct
tacgtcaccctcccatgatg

SEQ ID NO: 24 - DNA, 20 bp, Artificial Sequence
  Synthetic Construct
catgttgaccacatcatggg

SEQ ID NO: 25 - DNA, 20 bp, Artificial Sequence
  Synthetic Construct
ggctctttctaccttgcatg

SEQ ID NO: 26 - DNA, 20 bp, Artificial Sequence
  Synthetic Construct
ctaccttgcatgaggcccag

SEQ ID NO: 27 - DNA, 20 bp, Artificial Sequence
  Synthetic Construct
cacccactgggcctcatgca

SEQ ID NO: 28 - DNA, 20 bp, Artificial Sequence
  Synthetic Construct
agacagcaccatgtcgctca

SEQ ID NO: 29 - DNA, 20 bp, Artificial Sequence
  Synthetic Construct
atggtgctgtctgtgcagac

SEQ ID NO: 30 - DNA, 20 bp, Artificial Sequence
  Synthetic Construct
gtgctgtctgtgcagacagg

SEQ ID NO: 31 - DNA, 20 bp, Artificial Sequence
  Synthetic Construct
gctgacgaccatgagcgaca

SEQ ID NO: 32 - DNA, 20 bp, Artificial Sequence
  Synthetic Construct
gtcgctcatggtcgtcagca

SEQ ID NO: 33 - DNA, 20 bp, Artificial Sequence
  Synthetic Construct
cagggggcctggccacatga

SEQ ID NO: 34 - DNA, 20 bp, Artificial Sequence
  Synthetic Construct
ggactcaccctcatgtggcc

SEQ ID NO: 35 - DNA, 20 bp, Artificial Sequence
  Synthetic Construct
cagggttcttcttgctgcag

SEQ ID NO: 36 - DNA, 20 bp, Artificial Sequence
  Synthetic Construct
ccagggttcttcttgctgca

SEQ ID NO: 37 - DNA, 20 bp, Artificial Sequence
  Synthetic Construct
tccagggttcttcttgctgc

SEQ ID NO: 38 - DNA, 20 bp, Artificial Sequence
  Synthetic Construct
tcttcttgctgcagggggcc

SEQ ID NO: 39 - DNA, 20 bp, Artificial Sequence
  Synthetic Construct
agggttcttcttgctgcagg

SEQ ID NO: 40 - DNA, 20 bp, Artificial Sequence
  Synthetic Construct
gcagggggcctggccacatg

SEQ ID NO: 41 - DNA, 20 bp, Artificial Sequence
  Synthetic Construct
ccctgcagcaagaagaaccc

SEQ ID NO: 42 - DNA, 20 bp, Artificial Sequence
  Synthetic Construct
cctgcaaggtcttgcatcat

SEQ ID NO: 43 - DNA, 20 bp, Artificial Sequence
  Synthetic Construct
cccatgatgcaagaccttgc

SEQ ID NO: 44 - DNA, 20 bp, Artificial Sequence
  Synthetic Construct
ccctgcaaggtcttgcatca

SEQ ID NO: 45 - DNA, 20 bp, Artificial Sequence
  Synthetic Construct
ccatgatgcaagaccttgca

SEQ ID NO: 46 - DNA, 20 bp, Artificial Sequence
  Synthetic Construct
aaggacactttgcacctcat

SEQ ID NO: 47 - DNA, 20 bp, Artificial Sequence
  Synthetic Construct
gtcttgcatcatgggaccga

SEQ ID NO: 48 - DNA, 20 bp, Artificial Sequence
  Synthetic Construct
gcacagagaagggaagttta

SEQ ID NO: 49 - DNA, 20 bp, Artificial Sequence
  Synthetic Construct
accgatggagaagttggctt

SEQ ID NO: 50 - DNA, 20 bp, Artificial Sequence
  Synthetic Construct
tctgatttcaccaggcgacc

SEQ ID NO: 51 - DNA, 20 bp, Artificial Sequence
  Synthetic Construct
ctgtgatgacgatgtccaga

SEQ ID NO: 52 - DNA, 20 bp, Artificial Sequence
  Synthetic Construct
catcatggtgctctccaatg

SEQ ID NO: 53 - DNA, 20 bp, Artificial Sequence
  Synthetic Construct
cctggcccacccaggtcgcc

SEQ ID NO: 54 - DNA, 20 bp, Artificial Sequence
  Synthetic Construct
tgcaatgttggtcagatgtc

SEQ ID NO: 55 - DNA, 20 bp, Artificial Sequence
  Synthetic Construct
ctgatttcaccaggcgacct

SEQ ID NO: 56 - DNA, 20 bp, Artificial Sequence
  Synthetic Construct
tcattggagagcaccatgat

SEQ ID NO: 57 - DNA, 20 bp, Artificial Sequence
  Synthetic Construct
ctcattggagagcaccatga

SEQ ID NO: 58 - DNA, 20 bp, Artificial Sequence
  Synthetic Construct
cattggagagcaccatgatg

SEQ ID NO: 59 - DNA, 20 bp, Artificial Sequence
  Synthetic Construct
tttcaccaggcgacctgggt

SEQ ID NO: 60 - DNA, 20 bp, Artificial Sequence
  Synthetic Construct
atttcaccaggcgacctggg

SEQ ID NO: 61 - DNA, 20 bp, Artificial Sequence
  Synthetic Construct
tccaaagccaacttctccat

SEQ ID NO: 62 - DNA, 20 bp, Artificial Sequence
  Synthetic Construct
acttccttctgcacagagaa

SEQ ID NO: 63 - DNA, 20 bp, Artificial Sequence
  Synthetic Construct
acttcccttctctgtgcaga

SEQ ID NO: 64 - DNA, 20 bp, Artificial Sequence
  Synthetic Construct
ccaggcgacctgggtgggcc

SEQ ID NO: 65 - DNA, 20 bp, Artificial Sequence
  Synthetic Construct
gacgatgtccagagggtcac

SEQ ID NO: 66 - DNA, 20 bp, Artificial Sequence
  Synthetic Construct
tggctttggagaccccatca

SEQ ID NO: 67 - DNA, 20 bp, Artificial Sequence
  Synthetic Construct
agcatctgtaggtccctgca

SEQ ID NO: 68 - DNA, 20 bp, Artificial Sequence
  Synthetic Construct
catgggaccgatggagaagt

SEQ ID NO: 69 - DNA, 20 bp, Artificial Sequence
  Synthetic Construct
gggagctgacaactgatagg

SEQ ID NO: 70 - DNA, 20 bp, Artificial Sequence
  Synthetic Construct
ctgtctcttctgatttcacc

SEQ ID NO: 71 - DNA, 20 bp, Artificial Sequence
  Synthetic Construct
ctgggagctgacaactgata

SEQ ID NO: 72 - DNA, 20 bp, Artificial Sequence
  Synthetic Construct
tgggagctgacaactgatag

SEQ ID NO: 73 - DNA, 20 bp, Artificial Sequence
  Synthetic Construct
aacagaaccgtagcatctgt

SEQ ID NO: 74 - DNA, 20 bp, Artificial Sequence
  Synthetic Construct
actgggagctgacaactgat

SEQ ID NO: 75 - DNA, 20 bp, Artificial Sequence
  Synthetic Construct
gtcagctcccagtgaccctc

SEQ ID NO: 76 - DNA, 20 bp, Artificial Sequence
  Synthetic Construct
acgatgtccagagggtcact

SEQ ID NO: 77 - DNA, 20 bp, Artificial Sequence
  Synthetic Construct
ggcgacctgggtgggccagg

SEQ ID NO: 78 - DNA, 20 bp, Artificial Sequence
  Synthetic Construct
gcgacctgggtgggccagga

SEQ ID NO: 79 - DNA, 20 bp, Artificial Sequence
  Synthetic Construct
tgacatctgaccaacattgc

SEQ ID NO: 80 - DNA, 20 bp, Artificial Sequence
  Synthetic Construct
gcagggacctacagatgcta

SEQ ID NO: 81 - DNA, 20 bp, Artificial Sequence
  Synthetic Construct
agacagtcatcctgcaatgt

SEQ ID NO: 82 - DNA, 20 bp, Artificial Sequence
  Synthetic Construct
cacttccttctgcacagaga

SEQ ID NO: 83 - DNA, 20 bp, Artificial Sequence
  Synthetic Construct
ccacagaaaaccttccctcc

SEQ ID NO: 84 - DNA, 20 bp, Artificial Sequence
  Synthetic Construct
ccttccctcctggcccaccc

SEQ ID NO: 85 - DNA, 20 bp, Artificial Sequence
  Synthetic Construct
ccaggagggaaggttttctg

SEQ ID NO: 86 - DNA, 20 bp, Artificial Sequence
  Synthetic Construct
cctgggtgggccaggaggga

SEQ ID NO: 87 - DNA, 20 bp, Artificial Sequence
  Synthetic Construct
ctgcagagaacctacattca

SEQ ID NO: 88 - DNA, 20 bp, Artificial Sequence
  Synthetic Construct
agggggaggcccatgaatgt

SEQ ID NO: 89 - DNA, 20 bp, Artificial Sequence
  Synthetic Construct
tgcagagaacctacattcat

SEQ ID NO: 90 - DNA, 20 bp, Artificial Sequence
  Synthetic Construct
catgaatgtaggttctctgc

SEQ ID NO: 91 - DNA, 20 bp, Artificial Sequence
  Synthetic Construct
atgaatgtaggttctctgca

SEQ ID NO: 92 - DNA, 20 bp, Artificial Sequence
  Synthetic Construct
tccgtgactctccatacgag

SEQ ID NO: 93 - DNA, 20 bp, Artificial Sequence
  Synthetic Construct
gctctctcctgccagaaccg

SEQ ID NO: 94 - DNA, 20 bp, Artificial Sequence
  Synthetic Construct
agcatctgtaggttcctccg

SEQ ID NO: 95 - DNA, 20 bp, Artificial Sequence
  Synthetic Construct
taggttctctgcagggccca

SEQ ID NO: 96 - DNA, 20 bp, Artificial Sequence
  Synthetic Construct
ctcgagtttgaccactcgta

SEQ ID NO: 97 - DNA, 20 bp, Artificial Sequence
  Synthetic Construct
accactcgtatggagagtca

SEQ ID NO: 98 - DNA, 20 bp, Artificial Sequence
  Synthetic Construct
ctctctcctgccagaaccgt

SEQ ID NO: 99 - DNA, 20 bp, Artificial Sequence
  Synthetic Construct
ggcccagaggaaagtcggcc

SEQ ID NO: 100 - DNA, 20 bp, Artificial Sequence
  Synthetic Construct
ctggaatgttccgttgacct

SEQ ID NO: 101 - DNA, 20 bp, Artificial Sequence
  Synthetic Construct
tggaatgttccgttgacctt

SEQ ID NO: 102 - DNA, 20 bp, Artificial Sequence
  Synthetic Construct
tctgcagggcccaaggtcaa

SEQ ID NO: 103 - DNA, 20 bp, Artificial Sequence
  Synthetic Construct
gcatctgtaggttcctccgt

SEQ ID NO: 104 - DNA, 20 bp, Artificial Sequence
  Synthetic Construct
ggatagatggtacatgtcat

SEQ ID NO: 105 - DNA, 20 bp, Artificial Sequence
  Synthetic Construct
caaggtcaacggaacattcc

SEQ ID NO: 106 - DNA, 20 bp, Artificial Sequence
  Synthetic Construct
ccgggccccacggttctggc

SEQ ID NO: 107 - DNA, 20 bp, Artificial Sequence
  Synthetic Construct
atgacatgtaccatctatcc

SEQ ID NO: 108 - DNA, 20 bp, Artificial Sequence
  Synthetic Construct
tctctcctgccagaaccgtg

SEQ ID NO: 109 - DNA, 20 bp, Artificial Sequence
  Synthetic Construct
ttcatgggcctccccctccc

SEQ ID NO: 110 - DNA, 20 bp, Artificial Sequence
  Synthetic Construct
tgtaccatctatccagggag

SEQ ID NO: 111 - DNA, 20 bp, Artificial Sequence
  Synthetic Construct
ataggagctccgggagctgc

SEQ ID NO: 112 - DNA, 20 bp, Artificial Sequence
  Synthetic Construct
attccaggccgactttcctc

SEQ ID NO: 113 - DNA, 20 bp, Artificial Sequence
  Synthetic Construct
taggttcctccgtgggtggc

SEQ ID NO: 114 - DNA, 20 bp, Artificial Sequence
  Synthetic Construct
ttccaggccgactttcctct

SEQ ID NO: 115 - DNA, 20 bp, Artificial Sequence
  Synthetic Construct
atgtaccatctatccaggga

SEQ ID NO: 116 - DNA, 20 bp, Artificial Sequence
  Synthetic Construct
tgacatgtaccatctatcca

SEQ ID NO: 117 - DNA, 20 bp, Artificial Sequence
  Synthetic Construct
ccagaaccgtggggcccggc

SEQ ID NO: 118 - DNA, 20 bp, Artificial Sequence
  Synthetic Construct
cctgccagaaccgtggggcc

SEQ ID NO: 119 - DNA, 20 bp, Artificial Sequence
  Synthetic Construct
cagaaccgtggggcccggct

SEQ ID NO: 120 - DNA, 20 bp, Artificial Sequence
  Synthetic Construct
ggaggaacctacagatgctt

SEQ ID NO: 121 - DNA, 20 bp, Artificial Sequence
  Synthetic Construct
ccagccgggccccacggttc

SEQ ID NO: 122 - DNA, 20 bp, Artificial Sequence
  Synthetic Construct
gtaccatctatccagggagg

SEQ ID NO: 123 - DNA, 20 bp, Artificial Sequence
  Synthetic Construct
gtacatgtcataggagctcc

SEQ ID NO: 124 - DNA, 20 bp, Artificial Sequence
  Synthetic Construct
catgtaccatctatccaggg

SEQ ID NO: 125 - DNA, 20 bp, Artificial Sequence
  Synthetic Construct
ccatctatccagggaggggg

SEQ ID NO: 126 - DNA, 20 bp, Artificial Sequence
  Synthetic Construct
ggtacatgtcataggagctc

SEQ ID NO: 127 - DNA, 20 bp, Artificial Sequence
  Synthetic Construct
ggcagggcccagaggaaagt

SEQ ID NO: 128 - DNA, 20 bp, Artificial Sequence
  Synthetic Construct
cctccccctccctggataga

SEQ ID NO: 129 - DNA, 20 bp, Artificial Sequence
  Synthetic Construct
tctgtaggttcctccgtggg

SEQ ID NO: 130 - DNA, 20 bp, Artificial Sequence
  Synthetic Construct
ctgtgacagaaacaagcagt

SEQ ID NO: 131 - DNA, 20 bp, Artificial Sequence
  Synthetic Construct
gctccgggagctgcaggaca

SEQ ID NO: 132 - DNA, 20 bp, Artificial Sequence
  Synthetic Construct
tgaccttgtcctgcagctcc

SEQ ID NO: 133 - DNA, 20 bp, Artificial Sequence
  Synthetic Construct
aggttcctccgtgggtggca

SEQ ID NO: 134 - DNA, 20 bp, Artificial Sequence
  Synthetic Construct
ctcagcccagccgggcccca

SEQ ID NO: 135 - DNA, 20 bp, Artificial Sequence
  Synthetic Construct
ctgggccctgccacccacgg

SEQ ID NO: 136 - DNA, 20 bp, Artificial Sequence
  Synthetic Construct
ccttctctctcagcccagcc

SEQ ID NO: 137 - DNA, 20 bp, Artificial Sequence
  Synthetic Construct
gaaagagccgaagcatctgt

SEQ ID NO: 138 - DNA, 20 bp, Artificial Sequence
  Synthetic Construct
accttctctctcagcccagc

SEQ ID NO: 139 - DNA, 20 bp, Artificial Sequence
  Synthetic Construct
cccggctgggctgagagaga

SEQ ID NO: 140 - DNA, 20 bp, Artificial Sequence
  Synthetic Construct
ccgtgggtggcagggcccag

SEQ ID NO: 141 - DNA, 20 bp, Artificial Sequence
  Synthetic Construct
cctctgggccctgccaccca

SEQ ID NO: 142 - DNA, 20 bp, Artificial Sequence
  Synthetic Construct
tactcaccggttttagagct

SEQ ID NO: 143 - DNA, 20 bp, Artificial Sequence
  Synthetic Construct
actgaaccaagctctaaaac

SEQ ID NO: 144 - DNA, 20 bp, Artificial Sequence
  Synthetic Construct
gttttagagcttggttcagt

SEQ ID NO: 145 - DNA, 20 bp, Artificial Sequence
  Synthetic Construct
gttttagagcttggttcagt

SEQ ID NO: 146 - DNA, 20 bp, Artificial Sequence
  Synthetic Construct
ggttttagagcttggttcag

SEQ ID NO: 147 - DNA, 20 bp, Artificial Sequence
  Synthetic Construct
taggaaacccttcaaatagt

SEQ ID NO: 148 - DNA, 20 bp, Artificial Sequence
  Synthetic Construct
taggaaacccttcaaatagt

SEQ ID NO: 149 - DNA, 20 bp, Artificial Sequence
  Synthetic Construct
gagcttggttcagtgggtga

SEQ ID NO: 150 - DNA, 20 bp, Artificial Sequence
  Synthetic Construct
tcagaatgtgcaggtgtcgg

SEQ ID NO: 151 - DNA, 20 bp, Artificial Sequence
  Synthetic Construct
tcagaatgtgcaggtgtcgg

SEQ ID NO: 152 - DNA, 20 bp, Artificial Sequence
  Synthetic Construct
cgacacctgcacattctgat

SEQ ID NO: 153 - DNA, 20 bp, Artificial Sequence
  Synthetic Construct
tgcaggtgtcgggggttacc

SEQ ID NO: 154 - DNA, 20 bp, Artificial Sequence
  Synthetic Construct
tgcaggtgtcgggggttacc

SEQ ID NO: 155 - DNA, 20 bp, Artificial Sequence
  Synthetic Construct
aatcagaatgtgcaggtgtc

SEQ ID NO: 156 - DNA, 20 bp, Artificial Sequence
  Synthetic Construct
aggtcccaatcagaatgtgc

SEQ ID NO: 157 - DNA, 20 bp, Artificial Sequence
  Synthetic Construct
gacacctgcacattctgatt

SEQ ID NO: 158 - DNA, 20 bp, Artificial Sequence
  Synthetic Construct
gacacctgcacattctgatt

SEQ ID NO: 159 - DNA, 20 bp, Artificial Sequence
  Synthetic Construct
cattctgattgggacctcag

SEQ ID NO: 160 - DNA, 20 bp, Artificial Sequence
  Synthetic Construct
cattctgattgggacctcag

SEQ ID NO: 161 - DNA, 20 bp, Artificial Sequence
  Synthetic Construct
gtgagacttactttttttgt

SEQ ID NO: 162 - DNA, 20 bp, Artificial Sequence
  Synthetic Construct
tcttctttctccttcatcgc

SEQ ID NO: 163 - DNA, 20 bp, Artificial Sequence
  Synthetic Construct
gaaagaagaggaggatgaag

SEQ ID NO: 164 - DNA, 20 bp, Artificial Sequence
  Synthetic Construct
agcgatgaaggagaaagaag

SEQ ID NO: 165 - DNA, 20 bp, Artificial Sequence
  Synthetic Construct
agcgatgaaggagaaagaag

SEQ ID NO: 166 - DNA, 20 bp, Artificial Sequence
  Synthetic Construct
tcctacagatgctgcggtaa

SEQ ID NO: 167 - DNA, 20 bp, Artificial Sequence
  Synthetic Construct
tccattaccgcagcatctgt

SEQ ID NO: 168 - DNA, 20 bp, Artificial Sequence
  Synthetic Construct
taatggaccaagagtctgca

SEQ ID NO: 169 - DNA, 20 bp, Artificial Sequence
  Synthetic Construct
gaacagaacagcgaatagcg

SEQ ID NO: 170 - DNA, 20 bp, Artificial Sequence
  Synthetic Construct
gaacagaacagcgaatagcg

SEQ ID NO: 171 - DNA, 20 bp, Artificial Sequence
  Synthetic Construct
gtaatggaccaagagtctgc

SEQ ID NO: 172 - DNA, 20 bp, Artificial Sequence
  Synthetic Construct
gtaatggaccaagagtctgc

SEQ ID NO: 173 - DNA, 20 bp, Artificial Sequence
  Synthetic Construct
aacagatatcatcgtgtaca

SEQ ID NO: 174 - DNA, 20 bp, Artificial Sequence
  Synthetic Construct
gtacacgatgatatctgttg

SEQ ID NO: 175 - DNA, 20 bp, Artificial Sequence
  Synthetic Construct
gtgtacacgatgatatctgt

SEQ ID NO: 176 - DNA, 20 bp, Artificial Sequence
  Synthetic Construct
tgtacacgatgatatctgtt

SEQ ID NO: 177 - DNA, 20 bp, Artificial Sequence
  Synthetic Construct
tacacgatgatatctgttgg

SEQ ID NO: 178 - DNA, 20 bp, Artificial Sequence
  Synthetic Construct
ttggatctggactcagcatt

SEQ ID NO: 179 - DNA, 20 bp, Artificial Sequence
  Synthetic Construct
tgtgtgtatgtcacctcctg

SEQ ID NO: 180 - DNA, 20 bp, Artificial Sequence
  Synthetic Construct
gtgtgtatgtcacctcctga

SEQ ID NO: 181 - DNA, 20 bp, Artificial Sequence
  Synthetic Construct
gcaggagacaactttggatc

SEQ ID NO: 182 - DNA, 20 bp, Artificial Sequence
  Synthetic Construct
tcatgggcaggagacaactt

SEQ ID NO: 183 - DNA, 20 bp, Artificial Sequence
  Synthetic Construct
atatctgttgggggtgtctt

SEQ ID NO: 184 - DNA, 20 bp, Artificial Sequence
  Synthetic Construct
gatatctgttgggggtgtct

SEQ ID NO: 185 - DNA, 20 bp, Artificial Sequence
  Synthetic Construct
ggtcttgttcatcagagtcc

SEQ ID NO: 186 - DNA, 20 bp, Artificial Sequence
  Synthetic Construct
ctctgatgaacaagaccctc

SEQ ID NO: 187 - DNA, 20 bp, Artificial Sequence
  Synthetic Construct
aaatcactcgcccttctcag

SEQ ID NO: 188 - DNA, 20 bp, Artificial Sequence
  Synthetic Construct
gtgtcttgggcctctgagaa

SEQ ID NO: 189 - DNA, 20 bp, Artificial Sequence
  Synthetic Construct
tgatgaacaagaccctcagg

SEQ ID NO: 190 - DNA, 20 bp, Artificial Sequence
  Synthetic Construct
ggtgtcttgggcctctgaga

SEQ ID NO: 191 - DNA, 20 bp, Artificial Sequence
  Synthetic Construct
gtctaagtgctgcgttaaga

SEQ ID NO: 192 - DNA, 20 bp, Artificial Sequence
  Synthetic Construct
tgtctaagtgctgcgttaag

SEQ ID NO: 193 - DNA, 20 bp, Artificial Sequence
  Synthetic Construct
gagggagcctcttgtatgtt

SEQ ID NO: 194 - DNA, 20 bp, Artificial Sequence
  Synthetic Construct
gcccacctctccaacctaac

SEQ ID NO: 195 - DNA, 20 bp, Artificial Sequence
  Synthetic Construct
attgcagcctcaagtagact

SEQ ID NO: 196 - DNA, 20 bp, Artificial Sequence
  Synthetic Construct
gactaggaagtaagccagtt

SEQ ID NO: 197 - DNA, 20 bp, Artificial Sequence
  Synthetic Construct
aggaagtaagccagttaggt

SEQ ID NO: 198 - DNA, 20 bp, Artificial Sequence
  Synthetic Construct
gtaagccagttaggttggag

SEQ ID NO: 199 - DNA, 20 bp, Artificial Sequence
  Synthetic Construct
agactgaaagctagtctgag

SEQ ID NO: 200 - DNA, 20 bp, Artificial Sequence
  Synthetic Construct
cttacttcctagtctacttg

SEQ ID NO: 201 - DNA, 20 bp, Artificial Sequence
  Synthetic Construct
gaagactgaaagctagtctg

SEQ ID NO: 202 - DNA, 20 bp, Artificial Sequence
  Synthetic Construct
ctccctagaagacgccctca

SEQ ID NO: 203 - DNA, 20 bp, Artificial Sequence
  Synthetic Construct
ctgaaagctagtctgagggg

SEQ ID NO: 204 - DNA, 20 bp, Artificial Sequence
  Synthetic Construct
ttgaggctgcaatcacactg

SEQ ID NO: 205 - DNA, 20 bp, Artificial Sequence
  Synthetic Construct
aagactgaaagctagtctga

SEQ ID NO: 206 - DNA, 20 bp, Artificial Sequence
  Synthetic Construct
gccagttaggttggagaggt

SEQ ID NO: 207 - DNA, 20 bp, Artificial Sequence
  Synthetic Construct
aggccttgagggcgtcttct

SEQ ID NO: 208 - DNA, 20 bp, Artificial Sequence
  Synthetic Construct
tgggcaggggtcaagtgaaa

SEQ ID NO: 209 - DNA, 20 bp, Artificial Sequence
  Synthetic Construct
ggccttgagggcgtcttcta

SEQ ID NO: 210 - DNA, 20 bp, Artificial Sequence
  Synthetic Construct
cacaattccaaacatacaag

SEQ ID NO: 211 - DNA, 20 bp, Artificial Sequence
  Synthetic Construct
ggggaggtggaacagcatga

SEQ ID NO: 212 - DNA, 20 bp, Artificial Sequence
  Synthetic Construct
aggggaggtggaacagcatg

SEQ ID NO: 213 - DNA, 20 bp, Artificial Sequence
  Synthetic Construct
agccagttaggttggagagg

SEQ ID NO: 214 - DNA, 20 bp, Artificial Sequence
  Synthetic Construct
aaagctagtctgaggggagg

SEQ ID NO: 215 - DNA, 20 bp, Artificial Sequence
  Synthetic Construct
aggtggaacagcatgaggga

SEQ ID NO: 216 - DNA, 20 bp, Artificial Sequence
  Synthetic Construct
tcaagtgaaatggagaactg

SEQ ID NO: 217 - DNA, 20 bp, Artificial Sequence
  Synthetic Construct
caagtgaaatggagaactgt

SEQ ID NO: 218 - DNA, 20 bp, Artificial Sequence
  Synthetic Construct
gttaggttggagaggtgggc

SEQ ID NO: 219 - DNA, 20 bp, Artificial Sequence
  Synthetic Construct
tggaacagcatgagggaagg

SEQ ID NO: 220 - DNA, 20 bp, Artificial Sequence
  Synthetic Construct
acgccctcaaggcctgactg

SEQ ID NO: 221 - DNA, 20 bp, Artificial Sequence
  Synthetic Construct
gggcagacatgtttatttga

SEQ ID NO: 222 - DNA, 20 bp, Artificial Sequence
  Synthetic Construct
taggttggagaggtgggcag

SEQ ID NO: 223 - DNA, 20 bp, Artificial Sequence
  Synthetic Construct
agacatgtttatttgaagga

SEQ ID NO: 224 - DNA, 20 bp, Artificial Sequence
  Synthetic Construct
gagtgtgttttttctccagc

SEQ ID NO: 225 - DNA, 20 bp, Artificial Sequence
  Synthetic Construct
ttaggttggagaggtgggca

SEQ ID NO: 226 - DNA, 20 bp, Artificial Sequence
  Synthetic Construct
gacatttgtaagcaagtgag

SEQ ID NO: 227 - DNA, 20 bp, Artificial Sequence
  Synthetic Construct
acattcagatttgtggtgtg

SEQ ID NO: 228 - DNA, 20 bp, Artificial Sequence
  Synthetic Construct
gagaggcacattcagatttg

SEQ ID NO: 229 - DNA, 20 bp, Artificial Sequence
  Synthetic Construct
aaggcatgagtctgcatctt

SEQ ID NO: 230 - DNA, 20 bp, Artificial Sequence
  Synthetic Construct
gttttttctccagcaggcag

SEQ ID NO: 231 - DNA, 20 bp, Artificial Sequence
  Synthetic Construct
gagaactgtgggctaagcaa

SEQ ID NO: 232 - DNA, 20 bp, Artificial Sequence
  Synthetic Construct
aggcatgagtctgcatctta

SEQ ID NO: 233 - DNA, 20 bp, Artificial Sequence
  Synthetic Construct
ggcctgactgtggtgctcat

SEQ ID NO: 234 - DNA, 20 bp, Artificial Sequence
  Synthetic Construct
agattccagctgctggtaca

SEQ ID NO: 235 - DNA, 20 bp, Artificial Sequence
  Synthetic Construct
gtacatgggagctggcaacc

SEQ ID NO: 236 - DNA, 20 bp, Artificial Sequence
  Synthetic Construct
cacttgcttacaaatgtcta

SEQ ID NO: 237 - DNA, 20 bp, Artificial Sequence
  Synthetic Construct
agctgctggtacatgggagc

SEQ ID NO: 238 - DNA, 20 bp, Artificial Sequence
  Synthetic Construct
agcaccacagtcaggccttg

SEQ ID NO: 239 - DNA, 20 bp, Artificial Sequence
  Synthetic Construct
ctaaggtccccactgcctgc

SEQ ID NO: 240 - DNA, 20 bp, Artificial Sequence
  Synthetic Construct
ttttttctccagcaggcagt

SEQ ID NO: 241 - DNA, 20 bp, Artificial Sequence
  Synthetic Construct
aggcctgactgtggtgctca

SEQ ID NO: 242 - DNA, 20 bp, Artificial Sequence
  Synthetic Construct
agctcccatgtaccagcagc

SEQ ID NO: 243 - DNA, 20 bp, Artificial Sequence
  Synthetic Construct
ggcaacccggttttgagaca

SEQ ID NO: 244 - DNA, 20 bp, Artificial Sequence
  Synthetic Construct
tttttctccagcaggcagtg

SEQ ID NO: 245 - DNA, 20 bp, Artificial Sequence
  Synthetic Construct
tggcaacccggttttgagac

SEQ ID NO: 246 - DNA, 20 bp, Artificial Sequence
  Synthetic Construct
caacagccctgtctcaaaac

SEQ ID NO: 247 - DNA, 20 bp, Artificial Sequence
  Synthetic Construct
gcaccacagtcaggccttga

SEQ ID NO: 248 - DNA, 20 bp, Artificial Sequence
  Synthetic Construct
aacagccctgtctcaaaacc

SEQ ID NO: 249 - DNA, 20 bp, Artificial Sequence
  Synthetic Construct
gattccagctgctggtacat

SEQ ID NO: 250 - DNA, 20 bp, Artificial Sequence
  Synthetic Construct
tgccttcagattccagctgc

SEQ ID NO: 251 - DNA, 20 bp, Artificial Sequence
  Synthetic Construct
taccagcagctggaatctga

SEQ ID NO: 252 - DNA, 21 bp, Artificial Sequence
  Synthetic Construct
taccttgcatgaggcccagtg

SEQ ID NO: 253 - DNA, 21 bp, Artificial Sequence
  Synthetic Construct
catgttgaccacatcatggga

SEQ ID NO: 254 - DNA, 21 bp, Artificial Sequence
  Synthetic Construct
gcgccctgcccatgcagttta

SEQ ID NO: 255 - DNA, 21 bp, Artificial Sequence
  Synthetic Construct
cagggttcttcttgctgcagg

SEQ ID NO: 256 - DNA, 21 bp, Artificial Sequence
  Synthetic Construct
tttccagggttcttcttgctg

SEQ ID NO: 257 - DNA, 21 bp, Artificial Sequence
  Synthetic Construct
gagaccccatcatggtgctct

SEQ ID NO: 258 - DNA, 21 bp, Artificial Sequence
  Synthetic Construct
aggacactttgcacctcattg

SEQ ID NO: 259 - DNA, 21 bp, Artificial Sequence
  Synthetic Construct
cacctcattggagagcaccat

SEQ ID NO: 260 - DNA, 21 bp, Artificial Sequence
  Synthetic Construct
accaggcgacctgggtgggcc

SEQ ID NO: 261 - DNA, 21 bp, Artificial Sequence
  Synthetic Construct
agcacttccttctgcacagag

SEQ ID NO: 262 - DNA, 21 bp, Artificial Sequence
  Synthetic Construct
accactcgtatggagagtcac

SEQ ID NO: 263 - DNA, 21 bp, Artificial Sequence
  Synthetic Construct
cgtgactctccatacgagtgg

SEQ ID NO: 264 - DNA, 21 bp, Artificial Sequence
  Synthetic Construct
ctctgggccctgccacccacg

SEQ ID NO: 265 - DNA, 21 bp, Artificial Sequence
  Synthetic Construct
agagcttggttcagtgggtga

SEQ ID NO: 266 - DNA, 21 bp, Artificial Sequence
  Synthetic Construct
gagcttggttcagtgggtgaa

SEQ ID NO: 267 - DNA, 21 bp, Artificial Sequence
  Synthetic Construct
gttggagcaccagcgatgaag

SEQ ID NO: 268 - DNA, 21 bp, Artificial Sequence
  Synthetic Construct
ttggagcaccagcgatgaagg

SEQ ID NO: 269 - DNA, 21 bp, Artificial Sequence
  Synthetic Construct
tgttggagcaccagcgatgaa

SEQ ID NO: 270 - DNA, 21 bp, Artificial Sequence
  Synthetic Construct
tttgttggagcaccagcgatg

SEQ ID NO: 271 - DNA, 21 bp, Artificial Sequence
  Synthetic Construct
ttgttggagcaccagcgatga

SEQ ID NO: 272 - DNA, 21 bp, Artificial Sequence
  Synthetic Construct
tccttcatcgctggtgctcca

SEQ ID NO: 273 - DNA, 21 bp, Artificial Sequence
  Synthetic Construct
gaagttccgtgtacacgatga

SEQ ID NO: 274 - DNA, 21 bp, Artificial Sequence
  Synthetic Construct
gatctggactcagcatttgga

SEQ ID NO: 275 - DNA, 21 bp, Artificial Sequence
  Synthetic Construct
tctgtgtgaaaacgcagtgat

SEQ ID NO: 276 - DNA, 21 bp, Artificial Sequence
  Synthetic Construct
ctctgtgtgaaaacgcagtga

SEQ ID NO: 277 - DNA, 21 bp, Artificial Sequence
  Synthetic Construct
tctctgtgtgaaaacgcagtg

SEQ ID NO: 278 - DNA, 21 bp, Artificial Sequence
  Synthetic Construct
acacagagaaaaatcactcgc

SEQ ID NO: 279 - DNA, 21 bp, Artificial Sequence
  Synthetic Construct
cacacagagaaaaatcactcg

SEQ ID NO: 280 - DNA, 21 bp, Artificial Sequence
  Synthetic Construct
acttgacccctgcccacctct

SEQ ID NO: 281 - DNA, 21 bp, Artificial Sequence
  Synthetic Construct
ctccagcaggcagtggggacc

SEQ ID NO: 282 - DNA, 21 bp, Artificial Sequence
  Synthetic Construct
gaattgtgagttcctcagtgt

SEQ ID NO: 283 - DNA, 21 bp, Artificial Sequence
  Synthetic Construct
actgctgacagaagactgaaa

SEQ ID NO: 284 - DNA, 21 bp, Artificial Sequence
  Synthetic Construct
tccagcaggcagtggggacct

SEQ ID NO: 285 - DNA, 21 bp, Artificial Sequence
  Synthetic Construct
agtcttctgtcagcagtaaaa

SEQ ID NO: 286 - DNA, 21 bp, Artificial Sequence
  Synthetic Construct
ctgctgacagaagactgaaag

SEQ ID NO: 287 - DNA, 21 bp, Artificial Sequence
  Synthetic Construct
taagcaagtgagaggcacatt

SEQ ID NO: 288 - DNA, 21 bp, Artificial Sequence
  Synthetic Construct
aaggatggaaaactacattga

SEQ ID NO: 289 - DNA, 21 bp, Artificial Sequence
  Synthetic Construct
tttgaaggatggaaaactaca

SEQ ID NO: 290 - DNA, 21 bp, Artificial Sequence
  Synthetic Construct
ccatccttcaaataaacatgt

SEQ ID NO: 291 - DNA, 21 bp, Artificial Sequence
  Synthetic Construct
catccttcaaataaacatgtc

SEQ ID NO: 292 - DNA, 21 bp, Artificial Sequence
  Synthetic Construct
ttaaaataacttcaatgtagt

SEQ ID NO: 293 - DNA, 21 bp, Artificial Sequence
  Synthetic Construct
aaataacttcaatgtagtttt

SEQ ID NO: 294 - DNA, 21 bp, Artificial Sequence
  Synthetic Construct
taaaataacttcaatgtagtt

SEQ ID NO: 295 - DNA, 21 bp, Artificial Sequence
  Synthetic Construct
aaaataacttcaatgtagttt

SEQ ID NO: 296 - DNA, 21 bp, Artificial Sequence
  Synthetic Construct
aaaaaatatataagttttact

SEQ ID NO: 297 - DNA, 21 bp, Artificial Sequence
  Synthetic Construct
ttctccagcaggcagtgggga

SEQ ID NO: 298 - DNA, 21 bp, Artificial Sequence
  Synthetic Construct
aaaaatatataagttttactg

SEQ ID NO: 299 - DNA, 21 bp, Artificial Sequence
  Synthetic Construct
tggtgtgaggaagagcgatgc

SEQ ID NO: 300 - DNA, 21 bp, Artificial Sequence
  Synthetic Construct
cttagcccacagttctccatt

SEQ ID NO: 301 - DNA, 21 bp, Artificial Sequence
  Synthetic Construct
tctccagcaggcagtggggac

SEQ ID NO: 302 - DNA, 21 bp, Artificial Sequence
  Synthetic Construct
gagacagggctgttgtctccc

SEQ ID NO: 303 - DNA, 21 bp, Artificial Sequence
  Synthetic Construct
agacagggctgttgtctccct

SEQ ID NO: 304 - DNA, 20 bp, Artificial Sequence
  Synthetic Construct
atgatgcaagaccttgcagg

SEQ ID NO: 305 - DNA, 20 bp, Artificial Sequence
  Synthetic Construct
gcaaggtcttgcatcatggg

SEQ ID NO: 306 - DNA, 23 bp, Artificial Sequence
  Synthetic Construct
gagaaggactcaccctcatgtgg

SEQ ID NO: 307 - DNA, 20 bp, Artificial Sequence
  Synthetic Construct
gagaccccatcatggtgctc

SEQ ID NO: 308 - DNA, 20 bp, Artificial Sequence
  Synthetic Construct
aaagccaacttctccatcgg

SEQ ID NO: 309 - DNA, 23 bp, Artificial Sequence
  Synthetic Construct
gaagttccgtgtacacgatgata

SEQ ID NO: 310 - DNA, 23 bp, Artificial Sequence
  Synthetic Construct
acacagagaaaaatcactcgccc

SEQ ID NO: 311 - DNA, 23 bp, Artificial Sequence
  Synthetic Construct
gatctggactcagcatttggaag